(19) United States
(12) Patent Application Publication
(10) Pub. No.: US 2024/0271172 A1
(43) Pub. Date: Aug. 15, 2024

(54) METHODS FOR PROCESSING NUCLEIC ACID SAMPLES

(71) Applicant: 10x StoneridgeMall Road, Pleasanton, CA (US)

(72) Inventors: Xinying Zheng, San Jose, CA (US); Zahra Kamila Belhocine, Pleasanton, CA (US)

(21) Appl. No.: 18/410,457

(22) Filed: Jan. 11, 2024

Related U.S. Application Data
(63) Continuation of application No. 17/522,741, filed on Nov. 9, 2021, now abandoned.
(60) Provisional application No. 63/111,821, filed on Nov. 10, 2020.

(51) Int. Cl. C12P 19/34; C12N 15/10; C12Q 1/6806

(52) U.S. Cl. CPC C12P 19/34; C12N 15/1065; C12Q 1/6806

(57) ABSTRACT

The present disclosure provides methods, systems, and kits for processing nucleic acid molecules. A method may comprise providing a template nucleic acid fragment (e.g., within a cell, cell bead, or cell nucleus) within a partition (e.g., a droplet or well) and subjecting the template nucleic acid fragment to one or more processes including a barcoding process and a single primer extension or amplification process. The processed template nucleic acid fragment may then be recovered from the partition and subjected to further amplification to provide material for subsequent sequencing analysis. The methods provided herein may permit simultaneous processing and analysis of both DNA and RNA molecules (e.g., gRNA molecules) originating from the same cell, cell bead, or cell nucleus.

### CROSS REFERENCE

This application is a continuation of U.S. patent application Ser. No. 17/522,741, filed Nov. 9, 2021, which claims the benefit of U.S. Provisional Patent Application No. 63/111,821, filed Nov. 10, 2020, each of which is incorporated by reference herein in its entirety.

### BACKGROUND

A sample may be processed for various purposes, such as detection, identification, quantification, and characterization of a type of moiety within the sample. The sample may be a biological sample. Biological samples may be processed, such as for detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Biological samples in partitions may be subjected to various processes, such as chemical processes or physical processes. Samples in partitions may be subjected to heating or cooling, or chemical reactions, such as to yield species that may be qualitatively or quantitatively processed.

A biological sample may comprise one or more nucleic acid molecules, such as one or more deoxyribonucleic acid (DNA) molecules and/or one or more ribonucleic acid (RNA) molecules. Methods of processing such a biological sample may vary depending on the types of nucleic acid molecules included therein.

### SUMMARY

The present disclosure provides methods, systems, and kits for processing multiple different types of nucleic acid molecules in tandem. The methods provided herein may allow for analysis of various deoxyribonucleic acid (DNA) molecules and/or ribonucleic acid (RNA) molecules from the same biological particle (e.g., cell, cell bead, or cellular component such as a cell nucleus). DNA and/or RNA molecules may originate from the biological particle. Alternatively or in addition, DNA and/or RNA molecules may be expressed in the biological particle following perturbation of the biological particle involving, for example, introduction of guide RNA (gRNA) and a Clustered Regularly Interspaced Short Palindromic Repeat (CRISPR)-associated (Cas) protein or their precursors. Analysis of different types of nucleic acid molecules may be performed simultaneously or near simultaneously. The methods provided herein may comprise use of a partitioning scheme in which materials (e.g., different types of target nucleic acid molecules, such as target nucleic acid molecules included within a cell, cell bead, or nucleus) are distributed between a plurality of partitions, such as a plurality of droplets or wells. Materials (e.g., target nucleic acid molecules) may be co-partitioned with one or more reagents to facilitate processing of target nucleic acid molecules, such as one or more enzymes, beads (e.g., beads comprising nucleic acid barcode molecules), primers, oligos, lysing or permeabilizing agents, buffers, or other reagents. The methods provided herein may comprise generating a barcoded nucleic acid product (e.g., within a partition of a plurality of partitions) corresponding to each of various different target nucleic acid molecules (e.g., DNA and RNA molecules), prior to subjecting the barcoded nucleic acid products to one or more amplification processes (e.g., polymerase chain reaction (PCR), which may optionally be performed in bulk).

Target nucleic acid molecules (e.g., DNA and/or RNA molecules) may initially be included in a cell, cell bead, or cell nucleus and/or may be introduced into or expressed by a cell, cell bead, or cell nucleus (e.g., following a perturbation). Accordingly, the methods provided herein provide sample preparation techniques that permit sequencing of nucleic acid molecules from single cells, cell beads, and nuclei of interest.

In eukaryotic genomes, chromosomal DNA winds itself around histone proteins (i.e., “nucleosomes”), thereby forming a complex known as chromatin. The tight or loose packaging of chromatin contributes to the control of gene expression. Tightly packed chromatin (“closed chromatin”) is usually not permissive for gene expression while more loosely packaged, accessible regions of chromatin (“open chromatin”) is associated with the active transcription of gene products. Methods for probing genome-wide DNA accessibility have proven extremely effective in identifying regulatory elements across a variety of cell types and quantifying changes that lead to both activation or repression of gene expression.

One such method is the Assay for Transposase Accessible Chromatin with high-throughput sequencing (ATAC-seq). The ATAC-seq method probes DNA accessibility with an artificial transposon, which inserts specific sequences into accessible regions of chromatin. Because the transposase can only insert sequences into accessible regions of chromatin not bound by transcription factors and/or nucleosomes, sequencing reads can be used to infer regions of increased chromatin accessibility.

Traditional approaches to the ATAC-seq methodology requires large pools of cells, processes cells in bulk, and result in data representative of an entire cell population, but lack information about cell-to-cell variation inherently present in a cell population (see, e.g., Buenrostro, et al., Curr. Protoc. Mol. Biol., 2015 Jan. 5; 21.29.1-21.29.9). While single cell ATAC-seq (scATAC-seq) methods have been developed, they suffer from several limitations. For example, scATAC-seq methods that utilize sample pooling, cell indexing, and cell sorting (see, e.g., Cusanovich, et al., Science, 2015 May 22; 348(6237):910-14) result in high variability and a low number of reads associated with any single cell. Other scATAC-seq methods that utilize a programmable microfluidic device to isolate single cells and perform scATAC-seq in nanoliter reaction chambers (see, e.g., Buenrostro, et al., Nature, 2015 Jul. 23; 523(7561):486-90) are limited by the throughput of the assay and may not generate personal epigenomic profiles on a timescale compatible with clinical decision-making.

In an aspect, provided herein is a method for nucleic acid processing, comprising: (a) providing a plurality of partitions comprising a partition, wherein the partition comprises: (i) a biological particle comprising (1) a deoxyribonucleic acid (DNA) molecule and (2) a vector comprising a guide ribonucleic acid (gRNA) sequence or a gRNA molecule comprising the gRNA sequence; (ii) a plurality of nucleic acid barcode molecules, wherein the plurality of nucleic acid barcode molecules comprises a first nucleic acid barcode molecule and a second nucleic acid barcode molecule, wherein the first nucleic acid barcode molecule and the second nucleic acid barcode molecule comprise a common barcode sequence, and wherein the second nucleic acid barcode molecule comprises an adapter sequence; and (iii) a plurality of primer molecules, wherein the plurality of primer molecules comprises a first primer molecule and a second primer molecule, wherein the first primer molecule comprises a sequence complementary to the adapter sequence; (b) attaching (i) the first primer molecule to the vector or the gRNA molecule, to generate an extension product comprising a sequence complementary to the gRNA sequence and the sequence complementary to the adapter sequence, and (ii) the second primer molecule to the extension product or derivative thereof to generate a second extension product; and (c) using (i) the DNA molecule or a derivative thereof and the first nucleic acid barcode molecule to generate a first barcoded nucleic acid molecule and (ii) the extension product or a derivative thereof and the second nucleic acid barcode molecule to generate a second barcoded nucleic acid molecule, wherein the first barcoded nucleic acid molecule comprises the common barcode sequence, or a complement thereof, and wherein the second barcoded nucleic acid molecule comprises the common barcode sequence, or a complement thereof.

In some embodiments, the biological particle is a cell, cell bead, or cell nucleus. In some embodiments, the cell or the cell nucleus is permeabilized. In some embodiments, the method further comprises lysing or permeabilizing the biological particle within the partition to provide access to the DNA molecule and the vector or the gRNA molecule therein. In some embodiments, the method further comprises processing an open chromatin structure of the biological particle with a transposase to yield the DNA molecule. In some embodiments, the processing is performed prior to (a). In some embodiments, the processing occurs in the partition. In some embodiments, transposase is included in a transposase-nucleic acid complex that comprises (i) a first transposase nucleic acid molecule comprising a first transposon end sequence and a first sequencing primer or portion thereof, or a complement thereof and (ii) a second transposase nucleic acid molecule comprising a second transposon end sequence and a second sequencing primer or portion thereof, or a complement thereof. In some embodiments, the first transposon end sequence and the second transposon end sequence are the same, and wherein the first transposon end sequence and the second transposon end sequence are hybridized to complementary sequences. In some embodiments, the gRNA molecule is a single guide RNA (sgRNA) molecule. In some embodiments, the method further comprises, prior to (a), delivering to the biological particle a molecule comprising (i) a sequence of the vector or the gRNA molecule or (ii) a sequence complementary to a sequence of the vector or the gRNA molecule. In some embodiments, the molecule comprises the vector. In some embodiments, the vector is a plasmid vector or viral vector. In some embodiments, the viral vector is a lentiviral vector. In some embodiments, the vector comprises a barcode sequence that identifies the gRNA molecule. In some embodiments, the vector or the gRNA molecule comprises a barcode sequence that identifies the gRNA sequence. In some embodiments, the plurality of nucleic acid barcode molecules is coupled to a particle. In some embodiments, the particle is a bead. In some embodiments, the particle is a gel bead. In some embodiments, the particle is dissolvable or degradable. In some embodiments, the method further comprises subjecting the partition to conditions sufficient to at least partially dissolve or degrade the particle. In some embodiments, the plurality of nucleic acid barcode molecules is releasably coupled to the particle. In some embodiments, at least a subset of the plurality of nucleic acid barcode molecules is releasable from the particle upon application of a stimulus. In some embodiments, the stimulus is a chemical stimulus. In some embodiments, the chemical stimulus is a reducing agent. In some embodiments, the method further comprises subjecting the partition to conditions sufficient to release at least a subset of the plurality of nucleic acid barcode molecules from the particle. In some embodiments, the plurality of nucleic acid barcode molecules is coupled to the particle via a plurality of labile moieties. In some embodiments, the first nucleic acid barcode molecule and the second nucleic acid barcode molecule comprise the same nucleic acid sequence. In some embodiments, generating the first barcoded nucleic acid molecule comprises performing a ligation reaction, a primer extension reaction, a nucleic acid amplification reaction, or a combination thereof. In some embodiments, generating the first barcoded nucleic acid molecule comprises performing a ligation reaction. In some embodiments, generating the first barcoded nucleic acid molecule comprises performing a primer extension reaction. In some embodiments, the first nucleic acid barcode molecule comprises a flow cell sequence, or a complement thereof; a sequencing primer, or portion thereof; a unique molecular identifier sequence; or a combination thereof. In some embodiments, the DNA molecule comprises one or more gaps, and further comprising, prior to (c), subjecting the DNA molecule to an extension process to fill the one or more gaps. In some embodiments, the first primer molecule comprises a first primer sequence configured to hybridize to a first sequence of the vector or the gRNA molecule and the second primer molecule comprises a second primer sequence configured to hybridize to a second sequence of the vector or the gRNA molecule that is different from the first sequence. In some embodiments, the first sequence of the vector or the gRNA molecule and the second sequence of the vector or the gRNA molecule are disposed at or near opposite ends of the gRNA molecule. In some embodiments, (b) comprises subjecting the partition to conditions sufficient for the first primer sequence of the first primer molecule to hybridize to the first sequence of the vector or the gRNA molecule and for the second primer sequence of the second primer molecule to hybridize to the second sequence of the vector or the gRNA molecule. In some embodiments, the first primer molecule comprises a first primer sequence configured to hybridize to a first sequence of the vector or the gRNA molecule that is disposed upstream of the barcode sequence, and wherein the second primer molecule comprises a second primer sequence configured to hybridize to a second sequence of the vector or the gRNA molecule that is disposed downstream from the first sequence and the barcode sequence. In some embodiments, the first sequence of the vector or the gRNA molecule and the second sequence of the vector or the gRNA molecule are disposed at or near opposite ends of the vector or the gRNA molecule. In some embodiments, (b) comprises subjecting the partition to conditions sufficient for the first primer sequence of the first primer molecule to hybridize to the first sequence of the vector or the gRNA molecule and for the second primer sequence of the second primer molecule to hybridize to the second sequence of the vector or the gRNA molecule. In some embodiments, generating the second barcoded nucleic acid molecule comprises subjecting the partition to conditions sufficient for the sequence complementary to the adapter sequence to hybridize the adapter sequence of the second nucleic acid barcode molecule. In some embodiments, the second nucleic acid barcode molecule comprises a unique molecular identifier sequence, a sequencing primer or portion thereof, or a combination thereof. In some embodiments, the method further comprises (d) recovering the first barcoded nucleic acid molecule or a derivative thereof and the second barcoded nucleic acid molecule or a derivative thereof from the partition. In some embodiments, the method further comprises, subsequent to (d), using (i) the first barcoded nucleic acid molecule or the derivative thereof to generate a first plurality of amplification products and (ii) the second barcoded nucleic acid molecule or the derivative thereof to generate a second plurality of amplification products. In some embodiments, generating the first plurality of amplification products comprises performing one or more nucleic acid amplification reactions using the first barcoded nucleic acid molecule or the derivative thereof. In some embodiments, generating the second plurality of amplification products comprises performing one or more nucleic acid amplification reactions using the second barcoded nucleic acid molecule or the derivative thereof. In some embodiments, performing the one or more nucleic acid amplification reactions comprises attaching one or more flow cell sequences to the first barcoded nucleic acid molecule or the derivative thereof or the second barcoded nucleic acid molecule or the derivative thereof. In some embodiments, the plurality of primer molecules is provided in a solution. In some embodiments, the plurality of primer molecules is coupled to a particle. In some embodiments, the partition is a droplet among a plurality of droplets. In some embodiments, the method further comprises, (d) recovering the first barcoded nucleic acid molecule or a derivative thereof and the second barcoded nucleic acid molecule or a derivative thereof from the partition, wherein (d) comprises breaking or disrupting the droplet. In some embodiments, the partition is a well. In some embodiments, the method further comprises repeating (a)-(c) for additional partitions among the plurality of partitions. In some embodiments, the first primer molecule and/or the second primer molecule comprises one or more functional sequences. In some embodiments, the one or more functional sequences comprise a sequencing primer sequence, a sequencing primer binding sequence, a partial sequencing primer sequence, or a partial sequencing primer binding sequence. In some embodiments, (c) is performed within the partition. In some embodiments, the extension product is generated using the vector as template. In some embodiments, the extension product is generated using an mRNA as template, wherein the mRNA is transcribed from the vector. In some embodiments, the extension product comprises the barcode sequence that identifies the gRNA molecule.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7A shows a cross-section view of another example of a microfluidic channel structure with a geometric feature for controlled partitioning. FIG. 7B shows a perspective view of the channel structure of FIG. 7A.

FIG. 8 illustrates an example of a barcode carrying bead.

FIG. 9 illustrates a transposase-nucleic acid complex comprising a transposase, a first double-stranded oligonucleotide comprising a transposon end sequence and a first primer sequence and a second double-stranded oligonucleotide comprising a transposon end sequence and a second primer sequence.

FIG. 10 illustrates a transposase-nucleic acid complex comprising a transposase, a first double-stranded oligonucleotide comprising a transposon end sequence and first and second primer sequences and a second double-stranded oligonucleotide comprising a transposon end sequence and third and fourth primer sequences.

FIG. 11 illustrates a transposase-nucleic acid complex comprising a transposase, a first hairpin molecule, and a second hairpin molecule.

FIGS. 12A-12C illustrate schemes for tandem ATAC and RNA processing.

FIG. 13 illustrates a schematic workflow for tandem ATAC and RNA processing.

FIG. 14 illustrates a scheme for tandem ATAC and RNA processing.

FIG. 15 illustrates a scheme for tandem ATAC and RNA processing.

FIG. 16 illustrates a scheme for tandem ATAC and RNA processing.

FIG. 17 illustrates a scheme for T7 mediated linear amplification.

FIG. 18 illustrates a scheme for tandem ATAC and RNA processing.

FIGS. 19A and 19B show beads for use according to the methods of the present disclosure.

FIG. 20 shows a computer system that is programmed or otherwise configured to implement methods provided herein.

FIG. 21 schematically illustrates example labelling agents with nucleic acid molecules attached thereto.

FIG. 22A schematically shows an example of labelling agents. FIG. 22B schematically shows another example workflow for processing nucleic acid molecules. FIG. 22C schematically shows another example workflow for processing nucleic acid molecules.

FIG. 23 schematically shows another example of a barcode-carrying bead.

FIG. 24 illustrates another example of a barcode carrying bead.

FIG. 25 schematically illustrates an example workflow for processing nucleic acid molecules.

FIG. 26 schematically illustrates an example microwell array.

The instant application contained at least one drawing executed in color. Copies of this patent application publication with color drawing(s) will be provided by the Office upon request and payment of the necessary fee.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The present disclosure provides methods, systems, and kits for processing multiple types of nucleic acid molecules. The methods, systems, and kits provided herein may facilitate sample preparation for sequencing of nucleic acid molecules included in biological particles such as cells, cell beads, or cell nuclei of interest. For example, the present disclosure provides methods for processing both deoxyribonucleic acid (DNA) and ribonucleic acid (RNA) molecules included within a biological particle (e.g., cell, cell bead, or cell nucleus of interest). DNA molecules processed according to the methods, systems, and kits provided herein may reflect perturbations introduced to the biological particle using, for example, a Clustered Regularly Interspaced Short Palindromic Repeats (CRISPR)-associated (Cas) protein, such as a CRISPR-Cas system coupled to or configured to couple to a guide RNA (gRNA) molecule. RNA molecules may be gRNA molecules directly introduced into the biological particle or expressed within the biological particle. Partitioning and barcoding schemes may be utilized to facilitate identification of resultant sequencing reads with the biological particle from which they are derived.

The methods may comprise performing Assay for Transposase Accessible Chromatin with high-throughput sequencing (ATAC-seq) and RNA sequencing (RNA-seq) assays in tandem. The methods, systems, and kits provided herein may combine ATAC-seq and perturbation sequencing (Perturb-seq), or aspects thereof. Such methods may generally be referred to herein as Perturbation-indexed Assay for Transposase Accessible Chromatin using Sequencing (Perturb-ATAC-seq or Perturb-ATAC). Accordingly, the methods, systems, and kits provided herein may facilitate high-throughput, simultaneous measurement of CRISPR-perturbations and chromatin state in single biological particles (e.g., single cells). Single biological particles may be perturbed by introduction of, for example, gRNA molecules and then profiled for simultaneous detection of gRNA signatures and open chromatin sites. The methods, systems, and kits provided herein may reveal regulatory factors that control cis-element accessibility and/or trans-factor occupancy. In some cases, the methods, systems, and kits provided herein may reveal nucleosome positioning and/or regulatory nodules of coordinated activity in a cell type, such as coordinated trans-factor activities, synergistic activities of co-binding transcription factors (TFs) on cis-elements, etc. The methods provided herein may be performed in a high throughput manner to facilitate obtaining single biological particle (e.g., single cell) data, including epigenomic variability.

### Perturb-ATAC-Seq

In an aspect, the present disclosure provides methods, systems, and kits for processing multiple types of nucleic acid molecules in individual biological particles (e.g., cells, cell beads, or cell nuclei). The methods, systems, and kits provided herein may facilitate parallel analysis of perturbations introduced to biological particles. For example, DNA (e.g., genomic DNA) and perturbation agents (e.g., guide RNA molecules or a precursor, portion, or a derivative thereof, any of which may be associated with the gRNA or a gRNA-identifying barcode, or optionally, other components of a CRISPR-Cas system) introduced to a biological particle (e.g., cell, cell bead, or cell nucleus) may be analyzed using parallel ATAC-seq and other methods (e.g., as described herein). Accordingly, the present disclosure provides a method for nucleic acid processing comprising providing a partition (e.g., among a plurality of partitions, such as a droplet among a plurality of droplets or a well among a plurality of wells) comprising a biological particle (e.g., cell, cell bead, or cell nucleus) comprising a DNA molecule and a gRNA molecule. The biological particle may be lysed, permeabilized, fixed, cross-linked, or otherwise manipulated to provide access to one or more nucleic acid molecules (e.g., template or target nucleic acid molecules, such as a DNA molecule and a gRNA molecule) therein. The partition may also comprise a first nucleic acid barcode molecule and a second nucleic acid barcode molecule that may have the same or different sequences and may comprise a common barcode sequence. The first and second nucleic acid barcode molecules may each be coupled to a particle, such as a gel bead. Within the partition, the DNA molecule, or a derivative thereof, and the first nucleic acid barcode molecule may be used to generate a first barcoded nucleic acid molecule. The gRNA molecule, or a derivative thereof, and the second nucleic acid barcode molecule may be used to generate a second barcoded nucleic acid molecule. The first barcoded nucleic acid molecule may comprise the common barcode sequence, or a complement thereof. The second barcoded nucleic acid molecule may also comprise the common barcode sequence, or a complement thereof. The first barcoded nucleic acid molecule, or a derivative thereof, and the second barcoded nucleic acid molecule, or a derivative thereof, may be recovered from the partition. The recovered products may be subjected to additional downstream analysis including, for example, nucleic acid sequencing.

The present disclosure also provides a method for nucleic acid processing comprising providing a partition (e.g., among a plurality of partitions, such as a droplet among a plurality of droplets or a well among a plurality of wells) comprising a biological particle (e.g., cell, cell bead, or cell nucleus) comprising a DNA molecule and a gRNA molecule. The biological particle may be lysed, permeabilized, fixed, cross-linked, or otherwise manipulated to provide access to one or more nucleic acid molecules (e.g., template or target nucleic acid molecules, such as a DNA molecule and/or a gRNA molecule) therein. The partition may also comprise a plurality of nucleic acid barcode molecules, which plurality of nucleic acid barcode molecules comprises a first nucleic acid barcode molecule and a second nucleic acid barcode molecule. The first nucleic acid barcode molecule and the second nucleic acid barcode molecule may have the same or different sequences and may comprise a common barcode sequence. The second nucleic acid barcode molecule may comprise an adapter sequence. The partition may also comprise a plurality of primer molecules, which plurality of primer molecules may comprise a first primer molecule and a second primer molecule. The first primer molecule may comprise a sequence complementary to the adapter sequence, or a complement thereof. Within the partition (e.g., droplet or well), the DNA molecule, or a derivative thereof, and the first nucleic acid barcode molecule may be used to generate a first barcoded nucleic acid molecule (e.g., via one or more ligation, primer extension, or nucleic acid amplification reactions, or a combination thereof). The gRNA molecule (or a precursor, portion, or a derivative thereof, any of which may be associated with the gRNA or a gRNA-identifying barcode); the second nucleic acid barcode molecule; the first primer molecule; and the second primer molecule may be used to generate a second barcoded nucleic acid molecule (e.g., via one or more ligation, primer extension, or nucleic acid amplification reactions, or a combination thereof). The first barcoded nucleic acid molecule may comprise the common barcode sequence, or a complement thereof. The second barcoded nucleic acid molecule may also comprise the common barcode sequence, or a complement thereof. The first barcoded nucleic acid molecule, or a derivative thereof, and the second barcoded nucleic acid molecule, or a derivative thereof, may be recovered from the partition (e.g., released from the partition, e.g., pooled with contents of other partitions of a plurality of partitions, such as by disrupting a droplet in which they are contained). The first barcoded nucleic acid molecule, or a derivative thereof, and the second barcoded nucleic acid molecule, or a derivative thereof, may undergo one or more additional processing steps in bulk. For example, the first barcoded nucleic acid molecule, or a derivative thereof, and the second barcoded nucleic acid molecule, or a derivative thereof may undergo a gap filling process, a dA tailing process, a terminal-transferase process, a phosphorylation process, a ligation process, a nucleic acid amplification process, or a combination thereof. For example, the first barcoded nucleic acid molecule, or a derivative thereof, and the second barcoded nucleic acid molecule, or a derivative thereof, may be subjected to conditions sufficient to undergo one or more polymerase chain reactions (PCR, such as sequence independent PCR) to generate amplification products corresponding to the first barcoded nucleic acid molecule, or a derivative thereof, and the second barcoded nucleic acid molecule, or a derivative thereof. Sequences of such amplification products can be detected using, for example, a nucleic acid sequencing assay and used to identify sequences of the DNA molecule and/or the gRNA molecule of the biological particle (e.g., cell, cell bead, or cell nucleus) from which they derive.

A biological sample (e.g., a nucleic acid sample) may comprise one or more biological particles, which one or more biological particles may be, for example, cells, cell beads, and/or cell nuclei. A biological sample may also comprise tissue, which tissue may comprise one or more cells, cell beads, and/or cell nuclei. In some cases, a biological sample may comprise a plurality of cells comprising a plurality of cell nuclei. In some cases, a biological sample may comprise a plurality of cell nuclei, which plurality of cell nuclei are not included within cells (e.g., other components of the cell have degraded, dissociated, dissolved, or otherwise been removed). A biological sample may comprise a plurality of cell-free nucleic acid molecules (e.g., nucleic acid molecules that are not included within cells). For example, a biological sample may comprise a plurality of cell-free fetal DNA (cffDNA) or circulating tumor DNA (ctDNA) or other cell-free nucleic acid molecules (e.g., deriving from degraded cells). Such a biological sample may be processed to separate such cell-free nucleic acid molecules from cells, cell beads, and/or cell nuclei, which cells, cell beads, and/or cell nuclei may be subjected to further processing (e.g., as described herein).

A biological particle (e.g., cell, cell bead, or cell nucleus) may be of any useful type. For example, a cell may be an immune cell, such as a B cell or T cell. A biological particle may be derived from any useful source. A biological particle may be isolated and/or captured from a source. In an example, a plurality of cells of a sample may be subjected to a sorting process, and a subset of the plurality of cells may be selected for further analysis or processing. Sorting may comprise, for example, fluorescence-based (e.g., flow cytometry) and/or magnetic sorting. Selection of biological particles for further analysis may comprise isolation and/or capture protocol based on surface-available proteins and/or antibodies.

Biological particles (e.g., cells, cell beads, or cell nuclei) may be pre-sorted based on a transduced marker prior to analysis (e g., as described herein) by fluorescence-activated cell sorting (FACS) or magnetic-activated cell sorting (MACS) For example, cells may be transduced with a fluorescent protein (e.g., GFP, YFP, CFP, mCherry, mRuby, etc.). In some cases, the fluorescent protein may be transduced with a sgRNA cassette. Selection for cells expressing the sgRNA cassette may be done by a pre-sorting mechanism. In some cases, one or more selection markers may be used. For example, cells may be transduced with a drug-resistance (e g., puromycin, blasticidin resistance) gene to select for the sgRNA vector.

A plurality of biological particles (e.g., cells, cell beads, or cell nuclei) may comprise any number of biological particles, e.g., about 500 to about 105 or more biological particles, about 500 to about 100,000 biological particles, about 500 to about 50,000 biological particles, about 500 to about 10,000 biological particles, about 50 to 1000 biological particles, about 1 to 500 biological particles, about 1 to 100 biological particles, about 1 to 50 biological particles, or a single cell. In some cases, the plurality of biological particles can consist of less than about 1000, about 2000, about 3000, about 4000, about 5000, about 6000, about 7000, about 8000, about 9000, about 10,000, about 15,000, about 20,000, about 25,000, about 30,000, about 40,000, about 50,000, about 60,000, about 70,000, about 80,000, about 90,000, about 100,000, about 120,000, about 140,000, about 160,000, about 180,000, about 200,000, about 250,000, about 300,000, about 350,000, about 400,000, about 450,000, about 500,000, about 600,000, about 700,000, about 800,000, about 900,000, or about 1,000,000 biological particles. In other cases, the plurality of biological particles can comprise of more than about 1000, about 2000, about 3000, about 4000, about 5000, about 6000, about 7000, about 8000, about 9000, about 10,000, about 15.000, about 20,000, about 25,000, about 30,000, about 40,000, about 50,000, about 60,000, about 70,000, about 80,000, about 90,000, about 100,000, about 120,000, about 140,000, about 160,000, about 180,000, about 200,000, about 250,000, about 300,000, about 350,000, about 400,000, about 450,000, about 500,000, about 600,000, about 700,000, about 800,000, about 900,000, or about 1,000,000 biological particles.

Nucleic acid molecules included within a biological particle may include, for example, DNA molecules and RNA molecules. For example, a biological sample may comprise genomic DNA comprising chromatin (e.g., within a cell, cell bead, or cell nucleus). A biological sample may comprise a plurality of RNA molecules, which plurality of RNA molecules may be of one or more different types. For example, the plurality of RNA molecules may comprise gRNA molecules, such as gRNA molecules introduced to the biological particle (e.g., as described herein) and/or expressed by the biological particle. RNA molecules such as mRNA molecules may comprise a polyA sequence. At least a subset of a plurality of RNA molecules included in a cell or cell bead may be present in a cell nucleus.

A nucleic acid molecule may be a guide RNA (gRNA) molecule or a precursor, portion, or a derivative thereof, any of which can be associated with the gRNA or a gRNA-identifying barcode. Accordingly, the nucleic acid molecule may comprise a DNA or RNA molecule. In some aspects, the nucleic acid molecule may be a vector comprising a sequence of a guide ribonucleic acid (gRNA) molecule and/or a barcode sequence that identifies the gRNA. In some examples, the nucleic acid molecule (e.g., mRNA molecule) may be a nucleic acid transcript comprising the sequence of the gRNA and/or a barcode sequence that identifies the gRNA molecule. A guide RNA molecule may be a single guide RNA (sgRNA) molecule. A gRNA molecule may comprise a sgRNA spacer sequence. A gRNA molecule may be useful in nucleic acid molecule editing (e.g., site specific nucleic acid molecule editing) involving a CRISPR-CRISPR-associated (Cas) protein (e.g., CRISPR-Cas) system. A gRNA molecule may comprise any useful number of nucleotides, including any nucleotide analogs (e.g., as described herein), arranged in any useful order. A gRNA molecule may comprise one or more sequences, which one or more sequences may be arranged in any useful order. For example, a gRNA molecule may comprise a CRISPR RNA (crRNA) sequence configured to interact with a specific region of a genome (e.g., with a specific gene). A crRNA sequence may be considered to be a variable sequence as it may vary based on a genome of interest rather than based on a CRISPR-Cas system with which the gRNA molecule may be configured to associate. A gRNA molecule may also comprise a trans-activating CRISPR RNA (tracrRNA) sequence. A tracrRNA sequence may be configured to interact with a Cas protein of a CRISPR-Cas system. A tracrRNA sequence may be considered to be a constant sequence as it may vary based on the CRISPR-Cas system with which the gRNA molecule may be configured to associate rather than a genome of interest. A gRNA molecule comprising both crRNA and tracrRNA sequences in a single molecule may be referred to as a single guide RNA (sgRNA) molecule. In some cases, tracrRNA and crRNA sequences may be included in separate molecules that may together make up a gRNA molecule or system. A gRNA molecule may comprise one or more additional sequences. For example, a gRNA molecule may comprise one or more spacer sequences and/or one or more sequences configured to confer a secondary structure to the gRNA molecule. In an example, a gRNA molecule (e.g., sgRNA molecule) may comprise one or more sequences configured to form one or more loop (e.g., stem loop and/or tetraloop) or hairpin structures, which one or more sequences may be separated by one or more spacer sequences. A gRNA molecule may include 1, 2, 3, 4, or more loop or hairpin structures. For example, a gRNA molecule configured to interact with a Cas9 protein in a CRISPR-Cas system may comprise a first structure comprising a tetraloop as well as three stem loop structures. One or more structures of a gRNA molecule may facilitate interaction (e.g., attachment) between the gRNA molecule and a Cas protein of a CRISPR-Cas system. A gRNA molecule may also comprise one or more sequences that may encode identifying information, such as one or more barcode sequences. A gRNA molecule may also comprise a homopolymeric sequence such as a polyA or polyT sequence. Such a sequence may be related to transcriptional processing of RNA molecules, e.g., within a biological particle.

A tracrRNA sequence of a gRNA molecule may be referred to as a “scaffold” sequence and may be configured to interact with a Cas protein of a CRISPR-Cas system. A tracrRNA sequence may have any useful length and base composition, and any other useful characteristics. In some examples, a tracrRNA sequence may comprise one or more regions that are configured to associate with one another to provide secondary structures including loops (e.g., stem loops) and/or hairpins. A tracrRNA sequence may be specific to a given type of Cas protein. A Cas protein may be a nuclease, such as a type II nuclease (e.g., wild type or modified versions thereof). Examples of Cas proteins include, but are not limited to, Cas1, Cas2, Cas3, Cas4, Cas5, Cas6, Cas7, Cas8, Cas9, Cas10, Cas11, Cas12, Cas13, and variants thereof. Cas9 may be derived from Staphylococcus pyogenes or S. thermophilus and may comprise about 1368 amino acids. Additional examples of Cas proteins include, but are not limited to, CasX (e.g., derived from Plantomycetes and Deltaproteobacteria, made up of about 980 amino acids), CasY (e.g., derived from Candidate Phyla Radiation, made up of about 1200 amino acids), Csn2, C2c1, C2c3, C2c2 (e.g., Cas13a), and Cpf1 (e.g., alternately referred to as Cas12a, derived from Prevotella and Francisella, made up of about 1200-1300 amino acids) proteins. In some cases, a Cas protein may be catalytically inactive such that a CRISPR-Cas system including the catalytically inactive Cas protein may be used to anchor the CRISPR-Cas system to a target nucleic acid sequence without cleaving one or more strands of a nucleic acid molecule including the target nucleic acid sequence. An example of a catalytically inactive Cas protein is “dead” Cas9 (e.g., dCas9), which may be generated by introducing silencing mutations of the RuvC1 and HNH nuclease domains (D10A and H841/A) of a Cas9 protein. Another example of a catalytically inactive Cas protein is “dead” Cpf1 (e.g., dCpf1), which may similarly be generated by introducing a mutation in a RuvC domain of a Cpf1 protein. A catalytically inactive Cas protein of a CRISPR-Cas system may be coupled to a separate endonuclease to effect controlled nucleic acid molecule editing.

A CRISPR-Cas system may comprise an RNA-guided nuclease (e.g., endonuclease), which nuclease (e.g., Cas protein) may be guided by a gRNA molecule. Thus, a CRISPR-Cas system is a protein-RNA complex that uses a gRNA molecule as a guide to localize the complex to a target DNA sequence via base-pairing. Various Cas proteins can be used. Such proteins are often classified into multiple different classes, (e.g., classes 1 and 2), types (e.g., types I, II, III, and IV), and subtypes based on, e.g., their respective signature genes and typical organization of respective loci. For example, class 1 CRISPR-Cas systems include effectors having multiple subunits, while class 2 CRISPR-Cas systems include effectors that may be single large proteins. Class 1 CRISPR-Cas systems may be of type I, III, or IV and various subtypes and may not require a tracrRNA sequence, while Class 2 CRISPR-Cas systems may be of type II, V, or VI and may require a tracrRNA sequence. Type II and V systems are configured to target DNA and generally require tracrRNA to function. Type II systems are often characterized by the use of Cas9, while type V systems often use Cas12 endonucleases such as Cpf1 (e.g., Cas12a).

A crRNA sequence of a gRNA molecule may be configured to interact with a specific region of a genome. For example, a crRNA sequence may be configured to interact with a specific gene for the purposes of deleting or modifying a sequence of the gene, such as by introducing a new nucleotide or nucleic acid sequence. A crRNA sequence may be configured to locate a sequence of interest in a genome to facilitate modification of the genome using the endonuclease activity of a CRISPR-Cas system. Multiple different crRNA sequences may be configured to interact with a same gene (e.g., at different locations within the gene). Binding of a crRNA sequence to genomic DNA may activate the CRISPR-Cas system, thereby permitting genomic editing, which may involve cleavage of one or more strands of a target nucleic acid molecule (e.g., genomic DNA molecule or fragment thereof, such as a tagmented fragment). A crRNA sequence or portion thereof that is complementary to a target sequence may be referred to as a “spacer.” A target sequence may be about 20 bases long. A target sequence (alternately referred to as a “protospacer”) may be disposed near or adjacent to a protospacer adjacent motif (PAM), which may be a 2-6 base sequence that varies based on the Cas protein of a CRISPR-Cas system and is recognizable by the Cas protein. Recognition of a PAM sequence by a Cas protein may destabilize an adjacent or nearby sequence, facilitating interaction between a crRNA sequence, or portion thereof, and thereby pairing RNA and DNA when sequences are sufficiently complementary (e.g., for a DNA-editing system, such as a Class 2, type II CRISPR-Cas system). A common PAM sequence for the S. pyogenes-derived Cas9 protein is 5′-NGG-3′, where N is any nucleobase. A PAM sequence may be a short, nonspecific sequence that may occur frequently at many places throughout a genome.

A gRNA molecule may comprise one or more synthetic sequences and may not be endogenous to a biological particle (e.g., cell, cell bead, or cell nucleus). A gRNA molecule may be exogenous to a biological particle. A gRNA molecule, or a precursor thereof, may be introduced to a biological particle by, for example, electroporation, nucleofection, microinjection, lipofection (e.g., lipid-based transfection, such as liposomal delivery), transfection, transduction, or any other useful method. A gRNA molecule, or a precursor thereof, may be introduced to a biological particle using a viral vector system, such as a retroviral (e.g., lentiviral) vector system. A viral vector system may include a cassette encoding various components including one or more promoters (e.g., a type III RNA polymerase III promoter, such as a U6 promoter, elongation factor promoters, such as EF-1a), proteins (e.g., fluorescent protein or other protein) or protein-encoding sequences, selection agents (e.g., selection proteins, antibiotic (e.g., Puromycin) resistance encoding genes or sequences), gRNA sequences, barcode sequences (e.g., guide barcode sequence), primer sequences or partial primer sequences, or complements thereof, or combinations thereof. For example, a viral vector system may encode an antisense sequence of a gRNA molecule (e.g., sgRNA molecule). Examples of viral vectors can be found in, for example, Dixit et al. “Perturb-Seq: Dissecting Molecular Circuits with Scalable Single-Cell RNA Profiling of Pooled Genetic Screens,” Cell, 2016, 167:1853-1866; Adamson et al. “A multiplexed single-cell CRISPR screening platform enables systematic dissection of the unfolded protein response,” Cell, 2016, 167: 1867-1882; and Datlinger et al. “Pooled CRISPR screening with single-cell transcriptome read-out,” Nature Methods, 2017, 14(3):297-301, each of which is incorporated by reference herein.

A vehicle for providing a gRNA molecule or precursor thereof to a biological particle (e.g., a viral vector) may comprise a barcode (e.g., a gRNA-identifying barcode or guide barcode), which barcode may correspond to the identity of a gRNA. For example, a gRNA-identifying barcode may correspond to the identify of a gRNA molecule encoded by a vector (e.g., a viral vector). Such a barcode may be used for identification of a gRNA molecule or derivative thereof in a given biological particle among a population of biological particles. A gRNA-identifying barcode may have any useful features. Such a barcode may have a length of between 6-20 nucleotides. In some cases, a gRNA-identifying barcode may have a length of at least 10 nucleotides, such as at least 10, 12, 14, 16, 18, 20, 22, 24, 26, 28, 30, or more nucleotides. A barcode may be positioned adjacent to a gRNA-encoding sequence. Each different gRNA molecule or precursor thereof may be associated with a different barcode of a plurality of gRNA-identifying barcodes such that each different crRNA sequence of the different gRNA molecules or precursors thereof may be associated with a different barcode sequence. In some instances, the gRNA molecules or precursors thereof provided to a plurality of biological particles may comprise a target sequence (e.g., crRNA sequence) that is configured to target a sequence associated with a transcription factor, a chromatin modifier, noncoding RNA, or other complex. In some instances, the gRNA molecules or precursors thereof provided to a plurality of biological particles may comprise the same target sequence (e.g., crRNA sequence) In other instances, the gRNA molecules or precursors thereof provided to a plurality of biological particles may comprise different target sequences. For example, a first gRNA molecule or precursor thereof may comprise a first target sequence, and a second gRNA molecule or precursor thereof may comprise a second target sequence.

A precursor of a gRNA molecule (e.g., sgRNA molecule) may be introduced to a biological particle (e.g., as described herein). For example, a precursor of a gRNA molecule may be introduced to a biological particle using a vector delivery system, such as a viral vector delivery system. The precursor may comprise a nucleic acid sequence that is complementary to the sequence of the gRNA molecule (e.g., an antisense sequence). Transcription of the nucleic acid sequence within the biological particle (e.g., using an RNA polymerase III via a U6 promoter) may provide the gRNA molecule in the biological particle. The gRNA molecule may subsequently be used in a gene editing processes using a CRISPR-Cas system and/or analyzed according to the methods provided herein.

Multiple different gRNA molecules or precursors thereof may be introduced to a given biological particle (e.g., at the same or different times, using the same or different methods). For example, a first gRNA molecule or precursor thereof may be introduced to a biological particle (e.g., cell, cell bead, or cell nucleus) at a first time using a first method and a second gRNA molecule or precursor thereof may be introduced to the biological particle at a second time using a second method, where the first time and second time are different. The first and second methods may be the same or different. The first gRNA molecule and the second gRNA molecule may both be sgRNA molecules. The first gRNA molecule and the second gRNA molecule may be configured to interact with the same Cas protein, such as the same Cas9 protein. Accordingly, the first and second gRNA molecules may include one or more common sequences. For example, the first and second gRNA molecules may comprise identical tracrRNA sequences. Alternatively, the first and second gRNA molecules may comprise different tracrRNA sequences. The first and second gRNA molecules may be configured to interact with the same or different genes. For example, the first gRNA molecule may be configured to interact with a first region of genomic DNA encoding a gene and the second gRNA molecule may be configured to interact with a second region of genomic DNA encoding the gene, where the first and second regions are different. The first and second regions may be overlapping or non-overlapping regions. In another example, the first gRNA molecule may be configured to interact with a region of genomic DNA encoding a first gene and the second gRNA molecule may be configured to interact with a region of genomic DNA encoding a second gene, where the first and second genes are different. The first and second genes may relate to similar functions of a biological particle or system (e.g., to similar pathways) or different functions. gRNA molecules configured to interact with different genes or with different regions of the same genes may comprise different crRNA sequences. Accordingly, in an example, a first gRNA molecule comprising a first crRNA sequence and a first tracrRNA sequence may be introduced to a biological particle and a second gRNA molecule comprising a second crRNA sequence and a second tracrRNA sequence may be introduced to the biological particle (e.g., at the same or different times). The first tracrRNA sequence and the second tracrRNA sequence may have the same nucleic acid sequence and be configured to interact with the same Cas protein (e.g., a Cas9 protein). The first crRNA sequence and the second crRNA sequence may have different nucleic acid sequences and be configured to interact with different regions of genomic DNA (e.g., for editing different genes or different regions of the same gene).

Provision of a gRNA molecule or precursor thereof may occur in absence of provision of other components of a CRISPR-Cas system. For example, a gRNA molecule or precursor thereof may be provided to a biological particle that does not include any other components of a CRISPR-Cas system or precursors thereof, such as a biological particle that does not include a Cas protein or precursor thereof. Alternatively, multiple components of a CRISPR-Cas system may be introduced to a biological particle such that the biological particle may be subjected to a genetic editing process.

Multiple components of a CRISPR-Cas system may be delivered to a biological particle at the same or different times. For example, a Cas protein or precursor thereof, such as an mRNA sequence encoding a Cas protein, may be introduced (e.g., delivered) to a biological particle at a first time and a gRNA molecule or precursor thereof may be introduced to the biological particle at a second time that is different than the first time. For example, a cell line may be stably transfected with a Cas protein or precursor thereof such that DNA encoding the Cas protein will be inserted into the cell's genome. Genes for the Cas protein will therefore be passed down to future generations of cells, e.g., following cell division. Expression of a Cas protein may be inducible (e.g., induced by a trigger, such as a drug) or may be constitutive (e.g., occurring at all times). A biological particle that already comprises a Cas protein or genetic material encoding for the Cas protein may separately be provided a gRNA molecule or precursor thereof, such as via a lentiviral vector delivery system. Alternatively, a single delivery system may be used to provide both a Cas protein or precursor thereof and a gRNA molecule or precursor thereof to a biological particle (e.g., cell, cell bead, or cell nucleus).

Provision of a gRNA molecule or precursor thereof and, optionally, other components of a CRISPR-Cas system (e.g., a Cas protein or precursor thereof) may be performed for isolated biological particles and/or populations of biological particles, such as biological particles suspended in media or otherwise disposed in a controlled environment. Provision of a gRNA molecule or precursor thereof may be performed outside of a partition (e.g., droplet or well). Alternatively, provision of a gRNA molecule or precursor thereof may be performed inside a partition (e.g., droplet or well). For example, a plurality of gRNA molecules or precursors thereof may be provided to a population of biological particles (e.g., cells, cell beads, or cell nuclei) in a bulk solution. In another example, a plurality of gRNA molecules or precursors thereof may be provided to a population of biological particles (e.g., cells, cell beads, or cell nuclei) partitioned amongst a plurality of partitions, such as a plurality of wells. gRNA molecules or precursors thereof may be provided to a population of biological particles in a spatially resolved manner. gRNA molecules or precursors thereof (and, optionally, other components of a CRISPR-Cas system) may be provided to a population of biological particles (e.g., cells, cell beads, or cell nuclei) in a two-dimensional array and/or a three-dimensional array, such as to a population of biological particles as components of a tissue section or sample, such as a tissue section on a substrate. For example, a first gRNA molecule or precursor thereof may be provided to a first biological particle of a population of biological particles (e.g., cells, cell beads, or cell nuclei) at a first spatial location of a plurality of spatial locations and a second gRNA molecule or precursor thereof may be provided to a second biological particle of the population of biological particles at a second spatial location of the plurality of spatial locations. This process may be repeated for a plurality of gRNA molecules or precursors thereof. Providing a gRNA molecule or precursor thereof to a spatial location may comprise spotting or otherwise distributing the gRNA molecule or precursor thereof to the spatial location. The gRNA molecule or precursor thereof, or a vehicle used to provide the same to a spatial location, may comprise a label such as a nucleic acid barcode sequence, a fluorescent label, or any other label. gRNA molecules or precursors thereof, or vehicles used to provide the same to various spatial locations, may be provided to a plurality of biological particles according to a pre-defined spatial configuration or pattern. Alternatively, gRNA molecules or precursors thereof, or vehicles used to provide the same to various spatial locations, may be randomly provided to a plurality of biological particles. A pattern for distributing gRNA molecules or precursors thereof to a plurality of biological particles may be based upon pre-defined spatial configurations, such as a grid-like or radial pattern applied to the plurality of biological particles, and/or may reflect aspects of sample differentiation, such as different sample preparation methods or cell lines. For example, a first set of biological particles of a first origin (e.g., of a first cell line and/or sample) may be disposed at a first spatial location or plurality of spatial locations (such as a plurality of spatial locations of a well plate) and a second set of biological particles of a second origin (e.g., of a second cell line and/or sample) may be disposed at a second spatial location or plurality of spatial locations (such as a plurality of spatial locations of a well plate), which second spatial location or plurality of spatial locations may be different than the first spatial location or plurality of spatial locations. A first set of gRNA molecules or precursors thereof may be provided to the first set of biological particles and a second set of gRNA molecules or precursors thereof may be provided to the second set of biological particles.

In one non-limiting example, a gRNA molecule, or a precursor thereof, may be introduced to a biological particle by using a vector system, such as a retroviral (e.g., lentiviral) vector system or a plasmid. The vector system may include a cassette encoding various components including a gRNA sequence (or antisense gRNA sequence), a barcode sequence (e.g., guide barcode sequence), and one or more primer sequences or partial primer sequences (or adapter sequences) or complements thereof. The one or more primer sequences (or adapter sequences) may be disposed adjacent to (e.g., upstream or downstream of) the gRNA sequence and/or barcode sequence. In some instances, the cassette comprises a promoter sequence (e.g., U6 promoter) that is upstream of the gRNA and barcode sequences. In some instances, e.g., when using a retroviral vector system, integration of the cassette into one or more DNA molecules of the biological particle (e.g., the genome of a cell) may occur. Transcription of the cassette or a portion thereof (e.g., via a RNA polymerase II) may result in a transcript comprising the gRNA sequence, the barcode sequence, and the one or more primer sequences (or adapter sequences), or complements thereof. The transcript may comprise or be a part of a gRNA molecule, as described herein. Additional methods and systems for generating vector cassettes may include, for example, CRISPR droplet sequencing (CROP-seq) and/or Perturb-Seq.

Additional nucleic acid molecules (e.g., primers, capture sequences) may be added to the biological particle, which may couple to a portion of the gRNA molecule (or precursor or derivative thereof). For example, a gRNA molecule in a biological particle (e.g., provided to a biological particle or produced therein, as described herein) may be contacted with one or more primers or capture nucleic acid molecules within the biological particle (e.g., cell, cell bead, or cell nucleus). In another example, a gRNA molecule, a precursor molecule (e.g., a vector cassette comprising a gRNA sequence or a portion thereof which may be used to generate the gRNA molecule), or a derivative of the gRNA molecule (e.g., a complementary DNA molecule generated from the gRNA molecule), optionally comprising a primer sequence and/or a barcode sequence (e.g., a gRNA barcode sequence that identifies the gRNA molecule) may be contacted with a primer molecule within a biological particle (e.g., a permeabilized biological particle). The primer molecule may comprise a primer sequence, which primer sequence may be a targeted primer sequence or a non-specific primer sequence (e.g., random N-mer). A targeted primer sequence may comprise, for example, a polyT sequence, which polyT sequence may interact with a polyA sequence of a gRNA molecule, a gRNA-containing transcript (e.g., CROP-seq), or a guide barcode construct (e.g., Perturb-seq) associated with a particular gRNA. Alternatively or in addition, a targeted primer sequence may comprise a sequence complementary to all or a portion of a sequence of a gRNA molecule, such as a crRNA or tracrRNA sequence of a gRNA molecule, or a precursor or derivative thereof. In some instances, the gRNA molecules across a plurality of biological particles may comprise a common primer sequence; accordingly, the primer sequence of the primer molecule (which may be targeted or random) may be used in multiple biological particles comprising different gRNA sequences that have the common primer sequence.

In some embodiments, the processing of a gRNA molecule (e.g., a gRNA molecule produced within a cell), a precursor of the gRNA molecule (e.g., a cassette vector comprising a gRNA sequence), a portion of the gRNA molecule or precursor, or a derivative of the gRNA molecule (e.g., a complementary DNA of the gRNA molecule), any of which may be associated with the gRNA or a gRNA-identifying barcode from the vector, may or may not include a reverse transcription step. In some examples, a gRNA molecule (e.g., a gRNA molecule produced within a cell) or an mRNA molecule may be contacted with a reverse transcriptase to generate a complementary DNA. In some cases, a gRNA-identifying barcode comprised by an mRNA molecule may be contacted with a reverse transcriptase to generate a complementary DNA. In some aspects, reverse transcription is performed prior to contacting the gRNA molecule with the first and second primer molecules. In some cases, a sequence comprised by a precursor of the gRNA molecule (e.g., a cassette vector comprising a gRNA sequence and/or a gRNA-identifying barcode) may be amplified (e.g., directly off the vector) using first and second primer molecules from the vector without performing a reverse transcription step.

In some instances, the additional nucleic acid molecules comprise a primer molecule that may couple to a gRNA molecule, a precursor molecule (e.g., a vector cassette comprising a gRNA sequence), or a derivative of the gRNA molecule (e.g., a cDNA molecule generated from the gRNA molecule). A primer molecule (e.g., primer nucleic acid molecule) may comprise one or more additional sequences, such as one or more sample index sequences, spacer or linkers sequences, or one or more additional primer sequences. In some examples, an extension reaction is performed to append a functional sequence, e.g., a sequencing primer sequence or portion thereof (e.g., an R1 or R2 sequence or portion thereof), or a complement of any of these sequences, to the sequence of the gRNA molecule (or precursor or derivative thereof). In some instances, the vector comprising a gRNA sequence does not comprise an integrated sequencing primer (e.g., a Nextera R1 or R2 adapter). Upon contacting and, e.g., hybridizing to a gRNA molecule (or precursor or derivative thereof), a primer extension reaction may be performed to provide an extended primer molecule having a sequence complementary to a sequence of the gRNA molecule (or precursor or derivative thereof). In some examples, an extension product is formed comprising the primer molecule and the gRNA molecule (or precursor or derivative thereof). In some embodiments, a second primer molecule is contacted with the extension product to generate a second extension product. In some cases, multiple primer molecules may contact the same gRNA molecules (or precursors or derivatives thereof). For example, a first primer molecule may contact a gRNA molecule at a first end of the gRNA molecule and a second primer molecule may contact the gRNA molecule at a second end of the gRNA molecule, which second end is opposite the first end. Primer extension may be performed in each direction to generate two complementary copies of the gRNA molecule comprising sequences of the primer molecule, which sequences may include functional sequences for subsequent analysis and processing (e.g., as described herein). Primer molecules that are configured to contact a gRNA molecule (or precursor or derivative thereof) at opposite ends may be primer pairs (e.g., a forward primer and a reverse primer). Contacting a gRNA molecule (or precursor or derivative thereof) with one or more primer molecules may be performed in a bulk solution. Alternatively, contacting a gRNA molecule (or precursor or derivative thereof) with one or more primer molecules may be performed within a partition (e.g., droplet or well). In some embodiments, the extension product is generated using said vector as template. In some embodiments, the extension product is generated using an mRNA that is transcribed from the vector as template. For example, a first primer molecule may contact the vector to generate the first extension product. In some examples, a first primer molecule may contact the mRNA that is transcribed from the vector and use the mRNA as template to generate the first extension product. In some aspects, the extension product generated comprises a gRNA-containing transcript (e.g., CROP-seq), or a guide barcode construct (e.g., Perturb-seq) associated with a particular gRNA.

The methods provided herein may comprise contacting a biological particle (e.g., a cell, cell bead, or cell nucleus) with a transposase-nucleic acid complex comprising a transposase molecule and one or more transposon end oligonucleotide molecules. The biological particle may be contacted with a transposase-nucleic acid complex in bulk solution, such that the biological particle undergoes “tagmentation” via a tagmentation reaction. Contacting the biological particle with a transposase-nucleic acid complex may generate one or more template nucleic acid fragments (e.g., “tagmented fragments”). The one or more template nucleic acid fragments may correspond to one or more target nucleic acid molecules (e.g., DNA molecules) within the biological particle.

A nucleic acid molecule may undergo one or more processing steps within a biological particle (e.g., cell, cell bead, or cell nucleus). For example, chromatin within a cell, cell bead, or cell nucleus may be contacted with a transposase. Examples of processing steps including a transposon complex may be found in, for example, US. Pat. Pub. 20180340171 and US. Pat. Pub. 20200291454; each of which are herein incorporated by reference in their entireties.

A transposase may be included within a transposase-nucleic acid complex, which transposase-nucleic acid complex may comprise a transposase molecule and one or more transposon end oligonucleotide molecules. A transposase may be a Tn transposase, such as a Tn3, Tn5, Tn7, Tn10, Tn552, Tn903 transposase. Alternatively, a transposase may be a MuA transposase, a Vibhar transposase (e.g. from Vibrio harveyi), Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Te1, THE-1, Tn/O, TnA, Tn3, Tn5, Tn7, Tn10, Tn552, Tn903, Tol1, Tol2, TnIO, Tyl, any prokaryotic transposase, or any transposase related to and/or derived from those listed above. For example, a transposase may be a Tn5 transposase or a mutated, hyperactive Tn5 transposase. A transposase related to and/or derived from a parent transposase may comprise a peptide fragment with at least about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% amino acid sequence homology to a corresponding peptide fragment of the parent transposase. The peptide fragment may be at least about 10, about 15, about 20, about 25, about 30, about 35, about 40, about 45, about 50, about 60, about 70, about 80, about 90, about 100, about 150, about 200, about 250, about 300, about 400, or about 500 amino acids in length. For example, a transposase derived from Tn5 may comprise a peptide fragment that is 50 amino acids in length and about 80% homologous to a corresponding fragment in a parent Tn5 transposase. Action of a transposase (e.g., insertion) may be facilitated and/or triggered by addition of one or more cations, such as one or more divalent cations (e.g., Ca²⁺, Mg²⁺, or Mn²⁺).

A transposase-nucleic acid complex may comprise one or more nucleic acid molecules. For example, a transposase-nucleic acid complex may comprise one or more transposon end oligonucleotide molecules. A transposon end oligonucleotide molecule may comprise one or more adapter sequences (e.g., comprising one or more primer sequences) and/or one or more transposon end sequences. A transposon end sequence may be, for example, a Tn5 or modified Tn5 transposon end sequence or a Mu transposon end sequence. A transposon end sequence may have a sequence of, for example,

(SEQ ID NO: 1)
AGATGTGTATAAGAGACA.

A primer sequence of a transposon end oligonucleotide molecule may be a sequencing primer, such as an R1 or R2 sequencing primer, or a portion thereof. A sequencing primer may be, for example, a TrueSeq or Nextera sequencing primer. An R1 sequencing primer region may have a sequence of

(SEQ ID NO: 2)
TCTACACTCTTTCCCTACACGACGCTCTTCCGATCT,

or some portion thereof. An R1 sequencing primer region may have a sequence of

(SEQ ID NO: 3)
TCGTCGGCAGCGTCAGATGTGTATAAGAGACAG,

or some portion thereof. A transposon end oligonucleotide molecule may comprise a partial R1 sequence. A partial R1 sequence may be

(SEQ ID NO: 4)
ACTACACGACGCTCTTCCGATCT.

A transposon end oligonucleotide molecule may comprise an R2 sequencing priming region. An R2 sequencing primer region may have a sequence of

(SEQ ID NO: 5)
GTGACTGGAGTTCAGACGTGTGCTCTTCCGATCT,

or some portion thereof. An R2 sequencing primer region may have a sequence of

(SEQ ID NO: 6)
GTCTCGTGGGCTCGGAGATGTGTATAAGAGACAG,

or some portion thereof. A transposon end oligonucleotide molecule may comprise a T7 promoter sequence. A T7 promoter sequence may be

(SEQ ID NO: 7)
TAATACGACTCACTATAG.

A transposon end oligonucleotide molecule may comprise a region at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NO: 1-7. A transposon end oligonucleotide molecule may comprise a P5 sequence and/or a P7 sequence. A transposon end oligonucleotide molecule may comprise a sample index sequence, such as a barcode sequence or unique molecular identifier sequence. One or more transposon end oligonucleotide molecules of a transposase-nucleic acid complex may be attached to a solid support (e.g., a solid or semi-solid particle such as a bead (e.g., gel bead)). A transposon end oligonucleotide molecule may be releasably coupled to a solid support (e.g., a bead). Examples of transposon end oligonucleotide molecules may be found in, for example, PCT Patent Publications Nos. WO2018/218226, WO2014/189957, US. Pat. Pub. 20180340171, US. Pat. Pub. 20200291454, U.S. Pat. Nos. 10,400,235, and 10,059,989; each of which are herein incorporated by reference in their entireties.

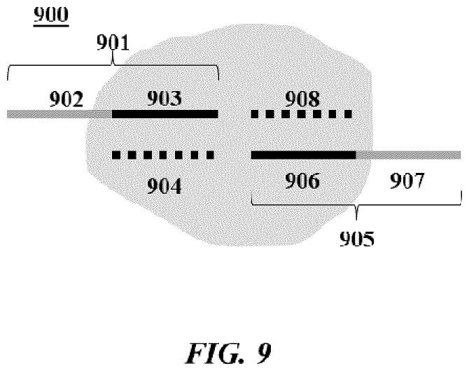

FIG. 9 includes an example of a transposase-nucleic acid complex for use in the methods provided herein. Transposase-nucleic acid complex 900 (e.g., comprising a transpose dimer) comprises partially double-stranded oligonucleotide 901 and partially double-stranded oligonucleotide 905. Partially double-stranded oligonucleotide 901 comprises transposon end sequence 903, first primer sequence 902, and a sequence 904 that is complementary to transposon end sequence 903. Partially double-stranded oligonucleotide 905 comprises transposon end sequence 906, first primer sequence 907, and a sequence 908 that is complementary to transposon end sequence 906. Primer sequences 902 and 907 may be the same or different. In some cases, primer sequence 902 may be designated as a first sequencing primer, such as an Illumina “R1” sequence and primer sequence 907 may be designated as a second sequencing primer, such as an Illumina “R2” sequence. Transposon end sequences 903 and 906 may be the same or different.

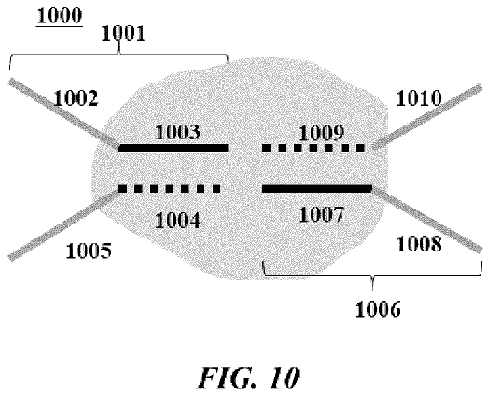

FIG. 10 includes another example of a transposase-nucleic acid complex for use in the methods provided herein. Transposase-nucleic acid complex 1000 (e.g., comprising a transpose dimer) comprises forked adapters 1001 and 1006, which forked adapters are partially double-stranded oligonucleotides. Partially double-stranded oligonucleotide 1001 comprises transposon end sequence 1003, first primer sequence 1002, second primer sequence 1005, and a sequence 1004 that is complementary to transposon end sequence 1003. Partially double-stranded oligonucleotide 1006 comprises transposon end sequence 1007, first primer sequence 1008, second primer sequence 1010, and a sequence 1009 that is complementary to transposon end sequence 1007. Primer sequences 1002, 1005, 1008, and 1010 may be the same or different. In some cases, primer sequences 1002 and 1008 may be designated as a first sequencing primer, such as an Illumina “R1” sequence and primer sequences 1005 and 1010 may be designated as a second sequencing primer, such as an Illumina “R2” sequence. Alternatively, primer sequences 1002 and 1010 may be designated “R1” and primer sequences 1005 and 1008 may be designated “R2”. Alternatively, primer sequences 1002 and 1008 may be designated “R2” and primer sequences 1005 and 1010 may be designated “R1”. Alternatively, primer sequences 1002 and 1010 may be designated “R2” and primer sequences 1005 and 1008 may be designated “R1”. Transposon end sequences 1003 and 1007 may be the same or different.

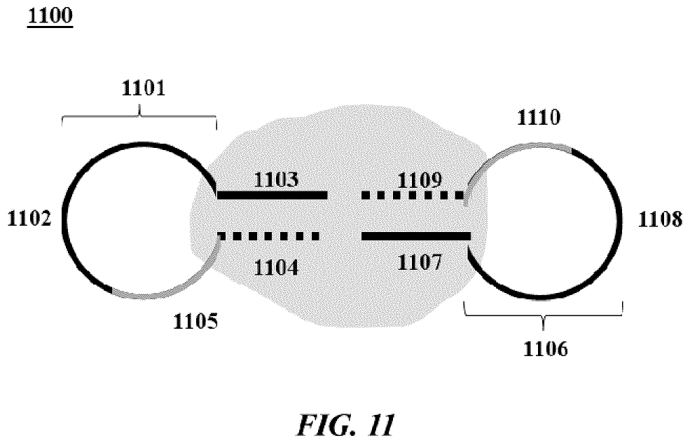

FIG. 11 shows transposase-nucleic acid complex 1100 (e.g., comprising a transpose dimer) comprising hairpin molecules 1101 and 1106. Hairpin molecule 1101 comprises transposon end sequence 1103, first hairpin sequence 1102, second hairpin sequence 1105, and a sequence 1104 that is complementary to transposon end sequence 1103. Hairpin molecule 1106 comprises transposon end sequence 1107, third hairpin sequence 1108, fourth hairpin sequence 1110, and a sequence 1109 that is complementary to transposon end sequence 1107. Hairpin sequences 1102, 1105, 1108, and 1110 may be the same or different. For example, hairpin sequence 1105 may be the same or different as hairpin sequence 1110, and/or hairpin sequence 1102 may be the same or different as hairpin sequence 1108. Hairpin sequences 1102 and 1108 may be spacer sequences or adapter sequences. Hairpin sequences 1105 and 1110 may be a promoter sequence such as T7 recognition or promoter sequences and/or UMI sequences. Transposon end sequences 1103 and 1107 may be the same or different. In some cases, sequence 1104 is a transposon end sequence and 1103 is a sequence complementary to sequence 1104. In some cases, sequence 1109 is a transposon end sequence and 1107 is a sequence complementary to sequence 1109.

Contacting a biological particle (e.g., cell, cell bead, or cell nucleus) comprising one or more target nucleic acid molecules (e.g., DNA molecules) with a transposase-nucleic acid complex may generate one or more tagged (see, e.g., FIGS. 9-10) template nucleic acid fragments (e.g., “tagmented fragments”). The one or more template nucleic acid fragments may each comprise a sequence of the one or more target nucleic acid molecules (e.g., a target sequence). The transposase-nucleic acid complex may be configured to target a specific region of the one or more target nucleic acid molecules (e.g., conjugated to an antibody specific for a protein bound to the target sequence) to provide one or more template nucleic acid fragments comprising specific target sequences. The one or more template nucleic acid fragments may comprise target sequences corresponding to accessible chromatin. Generation of tagmented fragments may take place within a bulk solution. In other cases, generation of tagmented fragments may take place within a partition (e.g., a droplet or well). A template nucleic acid fragment (e.g., tagmented fragment) may comprise one or more gaps (e.g., between a transposon end sequence or complement thereof and a target sequence on one or both strands of a double-stranded fragment). Gaps may be filled via a gap filling process using an enzyme, e.g., a polymerase (e.g., DNA polymerase). In some cases, a mixture of enzymes may be used to repair a partially double-stranded nucleic acid molecule and fill one or more gaps. Gap filling may not include strand displacement. Gaps may be filled within or outside of a partition.

Processing of nucleic acid molecules within a biological particle (e.g., cell, cell bead, or cell nucleus) (e.g., generation of template nucleic acid fragments using a transposase-nucleic acid complex (e.g., ATAC-seq) and/or generation of additional template nucleic acid fragments corresponding to a template gRNA molecule or derivative thereof using a capture nucleic acid molecule such a primer molecule) may occur in a bulk solution comprising a plurality of biological particles (e.g., cells, cell beads, and/or cell nuclei). In some cases, template nucleic acid fragments (e.g., tagmented fragments) may be generated in a partition. In some cases, template nucleic acid fragments (e.g., tagmented fragments) may be generated in bulk solution and additional template nucleic acid fragments (e.g., other DNA and/or RNA fragments) may be generated in a partition. In some cases, template nucleic acid fragments (e.g., tagmented fragments) may be generated prior to providing a partition comprising a biological particle.

A plurality of biological particles (e.g., cells, cell beads, and/or cell nuclei, such as a plurality of cells, cell beads, and/or cell nuclei that have undergone processing such as a tagmentation process) may be partitioned amongst a plurality of partitions. Partitions may be, for example, droplets or wells. Droplets (e.g., aqueous droplets) may be generated according to the methods provided herein. Partitioning may be performed according to the methods provided herein. For example, partitioning a biological particle (e.g., cell, cell bead, or cell nucleus) and one or more reagents may comprise flowing a first phase comprising an aqueous fluid, the biological particle, and the one or more reagents and contacting a second phase comprising a fluid that is immiscible with the aqueous fluid. Upon interaction of the first and second phases, a discrete droplet of the first phase comprising the biological particle and the one or more reagents may be formed. The plurality of biological particles (e.g., cells, cell beads, and/or cell nuclei) may be partitioned amongst a plurality of partitions such that at least a subset of the plurality of partitions may comprise at most one cell, cell bead, or cell nucleus. Biological particles (e.g., cells, cell beads, and/or cell nuclei) may be co-partitioned with one or more reagents such that a partition of at least a subset of the plurality of partitions comprises a single biological particle (e.g., cell, cell bead, or cell nucleus) and one or more reagents. The one or more reagents may include, for example, enzymes (e.g., polymerases, reverse transcriptases, ligases, etc.), nucleic acid barcode molecules (e.g., nucleic acid barcode molecules comprising one or more barcode sequences, such as nucleic acid barcode molecules coupled to one or more beads), template switching oligonucleotides, deoxynucleotide triphosphates, buffers, lysis agents, primers, barcodes, detergents, reducing agents, chelating agents, oxidizing agents, nanoparticles, beads, antibodies, or any other useful reagents. Enzymes may include, for example, temperature-sensitive enzymes, pH-sensitive enzymes, light-sensitive enzymes, reverse transcriptases, proteases, ligases, polymerases, kinases, restriction enzymes, nucleases, protease inhibitors, exonucleases, and nuclease inhibitors.

A reagent of the one or more reagents may be useful for lysing or permeabilizing a biological particle (e.g., cell, cell bead, or cell nucleus), or otherwise providing access to nucleic acid molecules and/or template nucleic acid fragments therein. A cell may be lysed using a lysis agent such as a bioactive agent. A bioactive agent useful for lysing a cell may be, for example, an enzyme (e.g., as described herein). An enzyme used to lyse a cell may or may not be capable of carrying out additional actions such as degrading one or more RNA molecules. Alternatively, an ionic, zwitterionic, or non-ionic surfactant may be used to lyse a cell. Examples of surfactants include, but are not limited to, TritonX-100, Tween 20, sarcosyl, or sodium dodecyl sulfate. Cell lysis may also be achieved using a cellular disruption method such as an electroporation or a thermal, acoustic, or mechanical disruption method. Alternatively, a cell may be permeabilized to provide access to a plurality of nucleic acid molecules included therein. Permeabilization may involve partially or completely dissolving or disrupting a cell membrane or a portion thereof. Permeabilization may be achieved by, for example, contacting a cell membrane with an organic solvent or a detergent such as Triton X-100 or NP-40. By lysing or permeabilizing a biological particle (e.g., cell, cell bead, or cell nucleus) within a partition (e.g., droplet) to provide access to the plurality of nucleic acid molecules and/or template nucleic acid fragments therein, molecules originating from the same biological particle (e.g., cell, cell bead, or cell nucleus) may be isolated within the same partition.

A partition of a plurality of partitions (e.g., a partition comprising a cell, cell bead, and/or cell nucleus) may comprise one or more supports. In some aspects, the supports may comprise beads (e.g., gel beads). A bead may be a gel bead. A bead may comprise a plurality of nucleic acid barcode molecules (e.g., nucleic acid molecules each comprising one or more barcode sequences, as described herein). A bead may comprise at least 10,000 nucleic acid barcode molecules attached thereto. For example, the bead may comprise at least 100,000, 1,000,000, or 10,000,000 nucleic acid barcode molecules attached thereto. The plurality of nucleic acid barcode molecules may be releasably attached to the bead. The plurality of nucleic acid barcode molecules may be releasable from the bead upon application of a stimulus. Such a stimulus may be selected from the group consisting of a thermal stimulus, a photo stimulus, and a chemical stimulus. For example, the stimulus may be a reducing agent such as dithiothreitol Application of a stimulus may result in one or more of (i) cleavage of a linkage between nucleic acid barcode molecules of the plurality of nucleic acid barcode molecules and the bead, and (ii) degradation or dissolution of the bead to release nucleic acid barcode molecules of the plurality of nucleic acid barcode molecules from the bead. In some cases, the plurality of nucleic acid barcode molecules may be attached to the bead via a plurality of labile moieties, such as a plurality of labile bonds. Examples of labile bonds include, for example, an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). In some cases, the plurality of nucleic acid barcode molecules may be attached to the bead via thermally cleavable bonds, disulfide bonds, or UV sensitive bonds.

A plurality of nucleic acid barcode molecules attached (e.g., releasably attached) to a support (e.g., a bead or gel bead) may be suitable for barcoding template nucleic acid fragments (e.g., tagmented fragments, as described herein) or a gRNA molecule or derivative thereof of the plurality of biological particles (e.g., cells, cell beads, and/or cell nuclei). For example, a nucleic acid barcode molecule of a plurality of nucleic acid barcode molecule may comprise a barcode sequence, unique molecular identifier (UMI) sequence, primer sequence, universal primer sequence, sequencing adapter or primer, flow cell adapter sequence, or any other useful feature. In an example, a nucleic acid barcode molecule of a plurality of nucleic acid barcode molecules attached to a support (e.g., a bead) may comprise a flow cell adapter sequence (e.g., a P5 or P7 sequence), a barcode sequence, and a sequencing primer sequence or portion thereof (e.g., an R1 or R2 sequence or portion thereof), or a complement of any of these sequences. These sequences may be arranged in any useful order and may be linked or may include one or more spacer sequences disposed between them. For instance, the flow cell adapter sequence, where present, may be disposed near (e.g., proximal to) an end of the nucleic acid barcode molecule that is closest to the bead, while the sequencing primer or portion thereof may be disposed at an end of the nucleic acid barcode molecule that is furthest from (e.g., distal to) the bead (e.g., most available to template nucleic acid fragments for interaction). In another example, a nucleic acid barcode molecule of a plurality of nucleic acid barcode molecules attached to a support (e.g., a bead) may comprise a flow cell adapter sequence (e.g., a P5 or P7 sequence), a barcode sequence, a sequencing primer sequence or portion thereof (e.g., an R1 or R2 sequence or portion thereof), and a UMI sequence, or a complement of any of these sequences. The nucleic acid barcode molecule may further comprise a capture sequence, which capture sequence may be a targeted capture sequence or comprise a template switch sequence (e.g., comprising a polyC or poly G sequence). These sequences may be arranged in any useful order and may be linked or may include one or more spacer sequences disposed between them. For instance, the flow cell adapter sequence may be disposed near (e.g., proximal to) an end of the nucleic acid barcode molecule that is closest to the bead, while the capture sequence or template switch sequence may be disposed at an end of the nucleic acid barcode molecule that is furthest from the bead (e.g., most available to template nucleic acid fragments for interaction).

All of the nucleic acid barcode molecules attached (e.g., releasably attached) to a support (e.g., a bead or gel bead) of a plurality of beads may be the same. For example, all of the nucleic acid barcode molecules attached to the bead may have the same nucleic acid sequence. In such an instance, all of the nucleic acid barcode molecules attached to the bead may comprise the same flow cell adapter sequence, sequencing primer or portion thereof, and/or barcode sequence. The barcode sequence of a plurality of nucleic acid barcode molecules attached to a bead of a plurality of beads may be different from other barcode sequences of other nucleic acid barcode molecules attached to other beads of the plurality of beads. For example, a plurality of beads may comprise a plurality of barcode sequences, such that, for at least a subset of the plurality of beads, each bead comprises a different barcode sequence of the plurality of barcode sequences. This differentiation may permit template nucleic acid fragments (e.g., included within cells, cell beads, and/or cell nuclei) co-partitioned with a plurality of beads between a plurality of partitions to be differentially barcoded within their respective partitions, such that the template nucleic acid fragments or molecules derived therefrom may be identified with the partition (and thus the cell, cell bead, and/or cell nucleus) to which they correspond (e.g., using a nucleic acid sequencing assay, as described herein). A barcode sequence may comprise between, e.g., 4-20 nucleotides. A barcode sequence may comprise one or more segments, which segments may range in size from 2-20 nucleotides, such as from 4-20 nucleotides. Such segments may be combined to form barcode sequences using a combinatorial assembly method, such as a split-pool method. Details of such methods can be found, for example, in PCT/US2018/061391, filed Nov. 15, 2018, and U.S. Pat. Pub. 20190249226, each of which are herein incorporated by reference in their entireties.

In some cases, nucleic acid barcode molecules attached to a support (e.g., a bead) may not be the same. For example, the plurality of nucleic acid barcode molecules attached to a bead may each comprise a UMI sequence, which UMI sequence varies across the plurality of nucleic acid barcode molecules. All other sequences of the plurality of nucleic acid barcode molecules attached to the bead may be the same.

In some cases, a support (e.g., a bead) may comprise multiple different nucleic acid barcode molecules attached thereto. For example, a bead may comprise a first plurality of nucleic acid barcode molecules and a second plurality of nucleic acid barcode molecules, which first plurality of nucleic acid barcode molecules is different than the second plurality of nucleic acid barcode molecules. The first plurality of nucleic acid barcode molecules and the second plurality of nucleic acid barcode molecules coupled to a bead may comprise one or more shared sequences. For example, each nucleic acid barcode molecule of the first plurality of nucleic acid barcode molecules and each nucleic acid barcode molecule of the second plurality of nucleic acid barcode molecules may comprise the same barcode sequence (e.g., as described herein). Such a barcode sequence may be prepared using a combinatorial assembly process (e.g., as described herein). For example, barcode sequences may comprise identical barcode sequence segments. Similarly, each nucleic acid barcode molecule of the first plurality of nucleic acid barcode molecules coupled to a bead may comprise the same flow cell adapter sequence and/or sequencing primer or portion thereof as each nucleic acid barcode molecule of the second plurality of nucleic acid barcode molecules coupled to the bead. In an example, each nucleic acid barcode molecule of the first plurality of nucleic acid barcode molecules coupled to a bead comprises a sequencing primer, and each nucleic acid barcode molecule of the second plurality of nucleic acid barcode molecules coupled to the bead comprises a portion of the same sequencing primer. In some instances, each nucleic acid barcode molecule of the first plurality of nucleic acid barcode molecules coupled to a bead may comprise a first sequencing primer (e.g., a TruSeq R1 sequence), a barcode sequence, and a first functional sequence, and each nucleic acid barcode molecule of the second plurality of nucleic acid barcode molecules coupled to the bead may comprise a second sequencing primer (e.g., a Nextera R1 sequence, or a portion thereof), the barcode sequence, and a second functional sequence. Sequences shared between different sets of nucleic acid barcode molecules coupled to the same bead may be included in the same or different order and may be separated by the same or different sequences. Alternatively or in addition, the first plurality of nucleic acid barcode molecules and the second plurality of nucleic acid barcode molecules coupled to a bead may include one or more different sequences. For example, each nucleic acid barcode molecule of a first plurality of nucleic acid barcode molecules coupled to a bead of a plurality of beads may comprise one or more of a flow cell adapter sequence, a barcode sequence, UMI sequence, capture sequence, and a sequencing primer or portion thereof, while each nucleic acid barcode molecule of a second plurality of nucleic acid barcode molecules coupled to the bead may comprise one or more of a flow cell adapter sequence (e.g., the same flow cell adapter sequence), a barcode sequence (e.g., the same barcode sequence), UMI sequence, capture sequence, and a sequencing primer or portion thereof (e.g., the same sequencing primer or portion thereof). Nucleic acid barcode molecules of the first plurality of nucleic acid barcode molecules may not include a UMI sequence or capture sequence. A bead comprising multiple different populations of nucleic acid barcode molecules, such as a first plurality of nucleic acid molecules and a second plurality of nucleic acid molecules (e.g., as described above), may be referred to as a “multi-functional bead.”

In some embodiments, additional analytes may be processed. For example, a biological particle (e.g., cell, cell bead, or cell nucleus) comprising template nucleic acid fragments (e.g., template nucleic acid fragments and additional template nucleic acid fragments deriving from DNA or RNA molecules included within the biological particle) may be co-partitioned with one or more beads (e.g., as described herein). For example, a biological particle (e.g., cell, cell bead, or cell nucleus) may be co-partitioned with a first bead (e.g., first gel bead) configured to interact with a first set of template nucleic acid fragments (e.g., template nucleic acid fragments deriving from DNA molecules, such as tagmented fragments and/or other nucleic acid molecules e.g., perturbation agents) and a second bead (e.g., second gel bead) configured to interact with an additional set of template nucleic acid fragments (e.g., additional template nucleic acid fragments deriving from RNA molecules). The first and second beads may each comprise a plurality of nucleic acid molecules. For example, the first bead may comprise a plurality of first nucleic acid molecules and the second bead may comprise a plurality of second nucleic acid molecules, where each first nucleic acid molecule of the plurality of first nucleic acid molecules comprises a first shared sequence and each second nucleic acid molecule of the plurality of second nucleic acid molecules comprises a second shared sequence. The first shared sequence and the second shared sequence may be the same or different. The first shared sequence and the second shared sequence may comprise one or more shared components, such as a shared barcode sequence or sequencing primer or portion thereof.

A nucleic acid barcode molecule of the first plurality of nucleic acid barcode molecules may comprise a flow cell adapter sequence, a barcode sequence, and a sequencing primer or portion thereof, which sequencing primer or portion thereof may be configured to interact with (e.g., anneal or hybridize to) a complementary sequence included in template nucleic acid fragments deriving from DNA molecules of the biological particle (e.g., cell, cell bead, or cell nucleus), or derivatives thereof (e.g., ATAC-seq fragments generated using, e.g., the composition of FIGS. 9-11). A nucleic acid barcode molecule of the second plurality of nucleic acid barcode molecules may comprise the flow cell adapter sequence, the barcode sequence, the sequencing primer or a portion thereof, a UMI sequence, and a capture sequence, which capture sequence may be configured to interact with (e.g., anneal or hybridize to) a sequence of template nucleic acid fragments deriving from nucleic acid molecules (e.g., RNA molecules) of the biological particle (e.g., cell, cell bead, or cell nucleus), or derivatives thereof. The first plurality of nucleic acid barcode molecules may comprise approximately the same number of nucleic acid barcode molecules as the second plurality of nucleic acid barcode molecules. Alternatively, the first plurality of nucleic acid barcode molecules may comprise a greater number of nucleic acid barcode molecules than the second plurality of nucleic acid barcode molecules, or vice versa. The distribution of nucleic acid barcode molecules on a support (e.g., a bead) may be controlled by, for example, sequence control, concentration control, and or blocking methods during assembly of the nucleic acid barcode molecules on the bead. Details of such processes are provided in, for example, PCT/US2018/061391, filed Nov. 15, 2018, U.S. Pat. Pub. 20190249226, US. Pat. Pub. 20180340171, and US. Pat. Pub. 20200291454, each of which are incorporated by reference in their entireties.

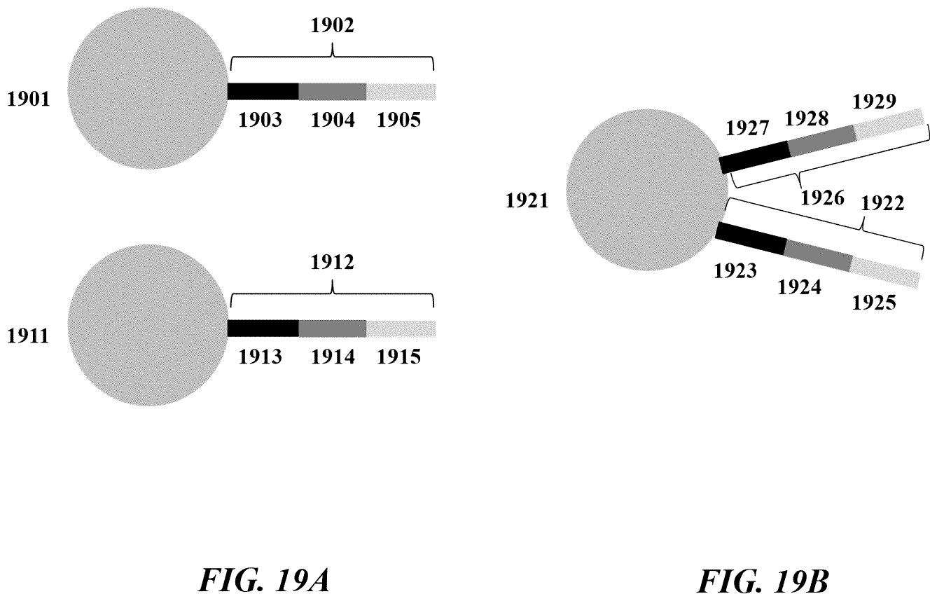

FIGS. 19A and 19B show examples of beads for use according to the method provided herein. FIG. 19A shows a first bead 1901 and a second bead 1911 that may be co-partitioned with a biological particle (e.g., cell, cell bead, or cell nucleus) into a partition of a plurality of partitions (e.g., droplets or wells). First bead 1901 may comprise nucleic acid molecule 1902. Nucleic acid molecule 1902 may comprise sequences 1903, 1904, and 1905. Sequence 1903 may be, for example, a flow cell adapter sequence (e.g., a P5 or P7 sequence). Sequence 1904 may be, for example, a barcode sequence. Sequence 1905 may be, for example, a sequencing primer sequence or portion thereof (e.g., an R1 or R2 primer sequence, or portion thereof). Nucleic acid molecule 1902 may also include additional sequences, such as a UMI sequence. First bead 1901 may comprise a plurality of nucleic acid molecules 1902. Second bead 1911 may comprise nucleic acid molecule 1912. Nucleic acid molecule 1912 may comprise sequences 1913, 1914, and 1915. Sequence 1913 may be, for example, a flow cell adapter sequence (e.g., a P5 or P7 sequence). Sequence 1914 may be, for example, a barcode sequence. Sequence 1915 may be, for example, a sequencing primer sequence or portion thereof (e.g., an R1 or R2 primer sequence, or portion thereof). Nucleic acid molecule 1912 may also include additional sequences, such as a UMI sequence and a capture sequence. Second bead 1911 may comprise a plurality of nucleic acid molecules 1912.

FIG. 19B shows a bead 1921 (e.g., a multifunctional bead having two or more species of nucleic acid barcode molecules attached or coupled thereto) that may be co-partitioned with a biological particle (e.g., cell, cell bead, or cell nucleus) into a partition of a plurality of partitions (e.g., droplets or wells). Bead 1921 may comprise nucleic acid molecule 1922 and nucleic acid molecule 1926. Nucleic acid molecule 1922 may comprise sequences 1923, 1924, and 1925. Sequence 1923 may be, for example, a flow cell adapter sequence (e.g., a P5 or P7 sequence). Sequence 1924 may be, for example, a barcode sequence. Sequence 1925 may be, for example, a sequencing primer or portion thereof (e.g., an R1 or R2 primer sequence, or portion thereof, such as a Nextera R1 sequence or portion thereof). In some instances, sequence 1925 may also be, for example, a sequence configured to hybridize to a splint oligonucleotide as described elsewhere herein. Nucleic acid molecule 1926 may comprise sequences 1927, 1928, and 1929. Sequence 1927 may be, for example, a flow cell adapter sequence (e.g., a P5 or P7 sequence). Sequence 1928 may be, for example, a barcode sequence (e.g., the same barcode sequence as sequence 1924). Sequence 1929 may be, for example, a sequencing primer or portion thereof (e.g., an R1 or R2 primer sequence, or portion thereof). Sequence 1927 may be, for example, a sequencing primer or portion thereof (e.g., an R1 or R2 primer sequence, or portion thereof, such as a TruSeq R1 sequence, or portion thereof). Sequence 1928 may be, for example, a barcode sequence (e.g., the same barcode sequence as 1924). Sequence 1929 may be, for example, a capture sequence (e.g., a poly-T sequence), such as a capture sequence that is configured to hybridize with or ligate to a target nucleic acid molecule (e.g., gRNA molecule or derivative thereof, such as a primer extension or ligation product comprising an adapter sequence). Sequence 1929 may be, for example, a template switching oligonucleotide (TSO) sequence configured to facilitate a template switching reaction with a target nucleic acid molecule (e.g., RNA molecule). Sequence 1923 and sequence 1927 may be the same. Alternatively, sequence 1923 and sequence 1927 may be different. Sequence 1924 and sequence 1928 may be the same. Alternatively, sequence 1924 and sequence 1928 may be different. Sequence 1925 and sequence 1929 may be the same. Alternatively, sequence 1925 and sequence 1929 may be different. Nucleic acid molecules 1922 and 1926 may also include additional sequences, such as a UMI sequence and a capture sequence. Bead 1921 may comprise a plurality of nucleic acid molecules 1922 and a plurality of nucleic acid molecules 1926.

Following recovery of barcoded nucleic acid molecules corresponding to chromatin and gRNA molecules or derivatives thereof from partitions, and optional additional functionalization, barcoded nucleic acid molecules or derivatives thereof may be subjected to sorting, purification, and other additional processing. For example, barcoded nucleic acid molecules or derivatives thereof (e.g., chromatin and gRNA libraries) may be purified to select nucleic acid fragments of specific size ranges, having specific isoelectic properties, or having other biochemical or biophysical properties. For example, barcoded nucleic acid molecules or derivatives thereof may be purified by size using gel electrophoresis and selecting fragments of a desired size.

In some cases, the methods provided herein may comprise identification of a gRNA molecule or precursor thereof provided to a biological particle (e.g., cell, cell bead, or cell nucleus) via direct processing of a gRNA molecule or portion or derivative thereof (e.g., as described herein). In some cases, the methods provided herein may comprise identification of a gRNA molecule or precursor thereof provided to a particle (e.g., cell, cell bead, or cell nucleus) via processing or a gRNA-identifying barcode and/or direct processing of a gRNA molecule or portion or derivative thereof. In some cases, the methods provided herein may comprise assessing the presence or absence of a given gRNA-identifying barcode and/or the presence or absence of a sequence of a given gRNA molecule or derivative thereof (e.g., crRNA sequence). In some cases, cutoff's or threshold values may be assigned to assess the presence or absence of such sequences. For example, a number of reads (e.g., sequencing reads) associated with a given sequence may be counted and adjusted for sequencing depth (e.g., to account for library preparation or sequencing sensitivity variation). In another example, a minimum number of reads, such as 1,000 reads per biological particle, may be applied to remove sequences with low coverage. Cutoffs may also be used to remove sequences and/or reads from biological particles having high background reads, etc.

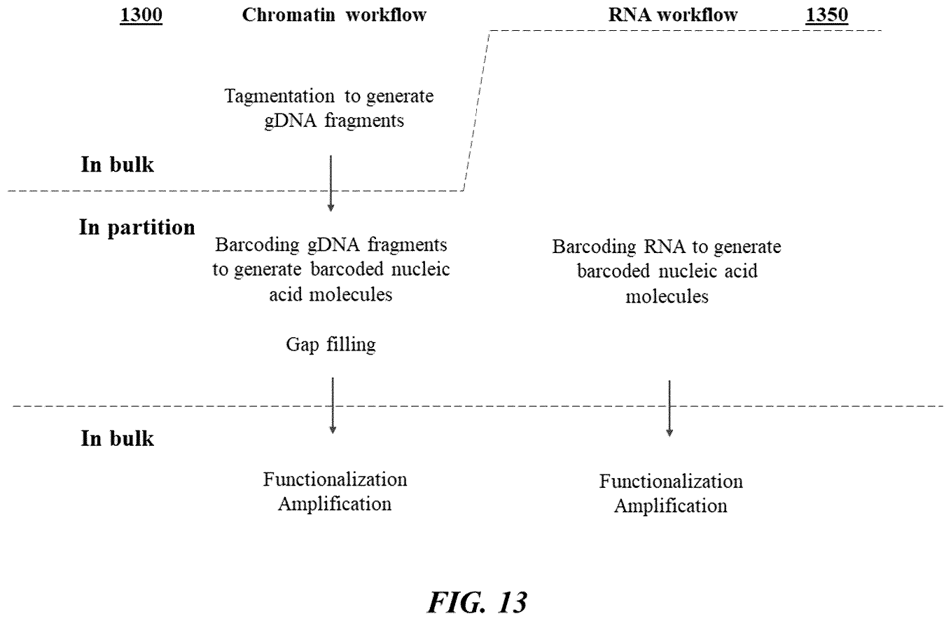

FIG. 13 includes a general workflow for methods provided herein. The left panel of the figure includes chromatin workflow 1300. Tagmentation may be performed in bulk to generate tagmented fragments of genomic DNA. The gDNA fragments may comprise one or more gaps (e.g., as described herein). A biological particle comprising one or more gDNA fragments may then be partitioned within a partition (e.g., droplet or well) with one or more reagents, including a first nucleic acid barcode molecule and a second nucleic acid barcode molecule (e.g., as described herein). One or more gDNA fragments of the partitioned biological particle and the first nucleic acid barcode molecule may be used to generate a first barcoded nucleic acid molecule. Gap filling may optionally be performed within the partition before or after generation of the first barcoded nucleic acid molecule. In some cases, gap filling may be performed prior to partitioning. In other cases, gap filling may be performed after recovery of the first barcoded nucleic acid molecule, or a derivative thereof, from the partition. Upon recovery from the partition, the first barcoded nucleic acid molecule, or a derivative thereof, may be subjected to additional processing to, e.g., incorporate additional functional groups including sequencing primers, flow cell adapters, identifying sequences, etc. and/or to amplify the barcoded nucleic acid molecule, or a derivative thereof, to enrich a sample population for subsequent analysis such as nucleic acid sequencing. The right panel of FIG. 13 includes RNA workflow 1350. A gRNA molecule or precursor thereof may be introduced to the biological particle prior to partitioning in the partition (e.g., droplet or well). Where a gRNA molecule precursor is introduced to the biological particle, the gRNA molecule may be produced within the biological particle via, for example, transcription of the precursor (e.g., as described herein). Following partitioning, the gRNA molecule and the second nucleic acid barcode molecule that may have the same or a different sequence as the first nucleic acid barcode molecule may be used to generate a second barcoded nucleic acid molecule. The second barcoded nucleic acid molecule, or a derivative thereof, may be recovered from the partition with the first barcoded nucleic acid molecule and optionally subjected to additional processing to, e.g., incorporate additional functional groups including sequencing primers, flow cell adapters, identifying sequences, etc. and/or to amplify the barcoded nucleic acid molecule, or a derivative thereof, to enrich a sample population for subsequent analysis such as nucleic acid sequencing. The first and second barcoded nucleic acid molecules, or derivatives thereof, may be separated from one another prior to undergoing such additional processing or may be processed in a common (e.g., bulk) solution. Similarly, the first and second barcoded nucleic acid molecules, or derivatives thereof, or amplicons thereof, may be subjected to nucleic acid sequencing at a same time and/or using a same system, or may be subjected to nucleic acid sequencing at a different time and/or using a different system.

Various schemes may be used to process a gRNA molecule or precursor or derivative thereof. In an example, a gRNA molecule (e.g., a gRNA molecule produced within a cell), a precursor of the gRNA molecule (e.g., a cassette vector comprising a gRNA sequence), a portion of the gRNA molecule or precursor, or a derivative of the gRNA molecule (e.g., a complementary DNA of the gRNA molecule), any of which may be associated with the gRNA or a gRNA-identifying barcode may be subjected to processing A gRNA molecule (or precursor or derivative thereof) within a biological particle (e.g., cell, cell bead, or cell nucleus) may be processed to provide a barcoded nucleic acid molecule. The biological particle may be disposed within a partition (e.g., a droplet or well) along with a bead comprising one or more nucleic acid barcode molecules (e.g., as described herein). A nucleic acid barcode molecule attached to the bead may be releasably attached from the bead and may be released from the bead (e.g., upon application of a stimulus and/or partial or complete degradation or dissolution of the bead within the partition). The nucleic acid barcode molecule may comprise a barcode sequence, a unique molecular identifier sequence, a flow cell adapter sequence or portion thereof, a capture or adapter sequence, or any other useful sequence, including a spacer sequence, arranged in any useful order. For example, the nucleic acid barcode molecule may comprise a capture or adapter sequence at an end distal to the bead to which it is attached. The gRNA molecule (or precursor, portion, or derivative thereof) and the nucleic acid barcode molecule may be subjected to conditions sufficient to ligate the gRNA molecule (or precursor, portion, or derivative thereof) to the nucleic acid barcode molecule to provide a barcoded nucleic acid molecule comprising the sequences of the nucleic acid barcode molecule and sequences of the gRNA molecule (or precursor, portion, or derivative thereof). Additional processing may be performed within the partition (e.g., to further derivatize the barcoded nucleic acid molecule comprising a sequence of the gRNA molecule). Alternatively or in addition, the barcoded nucleic acid molecule (or a derivative thereof) may be recovered from the partition (e.g., by pooling contents of one or more different partitions, such as droplets of an emulsion). The recovered barcoded nucleic acid molecule (or derivative thereof) may be subjected to additional processing to, e.g., prepare the barcoded nucleic acid molecule for sequencing. For example, the recovered barcoded nucleic acid molecule (or derivative thereof) may be functionalized with one or more flow cell adapter sequences, sequencing primers, or other sequences (e.g., as described herein).

In another example, a gRNA molecule (e.g., a gRNA molecule produced within a cell), or a precursor, portion, or a derivative thereof, any of which may be associated with the gRNA or a gRNA-identifying barcode, may be subjected to processing. The gRNA molecule (or precursor, portion, or derivative thereof) within a biological particle (e.g., cell, cell bead, or cell nucleus) may be processed to provide a barcoded nucleic acid molecule. The biological particle may be disposed within a partition (e.g., a droplet or well) along with a bead comprising one or more nucleic acid barcode molecules (e.g., as described herein). A nucleic acid barcode molecule attached to the bead may be releasably attached from the bead and may be released from the bead (e.g., upon application of a stimulus and/or partial or complete degradation or dissolution of the bead within the partition). The nucleic acid barcode molecule may comprise a barcode sequence, a unique molecular identifier sequence, a flow cell adapter sequence or portion thereof, a capture or adapter sequence, or any other useful sequence, including a spacer sequence, arranged in any useful order. For example, the nucleic acid barcode molecule may comprise a capture or adapter sequence at an end distal to the bead to which it is attached. The partition may also comprise a primer molecule comprising a sequence complementary to a sequence of the gRNA molecule, such as a sequence complementary to a crRNA sequence or a tracrRNA sequence of the gRNA molecule. In some instances, the gRNA molecule (or precursor, portion, or derivative thereof) comprises a primer sequence that is complementary to the sequence of the primer molecule. The primer molecule and the gRNA molecule (or precursor, portion, or derivative thereof) may be subjected to conditions sufficient for the primer molecule to hybridize to the complementary sequence of the gRNA molecule (or precursor, portion, or derivative thereof). A primer extension reaction may be performed to provide an extended primer molecule comprising the sequence of the primer molecule complementary to the sequence of the gRNA molecule (or precursor, portion, or derivative thereof) and an additional sequence complementary to an additional sequence of the gRNA molecule (or precursor, portion, or derivative thereof). The extended primer molecule may additionally comprise an overhang sequence extending in a direction opposite the additional sequence complementary to the additional sequence of the gRNA molecule (or precursor, portion, or derivative thereof). The extended primer molecule and the nucleic acid barcode molecule may be subjected to conditions sufficient to ligate the extended primer molecule to the nucleic acid barcode molecule to provide a barcoded nucleic acid molecule comprising the sequences of the nucleic acid barcode molecule and sequences of the extended primer molecule, which comprises sequences complementary to the gRNA molecule (or precursor, portion, or derivative thereof). Additional processing may be performed within the partition (e.g., to further derivatize the barcoded nucleic acid molecule comprising a sequence of the gRNA molecule (or precursor, portion, or derivative thereof)). Alternatively or in addition, the barcoded nucleic acid molecule (or a derivative thereof) may be recovered from the partition (e.g., by pooling contents of one or more different partitions, such as droplets of an emulsion). The recovered barcoded nucleic acid molecule (or derivative thereof) may be subjected to additional processing to, e.g., prepare the barcoded nucleic acid molecule for sequencing. For example, the recovered barcoded nucleic acid molecule (or derivative thereof) may be functionalized with one or more flow cell adapter sequences, sequencing primers, or other sequences (e.g., as described herein).

In another example, a gRNA molecule (e.g., a gRNA molecule produced within a cell), or a precursor, portion, or a derivative thereof, any of which may be associated with the gRNA or a gRNA-identifying barcode, may be subjected to processing. The gRNA molecule (or precursor, portion, or derivative thereof) within a biological particle (e.g., cell, cell bead, or cell nucleus) may be processed to provide a barcoded nucleic acid molecule. The biological particle may be disposed within a partition (e.g., a droplet or well) along with a bead comprising one or more nucleic acid barcode molecules (e.g., as described herein). A nucleic acid barcode molecule attached to the bead may be releasably attached from the bead and may be released from the bead (e.g., upon application of a stimulus and/or partial or complete degradation or dissolution of the bead within the partition). The nucleic acid barcode molecule may comprise a barcode sequence, a unique molecular identifier sequence, a flow cell adapter sequence or portion thereof, a capture or adapter sequence, or any other useful sequence, including a spacer sequence, arranged in any useful order. For example, the nucleic acid barcode molecule may comprise a capture or adapter sequence at an end distal to the bead to which it is attached. The partition may also comprise a primer molecule comprising a sequence complementary to a sequence of the gRNA molecule, such as a sequence complementary to a crRNA sequence or a tracrRNA sequence of the gRNA molecule. The primer molecule and the gRNA molecule may be subjected to conditions sufficient for the primer molecule to hybridize to the complementary sequence of the gRNA molecule. A primer extension reaction may be performed to provide an extended primer molecule comprising the sequence of the primer molecule complementary to the sequence of the gRNA molecule and an additional sequence complementary to an additional sequence of the gRNA molecule. The extended primer molecule may additionally comprise an overhang sequence extending in a direction opposite the additional sequence complementary to the additional sequence of the gRNA molecule. The overhang sequence may be complementary to a sequence (e.g., a terminal sequence) of the nucleic acid barcode molecule. The extended primer molecule and the nucleic acid barcode molecule may be subjected to conditions sufficient to hybridize the overhang sequence of the extended primer molecule to the complementary sequence of the nucleic acid barcode molecule. A primer extension reaction may be performed to provide an extended barcoded product that is a barcoded nucleic acid molecule comprising sequences of the nucleic acid barcode molecule as well as sequences of the gRNA molecule or a derivative thereof. Additional processing may be performed within the partition (e.g., to further derivatize the barcoded nucleic acid molecule comprising a sequence of the gRNA molecule). Alternatively or in addition, the barcoded nucleic acid molecule (or a derivative thereof) may be recovered from the partition (e.g., by pooling contents of one or more different partitions, such as droplets of an emulsion). The recovered barcoded nucleic acid molecule (or derivative thereof) may be subjected to additional processing to, e.g., prepare the barcoded nucleic acid molecule for sequencing. For example, the recovered barcoded nucleic acid molecule (or derivative thereof) may be functionalized with one or more flow cell adapter sequences, sequencing primers, or other sequences (e.g., as described herein).

In another example, a gRNA molecule (e.g., a gRNA molecule produced within a cell, or a precursor, portion, or a derivative thereof, any of which may be associated with the gRNA or a gRNA-identifying barcode) within a biological particle (e.g., cell, cell bead, or cell nucleus) may be processed to provide a barcoded nucleic acid molecule. The biological particle may be disposed within a partition (e.g., a droplet or well) along with a bead comprising one or more nucleic acid barcode molecules (e.g., as described herein). A nucleic acid barcode molecule attached to the bead may be releasably attached from the bead and may be released from the bead (e.g., upon application of a stimulus and/or partial or complete degradation or dissolution of the bead within the partition). The nucleic acid barcode molecule may comprise a barcode sequence, a unique molecular identifier sequence, a flow cell adapter sequence or portion thereof, a capture or adapter sequence, or any other useful sequence, including a spacer sequence, arranged in any useful order. For example, the nucleic acid barcode molecule may comprise a capture or adapter sequence at an end distal to the bead to which it is attached. The partition may also comprise a primer molecule comprising a sequence complementary to a sequence of the gRNA molecule (or precursor or derivative thereof), such as a sequence complementary to a crRNA sequence or a tracrRNA sequence of the gRNA molecule (or precursor or derivative thereof). In some instances, the gRNA molecule (or precursor or derivative thereof) comprises a primer sequence that is complementary to the sequence of the primer molecule. The primer molecule and the gRNA molecule (or precursor or derivative thereof) may be subjected to conditions sufficient for the primer molecule to hybridize to the complementary sequence of the gRNA molecule (or precursor or derivative thereof). A primer extension reaction may be performed to provide an extended primer molecule comprising the sequence of the primer molecule complementary to the sequence of the gRNA molecule (or precursor or derivative thereof) and an additional sequence complementary to an additional sequence of the gRNA molecule (or precursor or derivative thereof). The extended primer molecule may additionally comprise a sequence extending in a direction opposite the additional sequence complementary to the additional sequence of the gRNA molecule (or precursor or derivative thereof). A template switching process (e.g., as described herein) may be used to extend the gRNA molecule (or precursor or derivative thereof) to the end of the primer molecule. Alternatively, a polymerase may be used to extend the end of the gRNA molecule to the end of the primer molecule. The sequence of the extended primer molecule extending in a direction opposite the additional sequence complementary to the additional sequence of the gRNA molecule may be complementary to a sequence (e.g., a terminal sequence) of the nucleic acid barcode molecule. The extended primer molecule and the nucleic acid barcode molecule may be subjected to conditions sufficient to hybridize the extending sequence of the extended primer molecule to the complementary sequence of the nucleic acid barcode molecule. A primer extension reaction may be performed to provide an extended barcoded product that is a barcoded nucleic acid molecule comprising sequences of the nucleic acid barcode molecule as well as sequences of the gRNA molecule or a derivative thereof. Additional processing may be performed within the partition (e.g., to further derivatize the barcoded nucleic acid molecule comprising a sequence of the gRNA molecule). Alternatively or in addition, the barcoded nucleic acid molecule (or a derivative thereof) may be recovered from the partition (e.g., by pooling contents of one or more different partitions, such as droplets of an emulsion). The recovered barcoded nucleic acid molecule (or derivative thereof) may be subjected to additional processing to, e.g., prepare the barcoded nucleic acid molecule for sequencing. For example, the recovered barcoded nucleic acid molecule (or derivative thereof) may be functionalized with one or more flow cell adapter sequences, sequencing primers, or other sequences (e.g., as described herein).

In another example, a gRNA molecule (e.g., a gRNA molecule produced within a cell), or a precursor, portion, or a derivative thereof, any of which may be associated with the gRNA or a gRNA-identifying barcode, may be subjected to processing. The gRNA (or precursor, portion, or derivative thereof) within a biological particle (e.g., cell, cell bead, or cell nucleus) may be processed to provide a barcoded nucleic acid molecule. The biological particle may be disposed within a partition (e.g., a droplet or well) along with a bead comprising one or more nucleic acid barcode molecules (e.g., as described herein). A nucleic acid barcode molecule attached to the bead may be releasably attached from the bead and may be released from the bead (e.g., upon application of a stimulus and/or partial or complete degradation or dissolution of the bead within the partition). The nucleic acid barcode molecule may comprise a barcode sequence, a unique molecular identifier sequence, a flow cell adapter sequence or portion thereof, a capture or adapter sequence, or any other useful sequence, including a spacer sequence, arranged in any useful order. For example, the nucleic acid barcode molecule may comprise a capture or adapter sequence at an end distal to the bead to which it is attached. The partition may also comprise a first primer molecule comprising a first sequence complementary to a first sequence of the gRNA molecule, such as a sequence complementary to a crRNA sequence or a tracrRNA sequence of the gRNA molecule, and a second primer molecule comprising a second sequence complementary to a second sequence of the gRNA molecule, such as a sequence complementary to a crRNA sequence or a tracrRNA sequence of the gRNA molecule. The first sequence of the first primer molecule may be complementary to a crRNA sequence of the gRNA molecule (or derivative thereof) and the second sequence of the second primer molecule may be complementary to a tracrRNA sequence of the gRNA molecule (or derivative thereof). In some instances, the gRNA molecule (or precursor, portion, or derivative thereof) comprises (i) a first primer sequence that is complementary to the first sequence of the first primer molecule and (ii) a second primer sequence that is complementary to the second primer sequence of the second primer molecule. In such an example, the first primer sequence and the second primer sequence may be disposed at opposite ends of the gRNA sequence, the gRNA-identifying barcode sequence (if present), or both the gRNA sequence and gRNA-identifying sequence. A plurality of different first primer molecules comprising a plurality of different first nucleic acid sequences may be provided, where only a subset of the first primer molecules may comprise a sequence complementary to a crRNA sequence of a given gRNA molecule (or precursor, portion, or derivative thereof). In this manner, a biological particle within a partition may be screened to identify a gRNA molecule (or precursor, portion, or derivative thereof) included therein (e.g., to determine which gRNA molecule of a plurality of possible gRNA molecules was introduced to the biological particle). Parallel processing of chromatin (e.g., as described herein) may establish a link between a perturbation introduced to a cell and genetic reprogramming. RNA-seq may also be performed in parallel to examine differential expression resulting from such genetic reprogramming.

The first and second primer molecules and the gRNA molecule (or a precursor, portion, or a derivative thereof, any of which may be associated with the gRNA or a gRNA-identifying barcode) may be subjected to conditions sufficient for the first sequence of the first primer molecule to hybridize to the first sequence of the gRNA molecule (or precursor, portion, or derivative thereof) and for the second sequence of the second primer molecule to hybridize to the second sequence of the gRNA molecule (or precursor, portion, or derivative thereof). The first sequence may comprise a first primer sequence and the second sequence may comprise the second primer sequence, which, as described herein, may be disposed at opposite ends of the gRNA sequence, the gRNA-identifying barcode sequence (if present), or both the gRNA sequence and the gRNA-identifying sequence. The first and/or second primer molecules may comprise any additional functional sequences, e.g., a flow cell adapter sequence or portion thereof, a capture or adapter sequence, a barcode sequence, a unique molecular identifier sequence, or any other useful sequence, including a spacer sequence, arranged in any useful order. In some examples, the primer molecule (e.g., first and/or second primer molecule) comprises a sequencing primer sequence, a sequencing primer binding sequence, a partial sequencing primer sequence, or a partial sequencing primer binding sequence. Primer extension reactions may be performed to provide a functionalized product comprising (i) the first sequence of the first primer molecule complementary to the first sequence of the gRNA molecule (or precursor, portion, or derivative thereof), an additional sequence complementary to an additional sequence of the gRNA molecule (or precursor, portion, or derivative thereof), and the second sequence of the second primer molecule complementary to the second sequence of the gRNA molecule (or precursor, portion, or derivative thereof) or a complement thereof in a first strand and (i) the first sequence of the first primer molecule complementary to the first sequence of the gRNA molecule (or precursor, portion, or derivative thereof) or complement thereof, an additional sequence of the gRNA molecule (or precursor, portion, or derivative thereof), and the second sequence of the second primer molecule complementary to the second sequence of the gRNA molecule (or precursor, portion, or derivative thereof) in a second strand. The functionalized product may additionally comprise one or more functional sequences at either end, such as one or more flow cell adapter sequences or sequencing primers. For example, the functionalized product may comprise a first sequencing primer (e.g., R1 sequence) or portion thereof, or complement thereof, at a first end and a second sequencing primer (e.g., R2 sequence) or portion thereof, or complement thereof, at a second end. In some cases, the functionalized product comprises a gRNA molecule (or a precursor, portion, or a derivative thereof, any of which may be associated with the gRNA or a gRNA-identifying barcode) flanked on each side with a functional sequence (e.g., a sequencing primer sequence, a sequencing primer binding sequence, a partial sequencing primer sequence, or a partial sequencing primer binding sequence). A sequence of the functionalized product (e.g., a sequencing primer sequence or portion thereof, or complement thereof) may be complementary to a sequence (e.g., a terminal sequence) of the nucleic acid barcode molecule, such as an adapter sequence of the nucleic acid barcode molecule. The functionalized product and the nucleic acid barcode molecule may be subjected to conditions sufficient to hybridize the complementary sequences. A primer extension reaction may be performed to provide an extended barcoded product that is a barcoded nucleic acid molecule comprising sequences of the nucleic acid barcode molecule as well as sequences of the gRNA molecule (or precursor, portion, or derivative thereof), or complements thereof, and sequences of each primer molecule, or complements thereof. Additional processing may be performed within the partition (e.g., to further derivatize the barcoded nucleic acid molecule comprising a sequence of the gRNA molecule). Alternatively or in addition, the barcoded nucleic acid molecule (or a derivative thereof) may be recovered from the partition (e.g., by pooling contents of one or more different partitions, such as droplets of an emulsion). The recovered barcoded nucleic acid molecule (or derivative thereof) may be subjected to additional processing to, e.g., prepare the barcoded nucleic acid molecule for sequencing. For example, the recovered barcoded nucleic acid molecule (or derivative thereof) may be functionalized with one or more flow cell adapter sequences, sequencing primers, or other sequences (e.g., as described herein).

Processing of a gRNA molecule (or a precursor, portion, or a derivative thereof, any of which may be associated with the gRNA or a gRNA-identifying barcode) may be performed in the absence of reverse transcription. For example, the gRNA molecule (or precursor, portion, or derivative thereof) may be directly processed (e.g., via a process involving ligation and/or functionalization with one or more primer molecules) without reverse transcription to generate complementary DNA (cDNA). Methods of processing gRNA molecules or derivatives thereof involving reverse transcription are described in, for example, International Patent publication No. PCT/US2018/066592, which is herein incorporated by reference in its entirety.

In some cases, parallel analysis of gRNA molecules (or precursors, portions, or derivatives thereof) may be performed. For example, a first process may be used to directly analyze a gRNA molecule (or precursor, portion, or derivative thereof) (e.g., as described herein) in the absence of reverse transcription, and a second process may be also be used to analyze a gRNA molecule (or precursor, portion, or derivative thereof). The second process may comprise processing an mRNA transcript corresponding to the gRNA molecule or (or precursor, portion, or derivative thereof), which mRNA transcript may be subjected to processing including reverse transcription to provide cDNA. Similarly, parallel analysis of gRNA molecules (or precursors, portions, or derivatives thereof) and other RNA molecules of a biological particle may be performed. Analysis of other RNA molecules (e.g., mRNA molecules) of a biological particle may comprise reverse transcription of an RNA molecule to generate a complementary cDNA strand, which cDNA strand may be barcoded. In some cases, template switching can be used to increase the length of a cDNA (e.g., via incorporation of one or more sequences, such as one or more barcode or unique molecular identifier sequences). In one example of template switching, cDNA can be generated from reverse transcription of a template (e.g., an mRNA molecule) where a reverse transcriptase with terminal transferase activity can add additional nucleotides, e.g., polyC, to the cDNA that are not encoded by the template, such, as at an end of the cDNA. Template switch oligonucleotides (e g., switch oligos) can include sequences complementary to the additional nucleotides, e.g. polyG (such as poly-riboG). The additional nucleotides (e.g., polyC) on the cDNA can hybridize to the sequences complementary to the additional nucleotides (e.g., polyG) on the template switch oligonucleotide, whereby the template switch oligonucleotide can be used by the reverse transcriptase as template to further extend the cDNA. Template switch oligonucleotides may comprise deoxyribonucleic acids, ribonucleic acids, modified nucleic acids including locked nucleic acids (LNA), or any combination thereof. A template switch oligonucleotide may comprise one or more sequences including, for example, one or more sequences selected from the group consisting of a sequencing primer, a barcode sequence, a unique molecular identifier sequence, and a homopolymer sequence (e.g., a polyG sequence), or a complement of any of the preceding sequence.

In some cases, the length of a template switch oligonucleotide may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 nucleotides or longer.

In some cases, an adapter and/or barcode sequence may be added to an RNA molecule via a method other than template switching. For example, one or more sequences may be ligated to an end of an RNA molecule. Similarly, one or more sequences may be ligated to an end of a cDNA molecule generated via reverse transcription of an RNA molecule.

In an example, a biological particle (e.g., cell, cell bead, or cell nucleus) comprising chromatin and one or more gRNA molecules (or a precursors, portions, or a derivatives thereof, any of which may be associated with the gRNA or a gRNA-identifying barcode e.g., as described herein) is provided. The chromatin in the biological particle (e.g., cell, cell bead, or cell nucleus) may be processed to provide a first template nucleic acid fragment derived from the chromatin (e.g., a tagmented fragment, as described herein). The chromatin may be processed in bulk solution. A gRNA molecule (or precursor, portion, or derivative thereof) may be optionally be processed to provide a second template nucleic acid fragment derived from the gRNA molecule (e.g., as described herein). The gRNA molecule (or precursor, portion, or derivative thereof) may be processed within a partition. The configuration of the first template nucleic acid fragment (e.g., chromatin fragment) may be at least partially dependent on the structure of the transposase-nucleic acid complex used to generate the first template nucleic acid fragment. For example, a transposase-nucleic acid complex such as that shown in FIG. 9 may be used to prepare the first template nucleic acid fragment. The first template nucleic acid fragment may be at least partially double-stranded. The first template nucleic acid fragment may comprise a double-stranded region comprising sequences of chromatin of the cell, cell bead, or cell nucleus. A first end of a first strand of the double-stranded region may be linked to a first transposon end sequence, which first transposon end sequence may be linked to a first sequencing primer or portion thereof. A first end of the second strand of the double-stranded region, which end is opposite the first end of the first strand, may be linked to a second transposon end sequence, which second transposon end sequence may be linked to a second sequencing primer or portion thereof. The second transposon end sequence may be the same as or different from the first transposon end sequence. The first sequencing primer or portion thereof may be the same as or different from the second sequencing primer or portion thereof. In some cases, the first sequencing primer or portion thereof may be an R1 sequence or portion thereof, and the second sequencing primer or portion thereof may be an R2 sequence or portion thereof. The first transposon end sequence may be hybridized to a first complementary sequence, which first complementary sequence may not be linked to a second end of the second strand of the double-stranded region of the first template nucleic acid fragment. Similarly, the second transposon end sequence may be hybridized to a second complementary sequence, which second complementary sequence may not be linked to a second end of the first strand of the double-stranded region of the first template nucleic acid fragment. In other words, the first template nucleic acid fragment may comprise one or more gaps. In some cases, the one or more gaps may be approximately 9 bp in length each. The second template nucleic acid fragment (e.g., an additional template nucleic acid fragment) may comprise a sequence of a gRNA molecule (or precursor, portion, or derivative thereof). Such a fragment may further comprise a sequence hybridized or ligated to a primer molecule (e.g., a capture nucleic acid molecule, as described herein). For example, the second template nucleic acid fragment may comprise a first sequence of a gRNA molecule (or precursor, portion, or derivative thereof) of the biological particle (e.g., cell, cell bead, or cell nucleus) and a second sequence of the gRNA molecule (or precursor, portion, or derivative thereof) hybridized or ligated to a sequence of a primer molecule. The primer molecule may also comprise an additional primer sequence. In some cases, two primer molecules (e.g., forward and reverse primers) may be used).

The biological particle (e.g., cell, cell bead, or cell nucleus) comprising the first template nucleic acid fragment (e.g., tagmented fragment) may be co-partitioned with one or more reagents into a partition of a plurality of partitions (e.g., as described herein). The partition may be, for example, a droplet or well. The partition may comprise one or more solid supports, e.g., beads (e.g., as described herein). A bead of the one or more supports (e.g., beads) may comprise a first plurality of nucleic acid barcode molecules. A nucleic acid barcode molecule of the first plurality of nucleic acid barcode molecules may comprise one or more of a flow cell adapter sequence (e.g., P5 sequence), a barcode sequence, a sequencing primer or portion thereof (e.g., R1 sequence or portion thereof, or a complement thereof), and a sequence configured to hybridize to a splint oligonucleotide as described elsewhere herein. The sequencing primer or portion thereof may be complementary to a sequence of the first template nucleic acid fragment. A support (e.g., bead) of the one or more supports (e.g., beads) may also comprise a second plurality of nucleic acid barcode molecules. A nucleic acid barcode molecule of the second plurality of nucleic acid barcode molecules may comprise one or more of a flow cell adapter sequence (e.g., P5 sequence), a barcode sequence, a sequencing primer or portion thereof (e.g., R1 sequence or portion thereof, or a complement thereof), and a sequence configured to hybridize to a splint oligonucleotide as described elsewhere herein. For example, a nucleic acid barcode molecule of the first plurality of nucleic acid barcode molecules may comprise one or more of a flow cell adapter sequence (e.g., P5 sequence), a barcode sequence, and a sequence configured to hybridize to a splint oligonucleotide as described elsewhere herein. A nucleic acid barcode molecule of a second plurality of nucleic acid barcode molecules attached to the bead may comprise a sequencing primer or portion thereof (e.g., R1 sequence or portion thereof, or a complement thereof), a barcode sequence, and a capture sequence (e.g., a poly T sequence) configured to hybridize to a nucleic acid molecule (e.g., gRNA molecule (or precursor, portion, or derivative thereof)), or to a sequence of a primer molecule coupled (e.g., hybridized or ligated) to a nucleic acid molecule (e.g., gRNA molecule (or precursor, portion, or derivative thereof)). In some cases, the first plurality of nucleic acid barcode molecules and the second plurality of nucleic acid barcode molecules may be same, such that a first nucleic acid barcode molecule of the first plurality of nucleic acid barcode molecules and a second nucleic acid barcode molecule of the second plurality of nucleic acid barcode molecules comprise the same sequence. In some cases, a first nucleic acid barcode molecule of the first plurality of nucleic acid barcode molecules and a second nucleic acid barcode molecule of the second plurality of nucleic acid barcode molecules comprise the same sequence excepting a unique molecular identifier sequence that may vary across the population of nucleic acid barcode molecules. In some cases, a solid support, e.g., a bead, may comprise a plurality of nucleic acid barcode molecules with each of nucleic acid barcode molecule comprising the same sequence such that both template nucleic acid fragments deriving from DNA molecules (e.g., tagmented fragments of open chromatin) and gRNA molecules (or a precursors, portions, or a derivatives thereof, any of which may be associated with the gRNA or a gRNA-identifying barcode) may be captured on the same support (e.g., bead)

Within the partition, th gRNA molecule (or precursor, portion, or derivative thereof) may be processed to provide the second template nucleic acid fragment (e.g., as described herein).

Within the partition, the biological particle (e.g., cell, cell bead, or cell nucleus) may be lysed or permeabilized to provide access to the first and/or second template nucleic acid fragments therein (e.g., as described herein). The second template nucleic acid fragment may be generated after the biological particle (e.g., cell, cell bead, or cell nucleus) is lysed or permeabilized.

The first and second template nucleic acid fragments may undergo processing within the partition. Within the partition, the gaps in the first template nucleic acid molecule may be filled via a gap filling extension process (e.g., using a DNA polymerase or reverse transcriptase). The resultant double-stranded nucleic acid molecule may be denatured to provide a single strand comprising a chromatin sequence flanked by transposon end sequences and/or sequences complementary to transposon end sequences. Each transposon end sequence and/or sequence complementary to transposon end sequence may be linked to a sequencing primer or portion thereof, or a complement thereof (e.g., an R1 or R2 sequence or a portion thereof, or a complement thereof). A nucleic acid barcode molecule of the first plurality of nucleic acid barcode molecules may hybridize to a sequencing primer or portion thereof, or a complement thereof, of the single strand. A primer extension reaction may then be used to generate a complement of the single strand (e.g., using a DNA polymerase or reverse transcriptase). Such a process may amount to a linear amplification process. This process incorporates the barcode sequence of the nucleic acid barcode molecule of the first plurality of nucleic acid barcode molecules, or a complement thereof. The resultant double-stranded molecule may be denatured to provide a single strand comprising the flow cell adapter sequence, or complement thereof, of the nucleic acid barcode molecule of the first plurality of nucleic acid barcode molecules; barcode sequence, or complement thereof, of the nucleic acid barcode molecule of the first plurality of nucleic acid barcode molecules; sequencing primer or portion thereof, or complement thereof, of the nucleic acid barcode molecule of the first plurality of nucleic acid barcode molecules; transposon end sequences, and/or complements thereof; second sequencing primer or portion thereof, or complement thereof. An additional amplification process may or may not be performed within a partition. For example, exponential amplification may or may not be performed within a partition.

Within the partition, th gRNA molecule (or precursor, portion, or derivative thereof) (e.g., second template nucleic acid fragment) may undergo processing to provide a barcoded nucleic acid molecule. For example, a gRNA molecule (or precursor, portion, or derivative thereof) may undergo a ligation process to couple the gRNA molecule (or precursor, portion, or derivative thereof) to a nucleic acid barcode molecule of the second plurality of nucleic acid barcode molecules, thereby providing a barcoded nucleic acid molecule comprising sequences of the nucleic acid barcode molecule as well as sequences of the gRNA molecule (or precursor, portion, or derivative thereof). In another example, a sequence of a gRNA molecule (or precursor, portion, or derivative thereof) may hybridize to a sequence of a primer molecule, after which a primer extension reaction may be performed to generate an extended primer product comprising sequences of the primer molecule and sequences complementary to the gRNA molecule (or precursor, portion, or derivative thereof). The extended primer product may be ligated to a nucleic acid barcode molecule of the second plurality of nucleic acid barcode molecules to provide a barcoded nucleic acid molecule. Alternatively, a sequence of the extended primer product, such as an overhang sequence, may hybridize to a sequence of the nucleic acid barcode molecule to provide the barcoded nucleic acid molecule. In another example, two primer molecules may be used, one of which may comprise a sequence configured to hybridize to a first sequence of the gRNA molecule (or precursor, portion, or derivative thereof) and the other of which may comprise a sequence configured to hybridize to a second sequence of the gRNA molecule (or precursor, portion, or derivative thereof). Following hybridization, primer extension reactions may be performed to provide a functionalized product comprising sequences of the gRNA molecule (or precursor, portion, or derivative thereof), and complements thereof, as well as sequences of the primer molecules, and complements thereof. A sequence of the functionalized product may hybridize or ligate to a sequence of a nucleic acid barcode molecule of the second plurality of nucleic acid barcode molecules, thereby providing a barcoded nucleic acid molecule. Additional processing of a barcoded nucleic acid molecule comprising sequences of a gRNA molecule (or precursor, portion, or derivative thereof), or complements thereof, may optionally be performed in the partition. For example, one or more functional sequences may be introduced. An additional amplification process may or may not be performed within a partition. For example, exponential amplification may or may not be performed within a partition.

If processing of other RNA molecules is performed in parallel, such a process may comprise reverse transcribing (e.g., using a reverse transcriptase) the RNA molecule or a derivative thereof (e.g., fragment derived therefrom) to provide a cDNA strand within a partition.

The reverse transcription process may append a sequence to an end of a strand of the resultant double-stranded nucleic acid molecule comprising the RNA strand and the cDNA strand, such as a polyC sequence. A template switching oligonucleotide may comprise a sequence (e.g., a polyG sequence) that may hybridize to at least a portion of the double-stranded nucleic acid molecule (e.g., to the appended polyC sequence) and be used to further extend the strand of the double-stranded nucleic acid molecule to provide an extended double-stranded nucleic acid molecule. Such a sequence may comprise ribobases. The template switching oligonucleotide may comprise a UMI sequence, or complement thereof, and a sequencing primer or portion thereof, or complement thereof. The extended double-stranded nucleic acid molecule comprising the template switching oligonucleotide and a complement thereof, and the prior double-stranded nucleic acid molecule may be denatured to provide a single strand comprising a sequencing primer or portion thereof, or complement thereof, of the nucleic acid barcode molecule of the second plurality of nucleic acid barcode molecules; the UMI sequence, or complement thereof; the poly(C) or poly(G) sequence; the sequence corresponding to the RNA molecule of the cell, cell bead, or cell nucleus, or complement thereof; and sequences of the capture nucleic acid molecule, or complements thereof. A nucleic acid barcode molecule of the second plurality of nucleic acid barcode molecules may hybridize to a sequencing primer or portion thereof, or a complement thereof, of the single strand. A primer extension reaction may then be used to generate a complement of the single strand (e.g., using a DNA polymerase). Such a process may amount to a linear amplification process. This process incorporates the barcode sequence of the nucleic acid barcode molecule of the second plurality of nucleic acid barcode molecules, or a complement thereof. The resultant double-stranded molecule may be denatured to provide a single strand comprising a flow cell adapter sequence, or complement thereof, of the nucleic acid barcode molecule of the second plurality of nucleic acid barcode molecules; a barcode sequence, or complement thereof, of the nucleic acid barcode molecule of the second plurality of nucleic acid barcode molecules; a sequencing primer or portion thereof, or complement thereof, of the nucleic acid barcode molecule of the second plurality of nucleic acid barcode molecules; the UMI sequence, or complement thereof; the poly(C) or poly(G) sequence; the sequence corresponding to the RNA molecule of the cell, cell bead, or cell nucleus, or complement thereof; and sequences of the capture nucleic acid molecule, or complements thereof. An additional amplification process may or may not be performed within a partition. For example, exponential amplification may or may not be performed within a partition. Additional examples of RNA processing performed in parallel with chromatin processing (e.g., parallel ATACseq and RNAseq) are described in PCT/US2020/018010, which is herein incorporated by reference in its entirety.

The barcoded products (e.g., linear amplification products corresponding to the chromatin and the gRNA molecule (or precursor, portion, or derivative thereof) of the biological particle (e.g., cell, cell bead, or cell nucleus) included within the partition of the plurality of partitions may be recovered from the partition. For example, the contents of the plurality of partitions may be pooled to provide the barcoded products (e.g., linear amplification products) in a bulk solution. The linear amplification product corresponding to the open chromatin may then be subjected to conditions sufficient to undergo one or more nucleic acid amplification reactions (e.g., PCR) to generate one or more amplification products corresponding to the open chromatin. A nucleic acid amplification process may incorporate one or more additional sequences, such as one or more additional flow cell adapter sequences. The linear amplification product corresponding to the gRNA molecule (or precursor, portion, or derivative thereof) may be subjected to fragmentation, end repair, and dA tailing processes. An additional primer sequence (e.g., a sequencing primer or portion thereof, such as an R2 sequence) may then be ligated to the resultant molecule. A nucleic acid amplification reaction (e.g., PCR) may then be performed to generate one or more amplification products corresponding to the gRNA molecule (or precursor, portion, or derivative thereof). A nucleic acid amplification process may incorporate one or more additional sequences, such as one or more additional flow cell adapter sequences.

In an RNA workflow, in-partition template switching may attach a sequencing primer (e.g., a TruSeq R1 or R2 sequence) to the 3′ or 5′ end of an RNA transcript. A bead (e.g., gel bead) carrying the sequencing primer, or portion thereof (e.g., partial TruSeq R1 or R2 sequence) may be also used for priming in a DNA (e.g., chromatin) workflow. This may allow for differential amplification of DNA (e.g., ATAC) and RNA libraries after removing materials from partitions (e.g., breaking emulsions) and sample splitting. Another advantage of this method is that the same enzyme (e.g. DNA polymerase or reverse transcriptase) may be used to barcode nucleic acid fragments derived from both DNA (e.g., chromatin) and RNA. In some embodiments, in addition to tagmented fragments of open chromatin and nucleic acid molecules as perturbation agents or associated with such (e.g., gRNA molecules or precursors or derivatives thereof), additional nucleic acid molecules (e.g., mRNA) may be processed, see e.g., US. Pat. Pub. 20180340171 and US. Pat. Pub. 20200291454; each of which are herein incorporated by reference in their entireties.

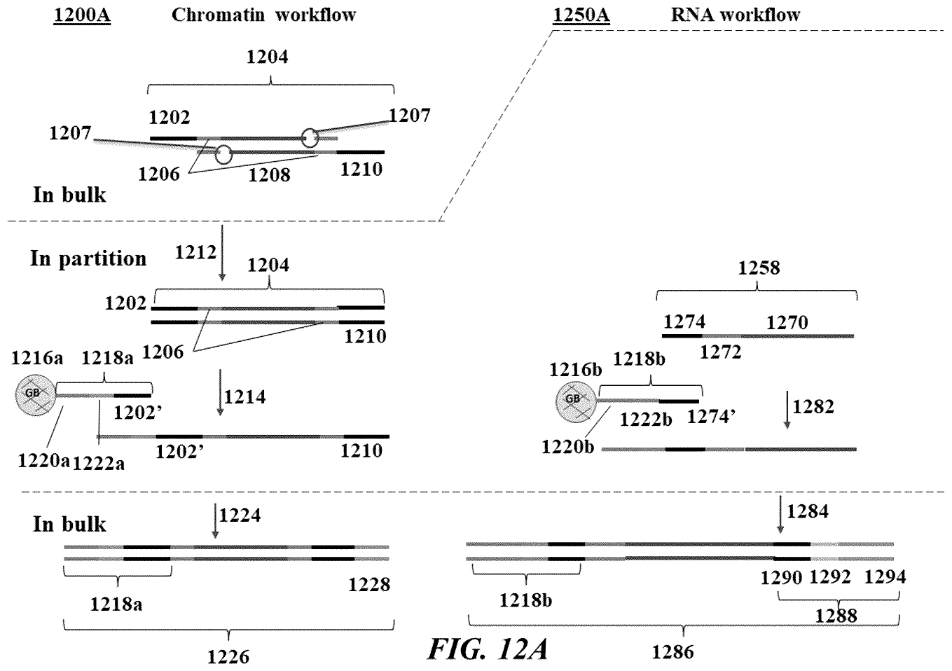

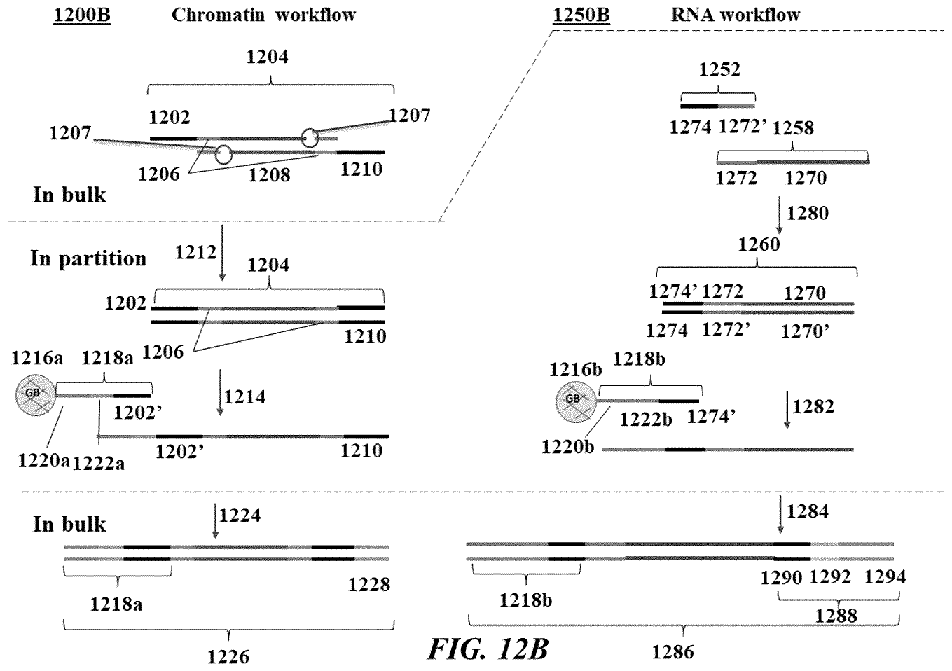

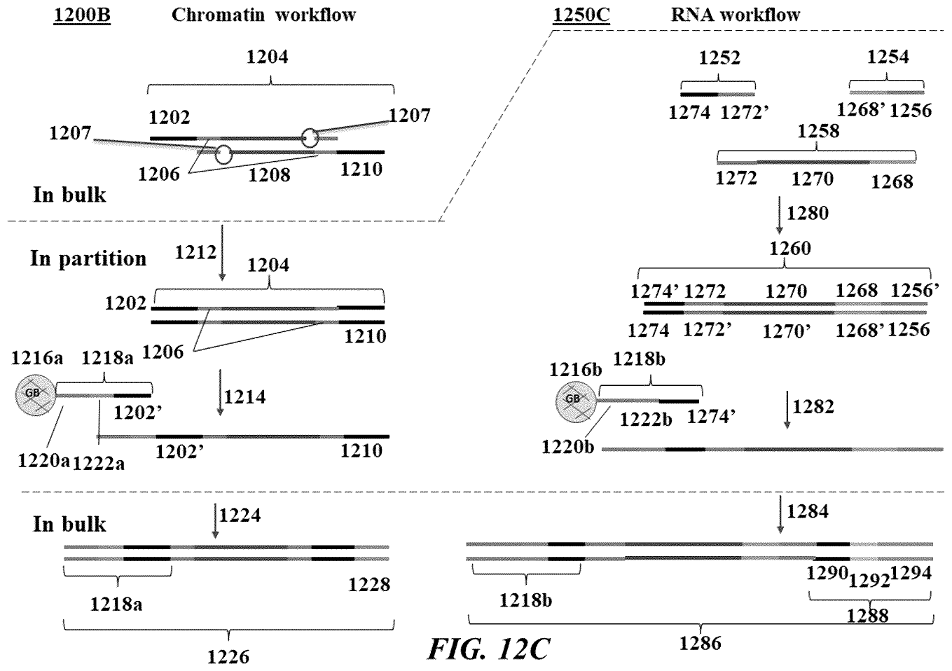

FIG. 12A shows an example schematic corresponding to the preceding example. Panel 1200A shows a workflow corresponding to processing of chromatin from a cell, cell bead, or cell nucleus, and panel 1250A shows a workflow corresponding to processing of a gRNA molecule (or precursor, portion, or derivative thereof) from the biological particle (e.g., cell, cell bead, or cell nucleus). In the figure, two distinct beads (e.g., gel beads) are shown. However, the same bead (e.g., a single gel bead that may be a multifunctional bead) may be used in each workflow. In some embodiments, tagmented fragments, gRNA molecules (or a precursor, portion, or a derivative thereof, any of which may be associated with the gRNA or a gRNA-identifying barcode) and/or mRNA molecules from the cell, cell bead, or cell nucleus may be processed as provided herein.

As shown in panel 1200A, in bulk solution, chromatin included within a cell, cell bead, or cell nucleus is processed (e.g., as described herein) to provide a template nucleic acid fragment (e.g., tagmented fragment) 1204 comprising insert sequence 1208 (e.g., region of open chromatin) and a complement thereof, transposon end sequences 1206 and complements thereof, sequencing primer or portion thereof 1202 (e.g., an R1 sequence), sequencing primer or portion thereof 1210 (e.g., an R2 sequence), and gaps 1207. Template nucleic acid fragment 1204 may then be partitioned within a partition (e.g., a droplet or well, as described herein). Within the partition, the cell, cell bead, or cell nucleus comprising template nucleic acid fragment 1204 may be lysed, permeabilized, or otherwise processed to provide access to template nucleic acid fragment 1204 (and one or more RNA molecules) therein. Gaps 1207 may be filled 1212 via a gap filling extension process (e.g., using a DNA polymerase). The partition may include a bead (e.g., gel bead) 1216a coupled to a nucleic acid barcode molecule 1218a. Nucleic acid barcode molecule 1218a may comprise a flow cell adapter sequence 1220a (e.g., a P5 sequence), a barcode sequence 1222a, and a sequencing primer or portion thereof or complement thereof 1202′. Sequence 1202′ may hybridize to sequence 1202 of template nucleic acid fragment 1204, or its complement, and undergo primer extension 1214 to yield a strand comprising sequences 1220a, 1222a, 1202′, 1210, and insert sequence 1208 or a complement thereof. The contents of the partition may then be recovered in bulk solution (e.g., a droplet may be broken) to provide the strand in bulk solution. This strand may undergo amplification (e.g., PCR) 1224 to provide a double-stranded amplification product 1226 that includes sequences of the nucleic acid barcode molecule 1218a, the original chromatin molecule, and, optionally, an additional sequence 1228 that may be a flow cell adapter sequence (e.g., a P7 sequence).

In parallel to the chromatin workflow of panel 1200A, a gRNA molecule or precursor, portion, or derivative thereof of (e.g., directly provided to, or produced therein upon provision of a precursor) the same biological particle may be processed. As shown in panel 1250A, gRNA molecule (or precursor, portion, or derivative thereof) 1258 comprising RNA sequences 1270, 1272, and 1274 may be provided. It should be noted that this representation is in no way intended to be limiting and numerous additional sequences having different functionalities may be included, and/or one or more of these sequences may not be present, and/or these sequences may be arranged in any other useful order. RNA sequence 1270 may be a tracrRNA sequence while RNA sequence 1272 may be a crRNA sequence. Alternatively, RNA sequence 1270 may be a crRNA sequence while RNA sequence 1272 may be a tracrRNA sequence. These sequences may be separated by one or more additional sequences. gRNA molecule (or precursor, portion, or derivative thereof) 1258 may also comprise one or more additional sequences adjacent to sequence 1270. Sequence 1274 may be complementary to sequence 1274′ of nucleic acid barcode molecule 1218b coupled to bead 1216b. Sequence 1274 may optionally be introduced to gRNA molecule (or precursor, portion, or derivative thereof) via a functionalization process using, e.g., a primer molecule (e.g., as described herein). The partition may include a bead (e.g., gel bead) 1216b coupled to a nucleic acid barcode molecule 1218b. Nucleic acid barcode molecule 1218b may comprise a flow cell adapter sequence 1220b (e.g., a P5 sequence), a barcode sequence 1222b, and a sequencing primer or portion thereof or complement thereof 1274′. Bead 1216b may be the same as bead 1216a such that partition comprises a single bead (e.g., 1218a and 1218b are attached to a single bead). In such a case, nucleic acid barcode molecule 1218b and nucleic acid barcode molecule 1218a may have the same sequences. Sequence 1274′ may hybridize to sequence 1274 of gRNA molecule (or precursor, portion, or derivative thereof) 1258 and undergo primer extension 1282 to yield a strand comprising sequences 1220b, 1222b, 1274′, 1272 or a complement thereof, and 1270 or a complement thereof. The contents of the partition may then be recovered in bulk solution (e.g., a droplet may be broken) to provide the strand in bulk solution. This strand may undergo amplification (e.g., PCR) 1284 to provide a double-stranded amplification product 1286 that includes sequences of the nucleic acid barcode molecule 1218b, sequences corresponding to the original gRNA molecule (or precursor, portion, or derivative thereof), and, optionally, an additional sequence 1288 that may comprise a sequencing primer or portion thereof (e.g., an R2 sequence) 1290, a sample index sequence 1292, and a flow cell adapter sequence (e.g., a P7 sequence) 1294.

FIG. 12B shows another example schematic corresponding to the preceding example. Panel 1200B shows the same chromatin workflow shown in Panel 1200A of FIG. 12A. Panel 1250B shows another workflow corresponding to processing of a gRNA molecule (or precursor, portion, or derivative thereof) from the biological particle (e.g., cell, cell bead, or cell nucleus). In the figure, two distinct beads (e.g., gel beads) are shown. In some cases, the same bead may have a plurality of nucleic acid barcode molecules with the same sequences. However, the same bead (e.g., a single gel bead that may be a multifunctional bead) may be used in each workflow. In some embodiments, 1258 may be a gRNA molecule, a precursor (e.g., vector cassette comprising a gRNA sequence), a transcript generated from the precursor (e.g., vector) which comprises a sequence encoding a gRNA and/or a gRNA-identifying barcode, or a portion of the precursor (e.g., vector) or transcript. In some particular embodiments, processing of the nucleic acid molecule may include a reverse transcription step to provide cDNA.

As shown in panel 1250B, gRNA molecule (or precursor, portion, or derivative thereof) 1258 comprising RNA sequences 1270 and 1272 may be provided. It should be noted that this representation is in no way intended to be limiting and numerous additional sequences having different functionalities may be included, and/or one or more of these sequences may not be present, and/or these sequences may be arranged in any other useful order. RNA sequence 1270 may be a tracrRNA sequence while RNA sequence 1272 may be a crRNA sequence. Alternatively, RNA sequence 1270 may be a crRNA sequence while RNA sequence 1272 may be a tracrRNA sequence. Alternatively, sequence 1272 may be another sequence of gRNA molecule 1258 that is not a crRNA or tracrRNA sequence. In such an instance, sequence 1270 may include both a crRNA sequence and a tracrRNA sequence. These sequences may be separated by one or more additional sequences. gRNA molecule (or precursor, portion, or derivative thereof) 1258 may also comprise one or more additional sequences adjacent to sequence 1270 and/or sequence 1272. Primer molecule 1252 comprising sequences 1274 and 1272′ may also be included in the partition. Sequence 1272′ may be complementary to sequence 1272 (e.g., a crRNA sequence or tracrRNA sequence of the gRNA molecule, or another sequence of the gRNA molecule) and may hybridize to sequence 1272 and undergo primer extension in process 1280 to provide functionalized gRNA molecule 1260 comprising sequences 1274, 1272′, and 1270′ complementary to sequence 1270 in a first strand and sequences 1272 and 1270 in a second strand. This extended primer product may be further processed to include sequence 1274′ complementary to sequence 1274 in the second strand as shown. Sequence 1274 may be complementary to sequence 1274′ of nucleic acid barcode molecule 1218b coupled to bead 1216b. Sequence 1274 may optionally be introduced to gRNA molecule (or precursor, portion, or derivative thereof) via a functionalization process using, e.g., a primer molecule (e.g., as described herein). The partition may include a bead (e.g., gel bead) 1216b coupled to a nucleic acid barcode molecule 1218b. Nucleic acid barcode molecule 1218b may comprise a flow cell adapter sequence 1220b (e.g., a P5 sequence), a barcode sequence 1222b, and a sequencing primer or portion thereof or complement thereof 1274′. Bead 1216b may be the same as bead 1216a such that partition comprises a single bead (e.g., 1218a and 1218b are attached to a single bead). In such a case, nucleic acid barcode molecule 1218b and nucleic acid barcode molecule 1218a may have the same sequences. Sequence 1274′ may hybridize to sequence 1274 of gRNA molecule 1260 and undergo primer extension 1282 to yield a strand comprising sequences 1220b, 1222b, 1274′, 1272 or a complement thereof, and 1270 or a complement thereof, or complements of any such sequences. The contents of the partition may then be recovered in bulk solution (e.g., a droplet may be broken) to provide the strand in bulk solution. This strand may undergo amplification (e.g., PCR) 1284 to provide a double-stranded amplification product 1286 that includes sequences of the nucleic acid barcode molecule 1218b, sequences corresponding to the original gRNA molecule, and, optionally, an additional sequence 1288 that may comprise a sequencing primer or portion thereof (e.g., an R2 sequence) 1290, a sample index sequence 1292, and a flow cell adapter sequence (e.g., a P7 sequence) 1294.

FIG. 12C shows another example schematic corresponding to the preceding example. Panel 1200C shows the same chromatin workflow shown in Panel 1200A of FIG. 12A. Panel 1250C shows another workflow corresponding to processing of a gRNA molecule (or precursor, portion, or derivative thereof) from the biological particle (e.g., cell, cell bead, or cell nucleus). In the figure, two distinct beads (e.g., gel beads) are shown. However, the same bead (e.g., a single gel bead that may be a multifunctional bead) may be used in each workflow.

As shown in panel 1250C, gRNA molecule (or precursor, portion, or derivative thereof) 1258 comprising RNA sequences 1268, 1270, and 1272 may be provided. It should be noted that this representation is in no way intended to be limiting and numerous additional sequences having different functionalities may be included, and/or one or more of these sequences may not be present, and/or these sequences may be arranged in any other useful order. One or more of RNA sequences 1268, 1270, and 1272 may comprise all or a portion of a crRNA sequence and/or a tracrRNA sequence. For example, RNA sequence 1270 may be a tracrRNA sequence while RNA sequence 1272 may be a crRNA sequence. Alternatively, RNA sequence 1270 may be a crRNA sequence while RNA sequence 1272 may be a tracrRNA sequence. Alternatively, RNA sequence 1268 may be a crRNA sequence while RNA sequence 1272 may be a tracrRNA sequence. Alternatively, RNA sequence 1272 may be a crRNA sequence while RNA sequence 1268 may be a tracrRNA sequence. Alternatively, RNA sequence 1268 may be a crRNA sequence while RNA sequence 1270 may be a tracrRNA sequence. Alternatively, RNA sequence 1270 may be a crRNA sequence while RNA sequence 1268 may be a tracrRNA sequence. Alternatively, sequences 1272 and 1268 may not be crRNA or tracrRNA sequences. In such a case, sequence 1270 may comprise both a crRNA and a tracrRNA sequence.

RNA sequences 1268, 1270, and 1272 may sequences may be separated by one or more additional sequences. gRNA molecule (or precursor, portion, or derivative thereof) 1258 may also comprise one or more additional sequences adjacent to sequence 1268, 1270, and/or 1272. Primer molecules 1252 and 1254 may also be included in the partition. Primer molecule 1252 may comprise sequences 1274 and 1272′. Primer molecule 1254 may comprise sequences 1268′ and 1256. Sequence 1272′ may be complementary to sequence 1272 (e.g., a crRNA sequence or tracrRNA sequence of the gRNA molecule (or precursor, portion, or derivative thereof), or another sequence of the gRNA molecule) and may hybridize to sequence 1272 and undergo primer extension in process 1280. Sequence 1268′ may be complementary to sequence 1268 (e.g., a crRNA sequence or tracrRNA sequence of the gRNA molecule, or another sequence of the gRNA molecule) and may hybridize to sequence 1268 and undergo primer extension in process 1280. Process 1280 may provide functionalized gRNA molecule 1260 comprising sequences 1274, 1272′, 1270′ complementary to sequence 1270, 1268′, and 1256 in a first strand and sequences 1272, 1270, and 1268 in a second strand. This extended primer product may be further processed to include sequence 1274′ complementary to sequence 1274 and/or sequence 1256′ complementary to sequence 1256 in the second strand as shown. Sequence 1274 may be complementary to sequence 1274′ of nucleic acid barcode molecule 1218b coupled to bead 1216b. Sequence 1274 may optionally be introduced to gRNA molecule (or precursor, portion, or derivative thereof) via a functionalization process using, e.g., a primer molecule (e.g., as described herein). The partition may include a bead (e.g., gel bead) 1216b coupled to a nucleic acid barcode molecule 1218b. Nucleic acid barcode molecule 1218b may comprise a flow cell adapter sequence 1220b (e.g., a P5 sequence), a barcode sequence 1222b, and a sequencing primer or portion thereof or complement thereof 1274′. Bead 1216b may be the same as bead 1216a such that partition comprises a single bead (e.g., 1218a and 1218b are attached to a single bead). In such a case, nucleic acid barcode molecule 1218b and nucleic acid barcode molecule 1218a may have the same sequences. Sequence 1274′ may hybridize to sequence 1274 of gRNA molecule 1260 and undergo primer extension 1282 to yield a strand comprising sequences 1220b, 1222b, 1274′, 1272 or a complement thereof, 1270 or a complement thereof, 1268 or a complement thereof, 1256′, or complements of any such sequences. The contents of the partition may then be recovered in bulk solution (e.g., a droplet may be broken) to provide the strand in bulk solution. This strand may undergo amplification (e.g., PCR) 1284 to provide a double-stranded amplification product 1286 that includes sequences of the nucleic acid barcode molecule 1218b, sequences corresponding to the original gRNA molecule, and, optionally, an additional sequence 1288 that may comprise a sequencing primer or portion thereof (e.g., an R2 sequence) 1290, a sample index sequence 1292, and a flow cell adapter sequence (e.g., a P7 sequence) 1294.

In another example, a biological particle (e.g., cell, cell bead, or cell nucleus) comprising chromatin and one or more gRNA molecules (or a precursor, portion, or a derivative thereof, any of which may be associated with the gRNA or a gRNA-identifying barcode is provided. The chromatin in the biological particle (e.g., cell, cell bead, or cell nucleus) may be processed to provide a first template nucleic acid fragment derived from the chromatin (e.g., a tagmented fragment, as described herein). The chromatin may be processed in bulk solution. A gRNA molecule may optionally undergo processing and/or may be produced in the biological particle within a partition. The configuration of the first template nucleic acid fragment may be at least partially dependent on the structure of the transposase-nucleic acid complex used to generate the first template nucleic acid fragment. For example, a transposase-nucleic acid complex such as that shown in FIG. 9 may be used to prepare the first template nucleic acid fragment. The first template nucleic acid fragment may be at least partially double-stranded. The first template nucleic acid fragment may comprise a double-stranded region comprising sequences of chromatin of the cell, cell bead, or cell nucleus. A first end of a first strand of the double-stranded region may be linked to a first transposon end sequence, which first transposon end sequence may be linked to a first sequencing primer or portion thereof. A first end of the second strand of the double-stranded region, which end is opposite the first end of the first strand, may be linked to a second transposon end sequence, which second transposon end sequence may be linked to a second sequencing primer or portion thereof. The second transposon end sequence may be the same as or different from the first transposon end sequence. The first sequencing primer or portion thereof may be the same as or different from the second sequencing primer or portion thereof. In some cases, the first sequencing primer or portion thereof may be an R1 sequence or portion thereof, and the second sequencing primer or portion thereof may be an R2 sequence or portion thereof. The first transposon end sequence may be hybridized to a first complementary sequence, which first complementary sequence may not be linked to a second end of the second strand of the double-stranded region of the first template nucleic acid fragment. Similarly, the second transposon end sequence may be hybridized to a second complementary sequence, which second complementary sequence may not be linked to a second end of the first strand of the double-stranded region of the first template nucleic acid fragment. In other words, the first template nucleic acid fragment may comprise one or more gaps. In some cases, the one or more gaps may be approximately 9 bp in length each. For example, one or more gaps may be at least about 1, 2, 3, 4, 5, 6, 7, 8, 9, 10, or more bp in length. For example, one or more gaps may be at most about 10, 9, 8, 7, 6, 5, 4, 3, 2, or 1 bp in length. The second template nucleic acid fragment (e.g., an additional template nucleic acid fragment) may comprise a sequence of a gRNA molecule or derivative thereof. Such a fragment may further comprise a sequence hybridized or ligated to a primer molecule (e.g., a capture nucleic acid molecule, as described herein). For example, the second template nucleic acid fragment may comprise a first sequence of a gRNA molecule of the biological particle (e.g., cell, cell bead, or cell nucleus) and a second sequence of the gRNA molecule hybridized or ligated to a sequence of a primer molecule. The primer molecule may also comprise an additional primer sequence. In some cases, two primer molecules (e.g., forward and reverse primers) may be used).

The biological particle (e.g., cell, cell bead, or cell nucleus) comprising the first template nucleic acid fragment (e.g., tagmented fragment) may be co-partitioned with one or more reagents into a partition of a plurality of partitions (e.g., as described herein). The partition may be, for example, a droplet or well. The partition may comprise one or more beads (e.g., as described herein). A bead (e.g., gel bead) of the one or more beads may comprise a first plurality of nucleic acid barcode molecules. A nucleic acid barcode molecule of the first plurality of nucleic acid barcode molecules may comprise a flow cell adapter sequence (e.g., P5 sequence), a barcode sequence, and a sequencing primer or portion thereof (e.g., R1 sequence or portion thereof, or a complement thereof). The sequencing primer or portion thereof may be complementary to a sequence of the first template nucleic acid fragment. The flow cell adapter sequence and/or barcode sequence may be hybridized to their complementary sequences. A bead (e.g., gel bead) of the one or more beads may also comprise a second plurality of nucleic acid barcode molecules. A nucleic acid barcode molecule of the second plurality of nucleic acid barcode molecules may comprise a flow cell adapter sequence (e.g., P5 sequence), a barcode sequence, a sequencing primer or portion thereof (e.g., R1 sequence or portion thereof, or a complement thereof), a UMI sequence, and a capture sequence (e.g., a polyG sequence, a polydT sequence or target specific sequence). In some cases, the first plurality of nucleic acid barcode molecules and the second plurality of nucleic acid barcode molecules may be coupled to the same bead, and the partition may comprise a single bead.

Within the partition, the gRNA molecule may be processed to provide the second template nucleic acid fragment (e.g., as described herein). In some aspects, the processing of the second template nucleic acid fragment may include providing a plurality of primer molecules configured to amplify a sequence of the gRNA molecule (or a precursors, portions, or a derivatives thereof, any of which may be associated with the gRNA or a gRNA-identifying barcode). In some embodiments, the amplification step using the provided primer molecules may occur within the partition.

Within the partition, the biological particle (e.g., cell, cell bead, or cell nucleus) may be lysed or permeabilized to provide access to the first and/or second template nucleic acid fragments therein (e.g., as described herein). The second template nucleic acid fragment may be generated after the biological particle (e.g., cell, cell bead, or cell nucleus) is lysed or permeabilized.

The first and second template nucleic acid fragments may undergo processing within the partition. Within the partition, a sequencing primer or portion thereof of the first template nucleic acid fragment corresponding to the chromatin of the biological particle (e.g., cell, cell bead, or cell nucleus) may hybridize to a sequencing primer or portion thereof of the nucleic acid barcode molecule of the first plurality of nucleic acid barcode molecules. The sequencing primer or portion thereof of the nucleic acid barcode molecule may then be ligated (e.g., using a ligase) to a transposon end sequence of the first template nucleic acid fragment, or a complement thereof to provide a partially double-stranded nucleic acid molecule corresponding to the chromatin of the biological particle (e.g., cell, cell bead, or cell nucleus).

Within the partition, the gRNA molecule or derivative thereof (e.g., second template nucleic acid fragment) may undergo processing to provide a barcoded nucleic acid molecule (e.g., as described herein). For example, a gRNA molecule or derivative thereof may undergo a ligation process to couple the gRNA molecule or derivative thereof to a nucleic acid barcode molecule of the second plurality of nucleic acid barcode molecules, thereby providing a barcoded nucleic acid molecule comprising sequences of the nucleic acid barcode molecule as well as sequences of the gRNA molecule or derivative thereof. In another example, a sequence of a gRNA molecule or derivative thereof may hybridize to a sequence of a primer molecule, after which a primer extension reaction may be performed to generate an extended primer product comprising sequences of the primer molecule and sequences complementary to the gRNA molecule or derivative thereof. The extended primer product may be ligated to a nucleic acid barcode molecule of the second plurality of nucleic acid barcode molecules to provide a barcoded nucleic acid molecule. Alternatively, a sequence of the extended primer product, such as an overhang sequence, may hybridize to a sequence of the nucleic acid barcode molecule to provide the barcoded nucleic acid molecule. In another example, two primer molecules may be used, one of which may comprise a sequence configured to hybridize to a first sequence of the gRNA molecule or derivative thereof and the other of which may comprise a sequence configured to hybridize to a second sequence of the gRNA molecule or derivative thereof. Following hybridization, primer extension reactions may be performed to provide a functionalized product comprising sequences of the gRNA molecule or derivative thereof, and complements thereof, as well as sequences of the primer molecules, and complements thereof. A sequence of the functionalized product may hybridize or ligate to a sequence of a nucleic acid barcode molecule of the second plurality of nucleic acid barcode molecules, thereby providing a barcoded nucleic acid molecule. Additional processing of a barcoded nucleic acid molecule comprising sequences of a gRNA molecule or derivative thereof, or complements thereof, may optionally be performed in the partition. For example, one or more functional sequences may be introduced. An additional amplification process may or may not be performed within a partition. For example, exponential amplification may or may not be performed within a partition.

The partially double-stranded molecule corresponding to the chromatin of the biological particle (e.g., cell, cell bead, or cell nucleus) and the barcoded molecule corresponding to the gRNA molecule or derivative thereof of the biological particle (e.g., cell, cell bead, or cell nucleus) included within the partition (e.g., droplet or well) of the plurality of partitions may be recovered from the partition. For example, the contents of the plurality of partitions may be pooled to provide these products in a bulk solution.

Outside of the partition, the gaps in the partially double-stranded nucleic acid molecule corresponding to the chromatin may be filled using via a gap filling extension process (e.g., using a DNA polymerase or reverse transcriptase). The gap filling extension process may not include strand displacement. The resultant gap-filled double-stranded nucleic acid molecule may be denatured to provide a single strand, which single strand may be subjected to conditions sufficient to perform one or more nucleic acid amplification reactions (e.g., PCR) to generate amplification products corresponding to the chromatin of the cell, cell bead, or cell nucleus. A nucleic acid amplification process may incorporate one or more additional sequences, such as one or more additional flow cell adapter sequences.

Outside of the partition, the barcoded molecule corresponding to the gRNA molecule or derivative thereof may be subjected to fragmentation, end repair, a dA tailing process, tagmentation, or a combination thereof. An additional primer sequence (e.g., a sequencing primer or portion thereof, such as an R2 sequence) may be ligated to the resultant molecule. Alternatively or in addition, a nucleic acid amplification reaction (e.g., PCR) may be performed to generate one or more amplification products corresponding to the gRNA molecule or a derivative thereof. A nucleic acid amplification process may incorporate one or more additional sequences, such as one or more additional flow cell adapter sequences.

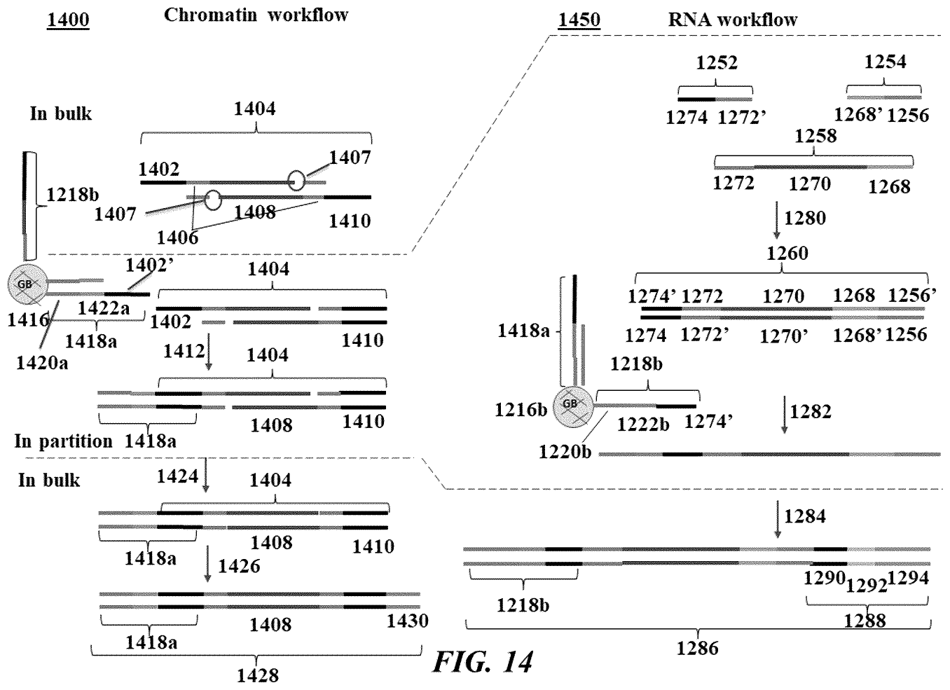

FIG. 14 shows an example schematic corresponding to the preceding example. Panel 1400 shows a workflow corresponding to processing of chromatin from a biological particle (e.g., cell, cell bead, or cell nucleus), and panel 1450 shows a workflow corresponding to processing of an gRNA molecule or derivative thereof from the biological particle (e.g., cell, cell bead, or cell nucleus).

As shown in panel 1400, in bulk solution, chromatin included within a biological particle (e.g., cell, cell bead, or cell nucleus) is processed (e.g., as described herein) to provide a template nucleic acid fragment (e.g., tagmented fragment) 1404 comprising insert sequence 1408 (e.g., region of open chromatin) and a complement thereof, transposon end sequences 1406 and complements thereof, sequencing primer or portion thereof 1402 (e.g., an R1 sequence), sequencing primer or portion thereof 1410 (e.g., an R2 sequence), and gaps 1407. Template nucleic acid fragment 1404 may then be partitioned within a partition (e.g., a droplet or well, as described herein). Within the partition, the biological particle (e.g., cell, cell bead, or cell nucleus) comprising template nucleic acid fragment 1404 may be lysed, permeabilized, or otherwise processed to provide access to template nucleic acid fragment 1404 (and one or more RNA molecules) therein. The partition may include a bead (e.g., gel bead) 1416 coupled to nucleic acid barcode molecules 1418a and 1418b. Nucleic acid barcode molecule 1418a may comprise a flow cell adapter sequence 1420a (e.g., a P5 sequence), a barcode sequence 1422a, and a sequencing primer or portion thereof or complement thereof 1402′. Sequences 1420a and 1422a may be hybridized to complementary sequences 1420′ and 1422′, respectively. Sequence 1402′ may hybridize to sequence 1402 of template nucleic acid fragment 1404, or its complement, and sequence 1422′ may be ligated 1412 to sequence 1402 of template nucleic acid fragment 1404. In some instances, template nucleic acid fragment 1404 may be phosphorylated using a suitable kinase enzyme (e.g., polynucleotide kinase (PNK), such as T4 PNK). In some instances, PNK and ATP may be added in bulk in the tagmentation (e.g., ATAC) reaction and/or prior to partitioning a biological particle (e.g., cell, cell bead, or cell nucleus), or a plurality thereof. 15U of PNK with 1 mM of ATP may be spiked into the tagmentation reaction. For example, less than 95U of PNK may be spiked into the tagmentation reaction. The contents of the partition may then be recovered in bulk solution (e.g., a droplet may be broken) to provide the partially double-stranded nucleic acid molecule comprising nucleic acid barcode molecule 1418a attached to template nucleic acid fragment 1404 in bulk solution. In bulk solution, gaps 1407 may be filled 1424 via a gap filling extension process (e.g., using a DNA polymerase) to provide a double-stranded nucleic acid molecule. This molecule may undergo amplification (e.g., PCR) 1426 to provide a double-stranded amplification product 1428 that includes sequences of the nucleic acid barcode molecule 1418a, the original chromatin molecule, and, optionally, an additional sequence 1430 that may be a flow cell adapter sequence (e.g., a P7 sequence). Gaps may be filled in the partition prior to bulk processing.

In parallel to the chromatin workflow of panel 1400, a gRNA molecule or derivative thereof of the same biological particle (e.g., cell, cell bead, or cell nucleus) may be processed. The process shown in panel 1450 is the same as that shown in panel 1250 of FIG. 12C, however, any other processing scheme described herein may be used. In the figure, bead 1416 and 1216b may be the same bead (e.g., a multifunctional bead).

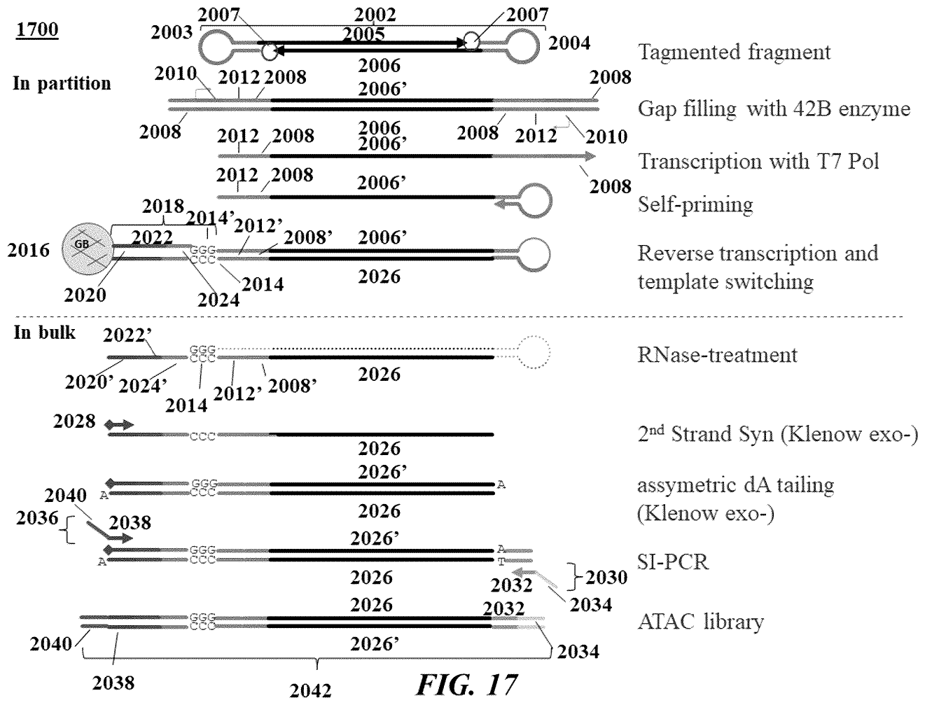

In another example, a biological particle (e.g., cell, cell bead, or cell nucleus) comprising chromatin and one or more gRNA molecules or derivatives thereof is provided. The chromatin in the biological particle (e.g., cell, cell bead, or cell nucleus) may be processed to provide a first template nucleic acid fragment derived from the chromatin (e.g., a tagmented fragment, as described herein). The chromatin may be processed in bulk solution. A gRNA molecule or derivative thereof may optionally be processed to provide a second template nucleic acid fragment derived from the gRNA molecule (e.g., an additional nucleic acid fragment, as described herein). The gRNA molecule may be processed within a partition. The second template nucleic acid fragment derived from the gRNA molecule may be processed according to the preceding examples. The configuration of the first template nucleic acid fragment may be at least partially dependent on the structure of the transposase-nucleic acid complex used to generate the first template nucleic acid fragment. For example, a transposase-nucleic acid complex such as that shown in FIG. 9 may be used to prepare the first template nucleic acid fragment. Relative to the preceding examples, the polarities of the transposase-nucleic acid may be reversed such that sequencing primers (e.g., R1 and R2 sequencing primers) are not directly linked to the chromatin (see, e.g., FIG. 17). The first template nucleic acid fragment may be at least partially double-stranded. The first template nucleic acid fragment may comprise a double-stranded region comprising sequences of chromatin of the biological particle (e.g., cell, cell bead, or cell nucleus). A first end of a first strand of the double-stranded region may be linked to a first transposon end sequence. A first end of the second strand of the double-stranded region, which end is opposite the first end of the first strand, may be linked to a second transposon end sequence. The second transposon end sequence may be the same as or different from the first transposon end sequence. The first transposon end sequence may be hybridized to a first complementary sequence, which first complementary sequence may not be linked to a second end of the second strand of the double-stranded region of the first template nucleic acid fragment. The first complementary sequence may be linked to a first sequencing primer or portion thereof. Similarly, the second transposon end sequence may be hybridized to a second complementary sequence, which second complementary sequence may not be linked to a second end of the first strand of the double-stranded region of the first template nucleic acid fragment. The second complementary sequence may be linked to a second sequencing primer or portion thereof. In other words, the first template nucleic acid fragment may comprise one or more gaps. In some cases, the one or more gaps may be approximately 9 bp in length each. For example, one or more gaps may be at least about 1, 2, 3, 4, 5, 6, 7, 8, 9, 10, or more bp in length. For example, one or more gaps may be at most about 10, 9, 8, 7, 6, 5, 4, 3, 2, or 1 bp in length. The first sequencing primer or portion thereof may be the same as or different from the second sequencing primer or portion thereof. In some cases, the first sequencing primer or portion thereof may be an R1 sequence or portion thereof, and the second sequencing primer or portion thereof may be an R2 sequence or portion thereof.

The biological particle (e.g., cell, cell bead, or cell nucleus) comprising the first template nucleic acid fragment (e.g., tagmented fragment) may be co-partitioned with one or more reagents into a partition of a plurality of partitions (e.g., as described herein). The partition may be, for example, a droplet or well. The partition may comprise one or more beads (e.g., as described herein). A bead (e.g., gel bead) of the one or more beads may comprise a first plurality of nucleic acid barcode molecules. A nucleic acid barcode molecule of the first plurality of nucleic acid barcode molecules may comprise a flow cell adapter sequence (e.g., P5 sequence), a barcode sequence, and a sequencing primer or portion thereof (e.g., R1 sequence or portion thereof, or a complement thereof). The sequencing primer or portion thereof may be complementary to a sequence of the first template nucleic acid fragment. The flow cell adapter sequence and/or barcode sequence may be hybridized to their complementary sequences. The same bead or another bead may comprise a second plurality of nucleic acid barcode molecules. A nucleic acid barcode molecule of the second plurality of nucleic acid barcode molecules may comprise a sequencing primer or portion thereof (e.g., an R1 sequence or portion thereof, or complement thereof), a barcode sequence, a unique molecular identifier sequence, and a capture sequence.

Within the partition, the gRNA molecule or derivative thereof may be processed to provide the second template nucleic acid fragment (e.g., as described herein).

Within the partition, the biological particle (e.g., cell, cell bead, or cell nucleus) may be lysed or permeabilized to provide access to the first and/or second template nucleic acid fragments therein (e.g., as described herein). The second template nucleic acid fragment may be generated after the biological particle (e.g., cell, cell bead, or cell nucleus) is lysed or permeabilized.

The first and second template nucleic acid fragments may undergo processing within the partition. Within the partition, a sequencing primer or portion thereof of the first template nucleic acid fragment corresponding to the chromatin of the biological particle (e.g., cell, cell bead, or cell nucleus) may hybridize to a sequencing primer or portion thereof of the nucleic acid barcode molecule of the first plurality of nucleic acid barcode molecules. The sequencing primer or portion thereof of the nucleic acid barcode molecule may then be ligated (e.g., using a ligase) to a transposon end sequence of the first template nucleic acid fragment, or a complement thereof to provide a partially double-stranded nucleic acid molecule corresponding to the chromatin of the biological particle (e.g., cell, cell bead, or cell nucleus).

Within the partition, the gRNA molecule or derivative thereof (e.g., second template nucleic acid fragment) may undergo processing to provide a barcoded nucleic acid molecule (e.g., as described herein). For example, a gRNA molecule or derivative thereof may undergo a ligation process to couple the gRNA molecule or derivative thereof to a nucleic acid barcode molecule of the second plurality of nucleic acid barcode molecules, thereby providing a barcoded nucleic acid molecule comprising sequences of the nucleic acid barcode molecule as well as sequences of the gRNA molecule or derivative thereof. In another example, a sequence of a gRNA molecule or derivative thereof may hybridize to a sequence of a primer molecule, after which a primer extension reaction may be performed to generate an extended primer product comprising sequences of the primer molecule and sequences complementary to the gRNA molecule or derivative thereof. The extended primer product may be ligated to a nucleic acid barcode molecule of the second plurality of nucleic acid barcode molecules to provide a barcoded nucleic acid molecule. Alternatively, a sequence of the extended primer product, such as an overhang sequence, may hybridize to a sequence of the nucleic acid barcode molecule to provide the barcoded nucleic acid molecule. In another example, two primer molecules may be used, one of which may comprise a sequence configured to hybridize to a first sequence of the gRNA molecule or derivative thereof and the other of which may comprise a sequence configured to hybridize to a second sequence of the gRNA molecule or derivative thereof. Following hybridization, primer extension reactions may be performed to provide a functionalized product comprising sequences of the gRNA molecule or derivative thereof, and complements thereof, as well as sequences of the primer molecules, and complements thereof. A sequence of the functionalized product may hybridize or ligate to a sequence of a nucleic acid barcode molecule of the second plurality of nucleic acid barcode molecules, thereby providing a barcoded nucleic acid molecule. Additional processing of a barcoded nucleic acid molecule comprising sequences of a gRNA molecule or derivative thereof, or complements thereof, may optionally be performed in the partition. For example, one or more functional sequences may be introduced. An additional amplification process may or may not be performed within a partition. For example, exponential amplification may or may not be performed within a partition.

The partially double-stranded molecule corresponding to the chromatin of the biological particle (e.g., cell, cell bead, or cell nucleus) and the barcoded molecule corresponding to the gRNA molecule or derivative thereof (e.g., prepared as described above) of the biological particle (e.g., cell, cell bead, or cell nucleus) included within the partition of the plurality of partitions may be recovered from the partition. For example, the contents of the plurality of partitions may be pooled to provide the linear amplification products in a bulk solution.

Outside of the partition, the gaps in the partially double-stranded nucleic acid molecule corresponding to the chromatin may be filled using via a gap filling extension process (e.g., using a DNA polymerase). Gaps may be filled in the partition prior to bulk processing. The resultant gap-filled double-stranded nucleic acid molecule may be denatured to provide a single strand, which single strand may be subjected to conditions sufficient to perform one or more nucleic acid amplification reactions (e.g., PCR) to generate amplification products corresponding to the chromatin of the cell, cell bead, or cell nucleus. A nucleic acid amplification process may incorporate one or more additional sequences, such as one or more additional flow cell adapter sequences. The barcoded molecule corresponding to the gRNA molecule or derivative thereof may also be processed and amplified according to the preceding examples.

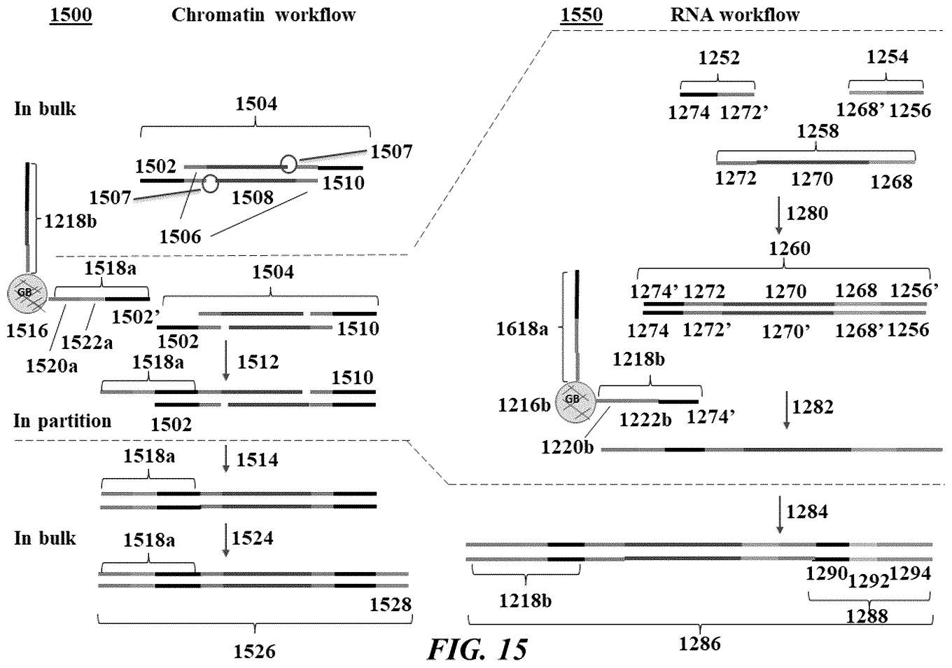

FIG. 15 shows an example schematic corresponding to the preceding example. Panel 1500 shows a workflow corresponding to processing of chromatin from a biological particle (e.g., cell, cell bead, or cell nucleus), and panel 1550 shows a workflow corresponding to processing of an gRNA molecule or derivative thereof from the biological particle (e.g., cell, cell bead, or cell nucleus).

As shown in panel 1500, in bulk solution, chromatin included within a cell, cell bead, or cell nucleus is processed (e.g., as described herein) to provide a template nucleic acid fragment (e.g., tagmented fragment) 1504 comprising insert sequence 1508 (e.g., region of open chromatin) and a complement thereof, transposon end sequences 1506 and complements thereof, sequencing primer or portion thereof 1502 (e.g., an R1 sequence), sequencing primer or portion thereof 1510 (e.g., an R2 sequence), and gaps 1507. Template nucleic acid fragment 1504 may then be partitioned within a partition (e.g., a droplet or well, as described herein). Within the partition, the cell, cell bead, or cell nucleus comprising template nucleic acid fragment 1504 may be lysed, permeabilized, or otherwise processed to provide access to template nucleic acid fragment 1504 (and one or more RNA molecules) therein. The partition may include a bead (e.g., gel bead) 1516 coupled to nucleic acid barcode molecules 1518a and 1518b. Nucleic acid barcode molecule 1518a may comprise a flow cell adapter sequence 1520a (e.g., a P5 sequence), a barcode sequence 1522a, and a sequencing primer or portion thereof or complement thereof 1502′. Sequence 1502′ may hybridize to sequence 1502 of template nucleic acid fragment 1504, or its complement. Sequence 1502′ may then be ligated 1512 to a transposon end sequence 1506 of template nucleic acid fragment 1504. In some instances, 1504 may be phosphorylated using a suitable kinase enzyme (e.g., polynucleotide kinase (PNK), such as T4 PNK). In some instances, PNK and ATP may be added in bulk in the tagmentation (e.g., ATAC) reaction and/or prior to partitioning a cell, cell bead, or cell nucleus, or plurality thereof. 15U of PNK with 1 mM of ATP may be spiked into the tagmentation reaction. For example, less than 95U of PNK may be spiked into the tagmentation reaction. The contents of the partition may then be recovered in bulk solution (e.g., a droplet may be broken) to provide the partially double-stranded nucleic acid molecule comprising nucleic acid barcode molecule 1518a attached to template nucleic acid fragment 1504 in bulk solution. In bulk solution, gaps 1507 may be filled 1514 via a gap filling extension process (e.g., using a DNA polymerase) and the molecule extended from sequence 1502 to provide a double-stranded nucleic acid molecule. This molecule may undergo amplification (e.g., PCR) 1524 to provide a double-stranded amplification product 1526 that includes sequences of the nucleic acid barcode molecule 1518a, the original chromatin molecule, and, optionally, an additional sequence 1528 that may be a flow cell adapter sequence (e.g., a P7 sequence). Gaps may be filled in the partition prior to bulk processing.

In parallel to the chromatin workflow of panel 1500, a gRNA molecule or derivative thereof of the same biological particle (e.g., cell, cell bead, or cell nucleus) may be processed. The process shown in panel 1550 is the same as that shown in panel 1250 of FIG. 12C, however, any other processing scheme described herein may be used. In the figure, bead 1516 and 1216b may be the same bead (e.g., a multifunctional bead).

In another example, a biological particle (e.g., cell, cell bead, or cell nucleus) comprising chromatin and one or more gRNA molecules or derivatives thereof is provided. The chromatin in the biological particle (e.g., cell, cell bead, or cell nucleus) may be processed to provide a first template nucleic acid fragment derived from the chromatin (e.g., a tagmented fragment, as described herein). The chromatin may be processed in bulk solution. A gRNA molecule or derivative thereof may be processed to provide a second template nucleic acid fragment derived from a gRNA molecule (e.g., as described herein). The gRNA molecule may be processed within a partition. The configuration of the first template nucleic acid fragment may be at least partially dependent on the structure of the transposase-nucleic acid complex used to generate the first template nucleic acid fragment. For example, a transposase-nucleic acid complex such as that shown in FIG. 9 may be used to prepare the first template nucleic acid fragment. The first template nucleic acid fragment may be at least partially double-stranded. The first template nucleic acid fragment may comprise a double-stranded region comprising sequences of chromatin of the biological particle (e.g., cell, cell bead, or cell nucleus). A first end of a first strand of the double-stranded region may be linked to a first transposon end sequence, which first transposon end sequence may be linked to a first sequencing primer or portion thereof. A first end of the second strand of the double-stranded region, which end is opposite the first end of the first strand, may be linked to a second transposon end sequence, which second transposon end sequence may be linked to a second sequencing primer or portion thereof. The second transposon end sequence may be the same as or different from the first transposon end sequence. The first sequencing primer or portion thereof may be the same as or different from the second sequencing primer or portion thereof. In some cases, the first sequencing primer or portion thereof may be an R1 sequence or portion thereof, and the second sequencing primer or portion thereof may be an R2 sequence or portion thereof. The first transposon end sequence may be hybridized to a first complementary sequence, which first complementary sequence may not be linked to a second end of the second strand of the double-stranded region of the first template nucleic acid fragment. Similarly, the second transposon end sequence may be hybridized to a second complementary sequence, which second complementary sequence may not be linked to a second end of the first strand of the double-stranded region of the first template nucleic acid fragment. In other words, the first template nucleic acid fragment may comprise one or more gaps. In some cases, the one or more gaps may be approximately 9 bp in length each. For example, one or more gaps may be at least about 1, 2, 3, 4, 5, 6, 7, 8, 9, 10, or more bp in length. For example, one or more gaps may be at most about 10, 9, 8, 7, 6, 5, 4, 3, 2, or 1 bp in length. The second template nucleic acid fragment (e.g., an additional template nucleic acid fragment) may comprise a sequence of an RNA molecule of the biological particle (e.g., cell, cell bead, or cell nucleus) and a sequence hybridized to a primer molecule (e.g., a capture nucleic acid molecule). For example, the second template nucleic acid fragment may comprise a sequence of an RNA molecule of the biological particle (e.g., cell, cell bead, or cell nucleus) and a polyA sequence hybridized to a polyT sequence of a primer molecule. The primer molecule may also comprise an additional primer sequence.

The biological particle (e.g., cell, cell bead, or cell nucleus) comprising the first template nucleic acid fragment (e.g., tagmented fragment) may be co-partitioned with one or more reagents into a partition of a plurality of partitions (e.g., as described herein). The partition may be, for example, a droplet or well. The partition may comprise one or more beads (e.g., as described herein). A bead (e.g., gel bead) of the one or more beads may comprise a first plurality of nucleic acid barcode molecules. A nucleic acid barcode molecule of the first plurality of nucleic acid barcode molecules may comprise a flow cell adapter sequence (e.g., P5 sequence), a barcode sequence, and an overhang sequence. The partition may also comprise a splint sequence comprising a sequence complementary to the overhang sequence and a sequencing primer or portion thereof that may be complementary to a sequence of the first template nucleic acid fragment. A bead of the one or more beads may also comprise a second plurality of nucleic acid barcode molecules. A nucleic acid barcode molecule of the second plurality of nucleic acid barcode molecules may comprise a flow cell adapter sequence (e.g., P5 sequence), a barcode sequence, a sequencing primer or portion thereof (e.g., R1 sequence or portion thereof, or a complement thereof), a UMI sequence, and a capture sequence (e.g., a polyG sequence or a polydT sequence). In some cases, the first plurality of nucleic acid barcode molecules and the second plurality of nucleic acid barcode molecules may be coupled to the same bead, and the partition may comprise a single bead.

Within the partition, the gRNA molecule may be processed to provide the second template nucleic acid fragment (e.g., as described herein).

Within the partition, the biological particle (e.g., cell, cell bead, or cell nucleus) may be lysed or permeabilized to provide access to the first and/or second template nucleic acid fragments therein (e.g., as described herein). The second template nucleic acid fragment may be generated after the biological particle (e.g., cell, cell bead, or cell nucleus) is lysed or permeabilized.

The first and second template nucleic acid fragments may undergo processing within the partition. Within the partition, a sequencing primer or portion thereof of the first template nucleic acid fragment corresponding to the chromatin of the biological particle (e.g., cell, cell bead, or cell nucleus) may hybridize to a complementary sequence of the sequencing primer or portion thereof in the splint sequence. The splint sequence may also hybridize to the overhang sequence of the nucleic acid barcode molecule of the first plurality of nucleic acid barcode molecules. The overhang sequence of the nucleic acid barcode molecule may then be ligated (e.g., using a ligase) to a sequencing primer or portion thereof of the first template nucleic acid fragment. The resultant partially double-stranded nucleic acid molecule may comprise the barcode sequence as well as one or more gaps.

Within the partition, the gRNA molecule or derivative thereof (e.g., second template nucleic acid fragment) may undergo processing to provide a barcoded nucleic acid molecule (e.g., as described herein). For example, a gRNA molecule or derivative thereof may undergo a ligation process to couple the gRNA molecule or derivative thereof to a nucleic acid barcode molecule of the second plurality of nucleic acid barcode molecules, thereby providing a barcoded nucleic acid molecule comprising sequences of the nucleic acid barcode molecule as well as sequences of the gRNA molecule or derivative thereof. In another example, a sequence of a gRNA molecule or derivative thereof may hybridize to a sequence of a primer molecule, after which a primer extension reaction may be performed to generate an extended primer product comprising sequences of the primer molecule and sequences complementary to the gRNA molecule or derivative thereof. The extended primer product may be ligated to a nucleic acid barcode molecule of the second plurality of nucleic acid barcode molecules to provide a barcoded nucleic acid molecule. Alternatively, a sequence of the extended primer product, such as an overhang sequence, may hybridize to a sequence of the nucleic acid barcode molecule to provide the barcoded nucleic acid molecule. In another example, two primer molecules may be used, one of which may comprise a sequence configured to hybridize to a first sequence of the gRNA molecule or derivative thereof and the other of which may comprise a sequence configured to hybridize to a second sequence of the gRNA molecule or derivative thereof. Following hybridization, primer extension reactions may be performed to provide a functionalized product comprising sequences of the gRNA molecule or derivative thereof, and complements thereof, as well as sequences of the primer molecules, and complements thereof. A sequence of the functionalized product may hybridize or ligate to a sequence of a nucleic acid barcode molecule of the second plurality of nucleic acid barcode molecules, thereby providing a barcoded nucleic acid molecule. Additional processing of a barcoded nucleic acid molecule comprising sequences of a gRNA molecule or derivative thereof, or complements thereof, may optionally be performed in the partition. For example, one or more functional sequences may be introduced. An additional amplification process may or may not be performed within a partition. For example, exponential amplification may or may not be performed within a partition.

The partially double-stranded nucleic acid molecule corresponding to the chromatin of the biological particle (e.g., cell, cell bead, or cell nucleus) and the barcoded molecule corresponding to the gRNA molecule or derivative thereof of the biological particle (e.g., cell, cell bead, or cell nucleus) included within the partition of the plurality of partitions may be recovered from the partition. For example, the contents of the plurality of partitions may be pooled to provide the partially double-stranded nucleic acid molecule and the barcoded molecule corresponding to the gRNA molecule or derivative thereof in a bulk solution.

Outside of the partition, the gaps in the partially double-stranded nucleic acid molecule corresponding to the chromatin may be filled using via a gap filling extension process (e.g., using a DNA polymerase or reverse transcriptase). The DNA polymerase may lack strand displacement activity. The resultant gap-filled double-stranded nucleic acid molecule may be denatured to provide a single strand, which single strand may be subjected to conditions sufficient to perform one or more nucleic acid amplification reactions (e.g., PCR) to generate amplification products corresponding to the chromatin of the biological particle (e.g., cell, cell bead, or cell nucleus). A nucleic acid amplification process may incorporate one or more additional sequences, such as one or more additional flow cell adapter sequences.

Outside of the partition, the barcoded molecule corresponding to the gRNA molecule or derivative thereof may be subjected to fragmentation, end repair, a dA tailing process, tagmentation, or a combination thereof. An additional primer sequence (e.g., a sequencing primer or portion thereof, such as an R2 sequence) may then be ligated to the resultant molecule. A nucleic acid amplification reaction (e.g., PCR) may then be performed to generate one or more amplification products corresponding to the gRNA molecule or derivative thereof. A nucleic acid amplification process may incorporate one or more additional sequences, such as one or more additional flow cell adapter sequences.

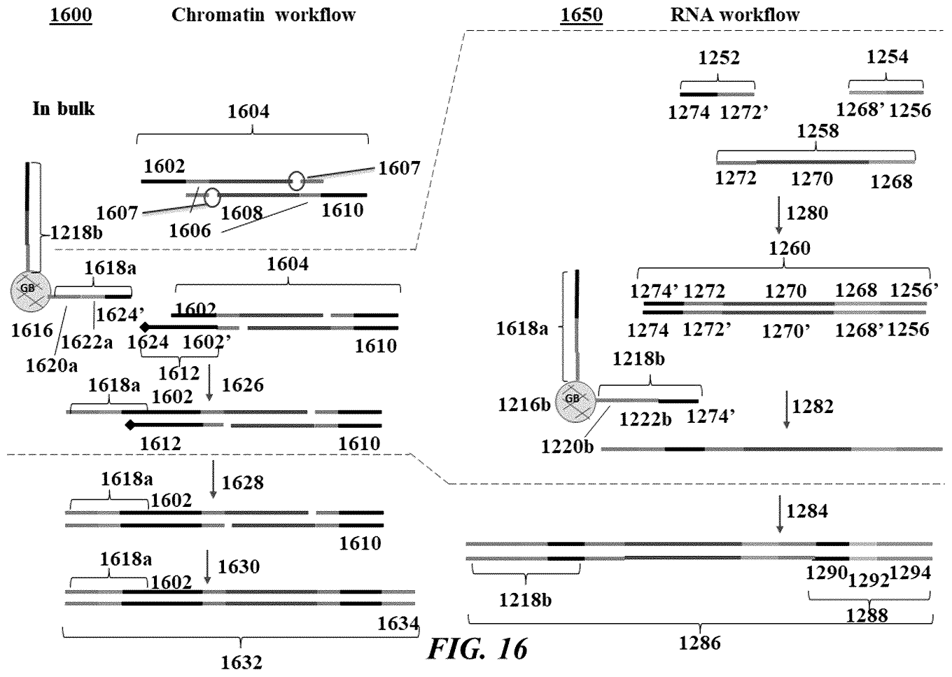

FIG. 16 shows an example schematic corresponding to the preceding example. Panel 1600 shows a workflow corresponding to processing of chromatin from a biological particle (e.g., cell, cell bead, or cell nucleus), and panel 1650 shows a workflow corresponding to processing of an gRNA molecule or derivative thereof from the biological particle (e.g., cell, cell bead, or cell nucleus).

As shown in panel 1600, in bulk solution, chromatin included within a cell, cell bead, or cell nucleus is processed (e.g., as described herein) to provide a template nucleic acid fragment (e.g., tagmented fragment) 1604 comprising insert sequence 1608 (e.g., region of open chromatin) and a complement thereof, transposon end sequences 1606 and complements thereof, sequencing primer or portion thereof 1602 (e.g., an R1 sequence), sequencing primer or portion thereof 1610 (e.g., an R2 sequence), and gaps 1607. Template nucleic acid fragment 1604 may then be partitioned within a partition (e.g., a droplet or well, as described herein). Within the partition, the cell, cell bead, or cell nucleus comprising template nucleic acid fragment 1604 may be lysed, permeabilized, or otherwise processed to provide access to template nucleic acid fragment 1604 (and one or more RNA molecules) therein. The partition may comprise splint sequence 1612, which splint sequence may comprise a first sequence 1602′ that is complementary to sequencing primer or portion thereof 1602 and a second sequence 1624. Sequence 1624 may comprise a blocking group (e.g., a 3′ blocking group), which blocking group may prevent extension by reverse transcription. The partition may also include a bead (e.g., gel bead) 1616 coupled to nucleic acid barcode molecules 1618a and 1612b. Nucleic acid barcode molecule 1618a may comprise a flow cell adapter sequence 1620a (e.g., a P5 sequence), a barcode sequence 1622a, and an overhang sequence 1624′ that is complementary to sequence 1624 of the splint sequence. Sequence 1624 may hybridize to sequence 1624′ to provide a partially double-stranded nucleic acid molecule comprising the sequences of nucleic acid barcode molecule 1618a and the template nucleic acid fragment 1604. Sequence 1624′ of nucleic acid barcode molecule 1618a may be ligated (e.g., using a ligase) 1626 to sequence 1602 of template nucleic acid fragment 1604. In some instances, 1604 may be phosphorylated using a suitable kinase enzyme (e.g., polynucleotide kinase (PNK), such as T4 PNK). In some instances, PNK and ATP may be added in bulk in the tagmentation reaction (e.g., ATAC) and/or prior to partitioning a cell, cell bead, or cell nucleus, or plurality thereof. 15U of PNK with 1 mM of ATP may be spiked into the tagmentation reaction. For example, less than 95U of PNK may be spiked into the tagmentation reaction. The contents of the partition may then be recovered in bulk solution (e.g., a droplet may be broken) to provide the partially double-stranded nucleic acid molecule comprising nucleic acid barcode molecule 1618a attached to template nucleic acid fragment 1604 in bulk solution. In bulk solution, gaps 1607 may be filled 1628 via a gap filling extension process (e.g., using a DNA polymerase) to provide a double-stranded nucleic acid molecule. This molecule may undergo amplification (e.g., PCR) 1630 to provide a double-stranded amplification product 1632 that includes sequences of the nucleic acid barcode molecule 1618a, the original chromatin molecule, and, optionally, an additional sequence 1634 that may be a flow cell adapter sequence (e.g., a P7 sequence). Gaps may be filled in the partition prior to bulk processing.

In parallel to the chromatin workflow of panel 1600, a gRNA molecule or derivative thereof of the same biological particle (e.g., cell, cell bead, or cell nucleus) may be processed. The process shown in panel 1650 is the same as that shown in panel 1250 of FIG. 12C, however, any other processing scheme described herein may be used. In the figure, bead 1616 and 1216b may be the same bead (e.g., a multifunctional bead).

In some cases, chromatin processing may comprise reverse transcription of a DNA molecule to provide an RNA molecule (e.g., using a reverse transcriptase). This process may be performed within a partition (e.g., as described herein). The RNA molecule may then be reverse transcribed (e.g., using a reverse transcriptase) to generate a cDNA molecule (e.g., within a partition). The cDNA molecule may undergo further processing (e.g., within a partition) to provide a derivative of the cDNA molecule. The cDNA molecule or derivative thereof may be recovered from a partition (e.g., by pooling the contents of a plurality of partitions including a partition in which chromatin processing is performed, such as a plurality of wells or droplets). Processing of a gRNA molecule or derivative thereof may be performed as described herein in parallel.

Processing chromatin according to these scheme may comprise processing an open chromatin structure of a nucleic acid sample with a transposase (e.g., included within a transposase-nucleic acid complex) to provide the DNA molecule (e.g., as described herein). A transposase-nucleic acid complex may comprise one or more transposon end oligonucleotide molecules, which transposon end oligonucleotide molecules comprise hairpin molecules. An example of such a transposase-nucleic acid complex is shown in FIG. 11. A nucleic acid molecule processed using a transposase-nucleic acid complex comprising one or more hairpin molecules may be a tagmented fragment comprising a double-stranded region comprising sequences corresponding to the nucleic acid molecule (e.g., chromatin) of the biological particle (e.g., cell, cell bead, or cell nucleus) from which it originates or is derived, as well as one or more hairpin molecules appended to either end of the double-stranded region. For example, the double-stranded region may comprise a first hairpin molecule at one end and a second hairpin molecule at a second end. Generally, only one end of a hairpin molecule may be attached to the double-stranded region, such that the tagmented fragment comprises a gap at either end. For example, a hairpin molecule may be attached to a 3′ end of the double-stranded region. The hairpin molecule may comprise a promoter sequence, such as a T7 promoter sequence, and/or a UMI sequence.

Within the partition, the nucleic acid molecule (e.g., tagmented fragment) may undergo a gap filling process with a reverse transcriptase. The reverse transcriptase enzyme may be a mutant reverse transcriptase enzyme such as, but not limited to, Moloney Murine Leukemia Virus (MMLV) reverse transcriptase. In one aspect, the reverse transcriptase is a mutant MMLV reverse transcriptase such as, but not limited to, enzyme “42B” (see, US Patent Publication No. 20180312822). Enzyme 42B may reduce inhibition of reverse transcription of mRNAs from a single cell due to one or more unknown components present in cell lysate of the single cell when prepared in reaction volumes of, e.g., less than 1 nanoliter (nL). Enzyme 42B as compared to a commercially available mutant MMLV RT enzyme (CA-MMLV) may show improved reverse transcriptase activity. Such a process may generate a double-stranded nucleic acid molecule comprising the double-stranded region corresponding to the nucleic acid molecule (e.g., chromatin) of the biological particle (e.g., cell, cell bead, or cell nucleus) from which it is derived, the sequences of the hairpin molecules at either end of the double-stranded region, and sequences complementary to the sequences of the hairpin molecules. The double-stranded nucleic acid molecule may then undergo transcription with a T7 polymerase, which process begins at an end of a T7 promoter sequences of a hairpin molecules. Both strands may be transcribed in this manner to provide two nucleic acid strands each comprising the T7 promoter sequence, and a complement thereof; one or more transposon end sequences, and one or more complements thereof; and a sequence of the original nucleic acid molecule of the biological particle (e.g., cell, cell bead, or cell nucleus). The strands may also comprise one or more spacer, UMI, or other sequences (e.g., from the hairpin molecules). A strand may then undergo a self-priming process in which the transposon end sequence and complement thereof of a hairpin molecule hybridize to one another to regenerate a hairpin molecule at an end of the strand. The hairpin molecule may serve as the priming site for reverse transcription. A reverse transcriptase process may then be performed (e.g., using a reverse transcriptase). Before, during, or after this process, a sequence may be appended to the end of the molecule, which sequence may be a polyC sequence. A template switching oligonucleotide comprising a sequence complementary to the appended sequence (e.g., a polyG sequence) may hybridize to the appended sequence. The template switching oligonucleotide may comprise a UMI sequence (e.g., a second UMI sequence that may index transcripts that undergo template switching), a barcode sequence, and/or a priming sequence such as a sequencing primer sequence or portion thereof (e.g., an R1 or R2 sequence, or portion thereof). The template switching oligonucleotide may be attached to a bead (e.g., a gel bead) included within the partition. For example, the template switching oligonucleotide may be a nucleic acid barcode molecule of a plurality of nucleic acid barcode molecules attached to the bead (e.g., as described herein). The resultant partially double-stranded nucleic acid molecule may comprise a hairpin moiety; sequences corresponding to the original nucleic acid molecule of the cell, cell bead, or cell nucleus; and the sequences of the template switching oligonucleotide, including a barcode sequence (see, e.g., FIG. 17).

The partially double-stranded nucleic acid molecule may be released from the partition (e.g., droplet or well). Releasing materials from the partition may comprise breaking or disrupting a droplet. The contents of multiple partitions of the plurality of partitions may be pooled together to provide a bulk solution for further processing. Nucleic acid molecules (e.g., partially double-stranded nucleic acid molecule) of the partitions of the plurality of partitions may each be differentially barcoded such that the nucleic acid molecule of each such partition comprises a different barcode sequence.

Outside of the partition, the partially double-stranded nucleic acid molecule may be partially denatured to provide a single-stranded molecule (e.g., a single-stranded cDNA molecule). An RNase treatment may be used to remove the hairpin molecule as well as the shorter strand (e.g., the RNA sequence) of the partially double-stranded nucleic acid molecule. The single-stranded molecule remaining may include the template switching oligonucleotide comprising the barcode sequence and, optionally, UMI sequence. A primer molecule comprising a priming sequence complementary to the priming sequence of the template switching oligonucleotide may be provided and may hybridize to the priming sequence of the template switching oligonucleotide. The priming sequence of the primer molecule may be a 5′-blocked priming sequence. A polymerase with dA tailing activity (e.g., a Klenow fragment having 5′→3′ polymerase activity, such as an exo-Klenow fragment lacking exonuclease activity) may be used to generate a second nucleic acid strand. The resultant second strand may be dA tailed. The first strand may also be dA tailed. However, if a 5′-blocking priming sequence is used in the preceding processes, the dA tail appended to the first strand may not be available as a hybridization site for another moiety. Instead, a priming sequence comprising a sequencing primer (e.g., an R1 sequence or complement thereof) and a flow cell adapter sequence (e.g., a P5 sequence or complement thereof) may hybridize to a complementary sequence of the double-stranded nucleic acid molecule. At the opposite end of the double-stranded nucleic acid molecule, the dA moiety appended to the end of the second strand may serve as a site for hybridization of a priming sequence comprising a dT moiety at an end, a sequencing primer (e.g., an R2 sequence or complement thereof), and a flow cell adapter sequence (e.g., a P7 sequence or complement thereof). The double-stranded nucleic acid molecule may then be subjected to conditions sufficient to perform one or more nucleic acid amplification reactions (e.g., PCR) to provide amplification products corresponding to the original nucleic acid molecule of the cell, cell bead, or cell nucleus. The amplification products may comprise flow cell adapter sequences (e.g., P5 and P7 sequences) at either end to facilitate sequencing (e.g., as described herein).

The method provided herein may overcome certain challenges of performing reverse transcription within partitions. For example, reverse transcriptase may have a DNA-dependent DNA polymerase activity, and/or terminal transferase activities. The latter may result in generation of variable overhangs under certain reaction conditions. In the methods provided herein, every insertion site may be provided a T7 promoter, averting losses that may otherwise be encountered via R1-R1 and R2-R2 interactions. Moreover, both mRNA and chromatin-derived fragments may be barcoded using the same biochemistry (e.g., RT template switching). Performance of linear amplification of both such strands of a nucleic acid molecule may provide strand awareness and introduce a new dimension for, e.g., ATAC-seq processes. Further, this method may enable isothermal linear amplification of transposase derived nucleic acid fragments within partitions. Notably, this method may be combined with any of the RNA workflows described elsewhere herein.

FIG. 17 shows a workflow 1700 corresponding to the preceding example. Workflow 1700 may be performed in parallel with a gRNA workflow, such as a gRNA workflow described and/or shown anywhere herein. Multiple beads, each comprising nucleic acid barcode molecules configured for analysis of DNA or RNA molecules, may be included within a partition. Alternatively, a single bead (e.g., gel bead) comprising nucleic acid barcode molecules configured for analysis of both DNA and RNA molecules (e.g., as described herein) may be included within a partition. The single bead (e.g., in a single partition) may comprise a plurality of identical nucleic acid barcode molecules for both RNA and DNA analysis. In some cases, a single bead (e.g., within a single partition) comprises a first plurality of nucleic acid barcode molecules for DNA analysis and a second plurality of nucleic acid barcode molecules for RNA molecules, where the first and second plurality of nucleic acid barcode molecules comprise a common barcode sequence.

Template nucleic acid fragment (e.g., tagmented fragment) 2002 may be prepared (e.g., using a transposase-nucleic acid complex such as that shown in FIG. 11) and provided in a partition (as described herein). Template nucleic acid fragment 2002 may comprise hairpin moieties 2003 and 2004 and target sequences 2005 and 2006. Template nucleic acid fragment 2002 also comprises gaps 2007. Gaps 2007 may be filled using a reverse transcriptase (e.g., a 42B enzyme), which process may result in the generation of a double-stranded nucleic acid molecule comprising the double-stranded region corresponding to the original nucleic acid molecule (e.g., chromatin) of the cell, cell bead, or cell nucleus comprising sequences 2005 and 2006 and sequences of the hairpin molecules 2003 and 2004. The double-stranded nucleic acid molecule may comprise transposon end sequences 2008, promoter (e.g., T7 promoter) sequences 2010, and UMI sequences 2012. The double-stranded nucleic acid molecule may then undergo transcription with a T7 polymerase, which process begins at an end of a T7 promoter sequences of a hairpin molecule. Both strands may be transcribed in this manner to provide two nucleic acid strands. FIG. 17 shows one such strand comprising T7 promoter sequence 2010, and a complement thereof; one or more transposon end sequences 2008, and one or more complements thereof; UMI sequence 2012, and a complement of a UMI sequence; and an RNA sequence 2006′ corresponding to sequence 2006 of the original nucleic acid molecule of the cell, cell bead, or cell nucleus. The strand may then undergo a self-priming process in which the transposon end sequence and complement thereof of hairpin molecule 2004 hybridize to one another to regenerate a hairpin molecule at an end of the strand. Regenerated hairpin molecule 2004 may serve as the priming site for reverse transcription. Reverse transcription and template switching may then be performed (e.g., using a reverse transcriptase). The reverse transcription process may append sequence 2014 (e.g., a polyC sequence) to the resultant cDNA molecule comprising cDNA sequence 2026 and sequences 2012′ and 2008′ that are complementary to sequences 2012 and 2008, respectively. The template switching process may comprise the use of a template switch oligonucleotide coupled to bead (e.g., gel bead) 2016 included within the partition. Bead (e.g., gel bead) 2016 may be coupled to nucleic acid barcode molecule 2018 that is the template switch oligonucleotide that comprises sequencing primer or portion thereof 2020, barcode sequence 2022, UMI sequence 2024, and a sequence 2014′ that is complementary to sequence 2014 (e.g., a polyG sequence). The resultant cDNA molecule may comprise a first strand comprising nucleic acid barcode molecule 2018 and RNA sequence 2006′ and a second strand comprising cDNA sequence 2026, appended sequence 2014, and sequences 2020′, 2022′, and 2024′ that are complementary to sequences 2020, 2022, and 2024, respectively.

The cDNA molecule may be released from the partition (e.g., droplet or well). Releasing materials from the partition may comprise breaking or disrupting a droplet. The contents of multiple partitions of the plurality of partitions may be pooled together to provide a bulk solution for further processing. Outside of the partition, the cDNA molecule may be treated with RNase to remove the hairpin molecule as well as the shorter strand (e.g., the RNA sequence) of the partially double-stranded nucleic acid molecule. The single-stranded molecule remaining may include sequences 2020′, 2022′, 2024′, 2014, 2012′, 2008′, and 2026. Primer molecule 2028 may then hybridize to sequence 2020′. Primer molecule 2028 may be a 5′-blocked priming sequence. A polymerase with dA tailing activity (e.g., a Klenow fragment having 5′→3′ polymerase activity, such as an exo-Klenow fragment lacking exonuclease activity) may be used to generate a second nucleic acid strand comprising sequence 2026′ that is complementary to cDNA sequence 2026. The resultant second strand may be dA tailed. The first strand may also be dA tailed at an end of sequence 2020′. However, if a 5′-blocking priming sequence is used in the preceding processes, the dA tail appended to the first strand may not be available as a hybridization site for another moiety. A priming sequence 2030 comprising a dT moiety, a sequencing primer (e.g., an R2 sequence or complement thereof) 2032 and a flow cell adapter sequence (e.g., a P7 sequence or complement thereof) 2034 may hybridize to the dA moiety of the double-stranded nucleic acid molecule. A priming sequence 2036 comprising a sequencing primer (e.g., an R1 sequence or complement thereof) 2038 and a flow cell adapter sequence (e.g., a P5 sequence or complement thereof) 2040 may hybridize to sequence 2028 of the double-stranded nucleic acid molecule. The double-stranded nucleic acid molecule may then be amplified to provide amplified product 2042, which amplification product may be subjected to further processing such as nucleic acid sequencing.

In an example, a nucleic acid molecule may be processed using a reverse transcriptase fill-in process coupled with a barcoding process. The nucleic acid molecule (e.g., DNA molecule) may derive from a biological particle (e.g., cell, cell bead, or cell nucleus). In some cases, the nucleic acid molecule may be included within the biological particle (e.g., cell, cell bead, or cell nucleus). The nucleic acid molecule may be chromatin. The biological particle (e.g., cell, cell bead, or cell nucleus) comprising the nucleic acid molecule may be included within the partition. For example, the biological particle (e.g., cell, cell bead, or cell nucleus) may be co-partitioned with one or more reagents (e.g., as described herein) into a partition (e.g., droplet or well). The biological particle (e.g., cell, cell bead, or cell nucleus) may be lysed or permeabilized (e.g., within a partition) to provide access to the nucleic acid molecule therein (e.g., as described herein).

A nucleic acid molecule processed according to the method provided herein may be a DNA molecule, such as chromatin. In some cases, the method may further comprise processing an open chromatin structure of the nucleic acid sample with a transposase (e.g., included within a transposase-nucleic acid complex) to provide the nucleic acid molecule. For example, a nucleic acid molecule (e.g., within a biological particle, e.g., cell, cell bead, or cell nucleus) may be contacted with a transposase-nucleic acid complex (e.g., as described herein). A transposase used in such a process may be, for example, a Tn5 transposase. A transposase-nucleic acid complex may have a structure such as that of any one of FIG. 9, 10, or 11. Subsequent to generation of a tagmented fragment (e.g., as described herein), the transposase of the transposase-nucleic acid complex may leave or be removed (e.g., displaced, for example, by an enzyme). Alternatively, the transposase may remain in place. The tagmented fragment may comprise sequences corresponding to the original nucleic acid molecule of the biological particle (e.g., cell, cell bead, or cell nucleus); transposon end sequences and sequences complementary thereto; and one or more sequencing primers or portions thereof. A splint sequence comprising a sequence complementary to a sequencing primer or portion thereof the tagmented fragment may hybridize to the sequencing primer or portion thereof. The splint sequence may be ligated to a transposon end sequence or complement thereof of the tagmented fragment (e.g., using a ligase). Prior to or after hybridization and/or ligation of the splint sequence, the tagmented fragment may be partitioned into a partition of a plurality of partitions (e.g., droplets of wells). The tagmented fragment may be co-partitioned with one or more reagents. The tagmented fragment may be included within a biological particle (e.g., cell, cell bead, or cell nucleus), which biological particle (e.g., cell, cell bead, or cell nucleus) may be lysed or permeabilized to provide access to the tagmented fragment therein (e.g., as described herein). A sequence of the splint sequence may then hybridize to a nucleic acid barcode molecule (e.g., a nucleic acid barcode molecule coupled to a bead, as described herein). The bead (e.g., gel bead) may comprise a plurality of nucleic acid barcode molecules, where a nucleic acid barcode molecule of the plurality of nucleic acid barcode molecules may comprise, for example, a flow cell adapter sequence, a barcode sequence, and a UMI sequence. The nucleic acid barcode molecule may also comprise an overhang sequence having sequence complementarity to a sequence of the splint sequence. The overhang sequence may hybridize to the sequence of the splint sequence. A transposase reserved in the tagmented fragment may block gap filling during these processes. The splint sequence may then be extended within the partition (e.g., using a reverse transcriptase).

Subsequent to the barcoding/template switching and extension (e.g., reverse transcription) processes, the contents of the partition of the plurality of partitions may be released from the partition (e.g., as described herein). Prior or subsequent to release of the contents of the partition, the nucleic acid barcode molecule may be ligated to the sequencing primer of the processed tagmented fragment. Outside of the partition, the nucleic acid barcode molecule may hybridize to the sequencing primer or portion thereof of the template nucleic acid fragment. If a transposase is reserved in the tagmented fragment, the transposase may leave the processed tagmented fragment (e.g., via a strand displacing polymerase) and the remaining gaps may be filled to provide a double-stranded nucleic acid molecule. Alternatively, gaps may be filled as described elsewhere herein. The double-stranded nucleic acid molecule may then be subjected to a nucleic acid amplification process (e.g., PCR, as described herein). Amplification may comprise incorporation of one or more additional sequences, such as one or more flow cell adapter sequences (e.g., P7 sequences).

In parallel, any useful process may be used to analyze a gRNA molecule or derivative thereof.

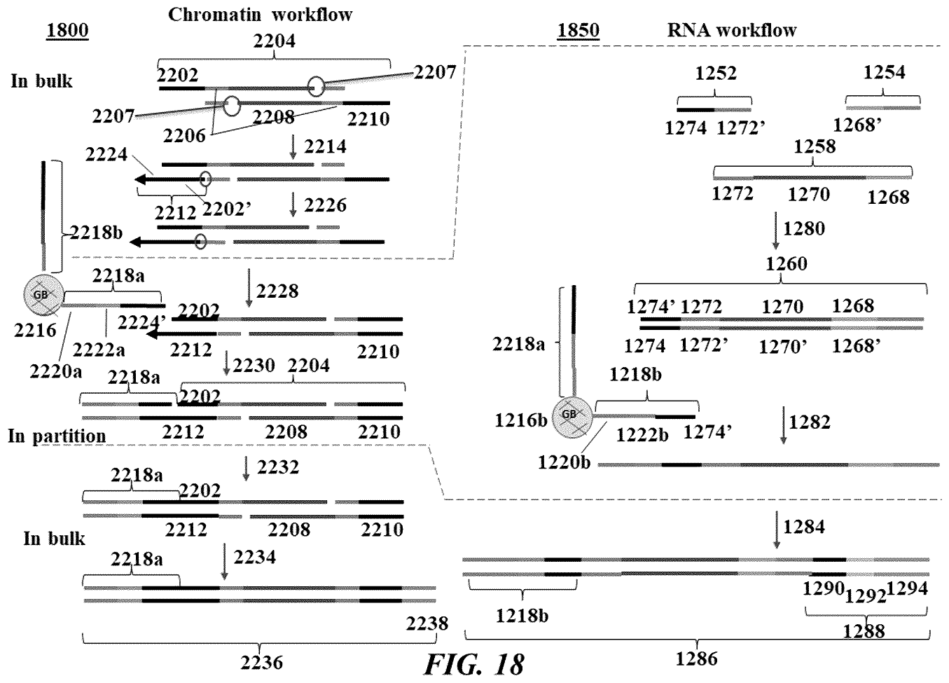

FIG. 18 shows an example schematic corresponding to the preceding example. Panel 1800 shows a workflow corresponding to processing of chromatin from a biological particle (e.g., cell, cell bead, or cell nucleus), and panel 2250 shows a workflow corresponding to processing of a gRNA molecule or derivative thereof from the biological particle (e.g., cell, cell bead, or cell nucleus). Multiple beads (e.g., gel beads), each comprising nucleic acid barcode molecules configured for analysis of DNA and/or RNA molecules, may be included within a partition. Alternatively, a single bead (e.g., gel bead) comprising nucleic acid barcode molecules configured for analysis of both DNA and RNA molecules (e.g., as described herein) may be included within a given partition. In some embodiments, a single bead (e.g., gel bead) comprising nucleic acid barcode molecules configured for analysis of both tagmented DNA fragments and nucleic acid molecules associated with perturbation agents (e.g., guide RNA molecules or a precursor, portion, or a derivative thereof, any of which may be associated with the gRNA or a gRNA-identifying barcode, or optionally, other components of a CRISPR-Cas system) (e.g., as described herein) may be included within a given partition.

As shown in panel 1800, in bulk solution, chromatin included within a biological particle (e.g., biological particle (e.g., cell, cell bead, or cell nucleus)) is processed (e.g., as described herein) to provide a template nucleic acid fragment (e.g., tagmented fragment) 2204 comprising insert sequence 2208 (e.g., region of open chromatin) and a complement thereof, transposon end sequences 2206 and complements thereof, sequencing primer or portion thereof 2202 (e.g., an R1 sequence), sequencing primer or portion thereof 2210 (e.g., an R2 sequence), and gaps 2207. The biological particle (e.g., cell, cell bead, or cell nucleus) comprising template nucleic acid fragment 2204 may be lysed, permeabilized, or otherwise processed to provide access to template nucleic acid fragment 2204 (and one or more RNA molecules) therein. Template nucleic acid fragment 2204 may be contacted with splint sequence 2212, which splint sequence may comprise a first sequence 2202′ that is complementary to sequencing primer or portion thereof 2202 and a second sequence 2224. Sequence 2224 may comprise a blocking group (e.g., a 3′ blocking group), which blocking group may prevent extension by reverse transcription. Sequence 2202′ may hybridize 2214 to sequence 2202 of template nucleic acid fragment 2204 to provide a partially double-stranded nucleic acid molecule comprising splint sequence 2212 and template nucleic acid fragment 2204. Sequence 2202′ may be ligated 2226 to the complement of transposon end sequence 2206 of template nucleic acid fragment 2204 (e.g., using a ligase). Template nucleic acid fragment 2204 attached to splint sequence 2212 may then be partitioned within a partition (e.g., droplet or well) within a plurality of partitions (e.g., as described herein). The partition may also include a bead (e.g., gel bead) 2216 coupled to nucleic acid barcode molecules 2218a and 2218b. Nucleic acid barcode molecule 2218a may comprise a flow cell adapter sequence 2220a (e.g., a P5 sequence), a barcode sequence 2222a, and an overhang sequence 2224′ that is complementary to sequence 2224 of the splint sequence 2212. Sequence 2224 may hybridize 2228 to sequence 2224′. Splint sequence 2212 may then be extended 2230 (e.g., using a reverse transcriptase or DNA polymerase) to provide sequences 2220a′ and 2222a′ that are complementary to sequences 2220a and 2222a of nucleic acid barcode molecule 2218a. Alternatively, sequence 2224 may hybridize to sequence 2224′ to provide a partially double-stranded nucleic acid molecule and nucleic acid barcode molecule 2218a may be ligated (e.g., using a ligase) to sequence 2202 of template nucleic acid fragment 2204. The contents of the partition may then be recovered in bulk solution (e.g., a droplet may be broken) to provide the partially double-stranded nucleic acid molecule comprising nucleic acid barcode molecule 2218a attached to splint sequence 2212 and template nucleic acid fragment 2204 in bulk solution. Sequence 2224′ of nucleic acid barcode molecule 2218a may be ligated (e.g., using a ligase) 2232 to sequence 2202 of template nucleic acid fragment 2204. In bulk solution, gaps 2207 may be filled 2234 via a gap filling extension process (e.g., using a DNA polymerase) to provide a double-stranded nucleic acid molecule. This molecule may also undergo amplification (e.g., PCR) to provide a double-stranded amplification product 2236 that includes sequences of the nucleic acid barcode molecule 2218a, the original chromatin molecule, and, optionally, an additional sequence 2238 that may be a flow cell adapter sequence (e.g., a P7 sequence). Gaps may be filled in the partition prior to bulk processing.

In parallel to the chromatin workflow of panel 1800, a gRNA molecule or derivative thereof of the same biological particle (e.g., cell, cell bead, or cell nucleus) may be processed. The process shown in panel 1850 is the same as that shown in panel 1250 of FIG. 12C, however, any other processing scheme described herein may be used. In the figure, bead 1816 and 1216b may be the same bead (e.g., a multifunctional bead).

The methods described herein may be applied to a single biological particle (e.g., cell, cell bead, or cell nucleus) or a collection of biological particles (e.g., cells, cell beads, or cell nuclei). For example, perturbations (e.g., perturbations corresponding to introduction of gRNA molecules or precursors thereof) may be applied to a single biological particle (e.g., cell, cell bead, or nucleus) or a population of biological particles (e.g., cells, cell beads, or cell nuclei). In an example, a plurality of gRNA molecules or precursors thereof encoding a plurality of different genomic edits (e.g., gRNA molecules or precursors thereof configured to target a plurality of different genes, a plurality of different regions of a same gene, or a combination thereof, and/or a randomized collection of gRNA molecules or precursors thereof) may be introduced to a plurality of biological particles (e.g., cells, cell beads, or cell nuclei). The plurality of biological particles may derive from the same source (e.g., a same subject or patient, such as from one or more nucleic acid samples collected from a same subject or patient) or from different sources (e.g., from different subjects or patients, such as from nucleic acid samples collected from different subjects, or from different samples collected from a same subject or patient). The plurality of biological particles may derive from a same cell line. The plurality of biological particles may comprise a plurality of different types of cells, cell beads, or cell nuclei and/or cells, cell beads, or cell nuclei that have undergone one or more different processing or sample preparation protocols. The plurality of gRNA molecules or precursors thereof may be introduced to the plurality of biological particles in a spatially resolved manner. For example, the plurality of gRNA molecules or precursors thereof may be introduced to the plurality of biological particles according to a pre-defined 2-dimensional or 3-dimensional pattern (e.g., as described herein). gRNA molecules or precursors thereof provided to a plurality of biological particles may be provided to the plurality of biological particles in the same or different manner (e.g., using similar lentiviral vector systems). In some cases, only one type of gRNA molecule or precursor thereof (e.g., a nucleic acid molecule encoding only one type of gRNA molecule) may be provided to a given biological particle (e.g., cell, cell bead, or cell nucleus) of the plurality of biological particles. The plurality of biological particles, or a subset thereof, may have previously been provided with a Cas protein or precursor thereof, and/or may already be configured to express a Cas protein or precursor thereof (e.g., as described herein). Alternatively, a Cas protein (e.g., a Cas protein with which at least a subset of the gRNA molecules of the plurality of gRNA molecules are configured to interact) may be provided to at least a subset of the plurality of biological particles at the same time as the plurality of gRNA molecules or precursors thereof. Subsequent processing of nucleic acid molecules (e.g., chromatin and gRNA molecules or derivatives thereof) of the plurality of biological particles, or a subset thereof, may be performed as described herein.

In an example, a plurality of gRNA molecules or precursors thereof are provided to a plurality of biological particles. The plurality of gRNA molecules or precursors thereof may be provided to the plurality of biological particles such that a given biological particle of the plurality of biological particle is provided with only a single type of gRNA molecule or precursor thereof (e.g., a nucleic acid molecule encoding only a single type of gRNA molecule). The plurality of gRNA molecules or precursors thereof may comprise a plurality of gRNA molecules comprising the same tracrRNA sequence and different crRNA sequences (e.g., as described herein). The plurality of biological particles may comprise cells, cell beads, or cell nuclei from a same source and of a same type. The plurality of biological particles may be configured to express and/or already comprise a Cas protein with which gRNA molecules of the plurality of gRNA molecules are configured to interact. The plurality of gRNA molecules or precursors thereof may be configured to target different genes and/or different regions of one or more genes. Following introduction of the plurality of gRNA molecules or precursors thereof to the plurality of biological particles (e.g., a described herein), the plurality of biological particles may be partitioned among a plurality of partitions (e.g., a plurality of droplets) with a plurality of reagents (e.g., as described herein). A partition of the plurality of partitions may comprise a single biological particle of the plurality of biological particles. The partition may also comprise a bead comprising a plurality of nucleic acid barcode molecules coupled thereto (e.g., as described herein). The partition may also comprise one or more different primer molecules. For example, the partition may comprise a plurality of primer molecules comprising primer molecules configured to hybridize to one or more different crRNA sequences of the plurality of gRNA molecules (or precursors, portions, or derivatives thereof), such that a primer molecule of the plurality of primer molecules may be configured to hybridize to a crRNA sequence of a gRNA molecule provided to the biological particle within the partition. Alternatively or in addition, the partition may comprise primer molecules of a single type, which primer molecules may be configured to hybridize to a tracrRNA sequence or other sequence of a gRNA molecule provided to the biological particle within the partition.

In some instances, the gRNA molecule (or precursor, portion, or derivative thereof) comprises a primer sequence that is complementary to a sequence of the one or more different primer molecules. For example, the gRNA molecule (or precursor, portion, or derivative thereof) may comprise a first primer sequence that is complementary to a sequence of one primer molecule. Alternatively or in addition, the gRNA molecule (or precursor, portion, or derivative thereof) may comprise (i) a first primer sequence that is complementary to a first sequence of a first primer molecule of the one or more different primer molecules and (ii) a second primer sequence that is complementary to a second sequence of a second primer molecule of the one or more different primer molecules. In such instances, the first primer sequence and the second primer sequence may be disposed at opposite ends of the gRNA sequence of the gRNA molecule (or precursor, portion, or derivative thereof). In instances where the gRNA molecule (or precursor, portion, or derivative thereof) comprises a gRNA-identifying barcode sequence, the first primer sequence and the second primer sequence may be disposed at opposite ends of the gRNA sequence, the gRNA-identifying barcode sequence, or both the gRNA sequence and the gRNA-identifying barcode sequence. In some embodiments, a first extension product generated comprises a forward primer sequence (or complement thereof) and a gRNA molecule sequence or a gRNA-identifying barcode sequence (or complement thereof). In some cases, the first extension product further comprises a common gRNA sequence from a region downstream of the target gRNA sequence. In some embodiments, a second extension product generated using the second primer molecule comprises, in the following order: forward primer sequence—gRNA molecule sequence or gRNA-identifying barcode sequence—reverse primer sequence (or a complementary sequence thereof of any of the sequences).

Nucleic acid molecules (e.g., chromatin and a gRNA molecule or derivatives thereof) of the biological particle within the partition may be processed to provide barcoded nucleic acid molecules, which barcoded nucleic acid molecules may be recovered from the partition (e.g., upon pooling of droplets of a plurality of droplets), further processed, and subjected to nucleic acid sequencing. Processing of chromatin and gRNA molecules or derivatives thereof may proceed as described elsewhere herein. Nucleic acid sequencing may be used to identify the gRNA molecule or precursor thereof (e.g., the perturbation) introduced to the biological particle as well as the effect of the perturbation on the biological particle (e.g., via ATAC sequencing). In this manner, a plurality of different perturbations (e.g., gRNA molecules or precursors thereof) may be screened to determine their effects on genomic DNA. Parallel RNA processing may also be performed (e.g., as described herein) to examine gene expression.

Perturbations introduced to a biological particle (e.g., cell, cell bead, or cell nucleus) may be specific (e.g., CRISPR inhibition or other perturbations) and/or unbiased perturbations. For example, unbiased perturbations may be applied to uncover distinct trans-factor activities that may occur during a biological process such as cell differentiation, metastasis, migration, etc. A global analysis of perturbed factors and their corresponding target regions may reveal an inter-connected network of regulation that yields information otherwise not accessible from single-target perturbations.

The methods, systems, and kits provided herein may be used to infer a variety of genotype-phenotype relationships. In some cases. Perturb-ATAC may be applied to transcription factors. Perturb-ATAC may also be applied to, for example, chromatin-modifying factors, and noncoding RNAs Combinations of factors may be assayed using Perturb-ATAC In some cases, Perturb-ATAC may be used to uncover hierarchical organization of TFs that govern cell behavior. For example, cell state, cell variation, cell fate, cell pathology (e g., disease-associated cis-regulatory elements), epistatic relationships of TFs, genomic co-localization of TFs, and/or synergistic and/or inhibitory interactions of TEs may be inferred from Perturb-ATAC. Gene regulatory networks in development and disease may also be analyzed using Perturb-ATAC. In some cases, Perturb-ATAC may uncover epigenetic interactions that establish gene expression patterns that underlie development, differentiation, cell-cell and/or cell-matrix interactions, and cell-environmental responses. In some cases, Perturb-ATAC may be used to identify gene targets, gene signatures, transcription factors, regulatory factors, and/or cell states that are impacted by perturbations to a given cell and/or drive distinct cell states.

The methods provided herein may be used for a variety of applications in biological discovery. For example, the methods provided herein (e.g., Perturb-ATAC) may identify epigenomic functions of chromatin regulators, transcription factors and noncoding RNAs. Performing a Perturb-ATAC screen may be used to compare how broadly-expressed and lineage-specific trans-factors shape the chromatin landscape of a cell type. Perturb-ATAC may also identify epigenomic phenotypes associated with genetic perturbations of diverse categories of trans-factors. For example, as a control experiment, an analysis of aggregate ATAC-seq profiles of cells receiving non-human-genome-targeting barcodes may be expected to result in little to no change in chromatin accessibility; however, a selective perturbation with gRNA targeted to, for example, DNMT3a, may result in changes in the accessibility.

In some cases, more than one perturbation (i.e., application of more than one gRNA) may be applied to cells. Combinations of perturbations, followed by ATAC-seq may reveal how, for example, transcription factors function together to establish chromatin landscape in cells. Perturb-ATAC may also be useful, in non-limiting examples, in identifying co-varying regulatory networks across single cells, measuring the effects of perturbation on one or more regulatory networks, and/or inferring regulatory relationships between perturbed factor and the constituent factors in the regulatory network. In one example, dual perturbations in single cells for a subset of factors followed by Perturb-ATAC may determine the degree of genetic interaction across all genomic features. Perturb-ATAC on cells that with more than one perturbation may be used to characterize trans-factor relationships as “expected” (i.e., based on the combination of the effects of each perturbation alone) or “unexpected” (i.e., non-additive, suggesting interaction between the perturbations). The “unexpected” relationships may be trans-factors that act synergistically, have a canceling effect, or interact in a non-additive way. Epistatic interactions analyzed from more than one perturbation may also be useful in screening for disease-related transcription factors and mapping interactions of epigenomic networks.

In some cases, the occupancy and positioning of nucleosomes genome-wide may be inferred by the fragment sizes obtained from ATAC-seq Assessment of trans factors, which may control accessibility of a locus by regulating the binding of TFs in pre-established nucleosome-free regions and/or by altering the positions or occupancy of local nucleosomes, may yield additional information. In some cases, ATAC-seq data may determine whether changes in ATAC-seq signal at genomic regions are associated with alterations in nucleosome structure rather than exchange of TF binding within a stable nucleosome scaffold.

In some cases, Perturb-ATAC analysis may inform of pathological processes. For example, Perturb-ATAC may inform regulators of noncoding regions that contain genetic variants associated with human disease. Selective perturbation of candidate factors may reveal disease-specific activities of several TFS.

In some instances, the method can be used to compare two samples. A first epigenetic map may be generated by analyzing a first cell or a first population of cells. A second epigenetic map may be generated by analyzing a second cell or a second population of cells. The two epigenetic maps may be compared, consolidated, or otherwise processed against or with each other. For example, the first epigenetic map may be mapped to the second epigenetic map, such as to determine or characterize accessibility of chromatin (e.g., chromatin openness) or transcription factor occupancy, optionally for quality control, optionally in response to perturbation of target genes, and/or changes thereof. In some instances, the first input (first cell or first population of cell) may be a clone of the second input (second cell or second population of cell), or vice versa. In some instances, the first input and the second input may be obtained from a same source at different times. In some instances, the first input and the second input may be obtained from different sources. In some instances, the first input and the second input may be obtained from different locations or regions of the same source (e.g., individual). In some instances, the first input may be a pre-treated input and the second input may be a post-treated input, such as by treatment with an agent (e g., test agent), a drug, a perturbation agent, and the like. In such cases, the first input and the second input may be clones or identical populations, and the second input may be incubated with the treatment before the assays and/or methods described herein are performed. In some instances, these methods can be used to determine the mode of action of a test agent, to identify changes in chromatin structure or transcription factor occupancy in response to the drug, for example. In some instances, one of the two samples may be a control sample.

The method described above may also be used to provide a diagnosis and/or prognosis, such as based on one or more epigenetic maps, such as for a patient.

The method set forth herein may be used to provide a reliable diagnostic to any condition associated with altered chromatin or DNA binding protein occupancy. The method can be applied to the characterization, classification, differentiation, grading, staging, diagnosis, or prognosis of a condition characterized by an epigenetic pattern (e.g., a pattern of chromatin accessibility or DNA binding protein occupancy). For example, the method can be used to determine whether the epigenetic map of a sample from an individual suspected of being affected by a disease or condition is the same or different compared to a sample that is considered “normal” with respect to the disease or condition. In particular embodiments, the method can be directed to diagnosing an individual with a condition that is characterized by an epigenetic pattern at a particular locus in a test sample, where the pattern is correlated with the condition. The methods can also be used for predicting the susceptibility of an individual to a condition.

Exemplary conditions that are suitable for analysis using the methods set forth herein can be, for example, cell proliferative disorder or predisposition to cell proliferative disorder; metabolic malfunction or disorder; immune malfunction, damage or disorder; CNS malfunction, damage or disease; symptoms of aggression or behavioral disturbance; clinical, psychological and social consequences of brain damage; psychotic disturbance and personality disorder; dementia or associated syndrome; cardiovascular disease, malfunction and damage, malfunction, damage or disease of the gastrointestinal tract; malfunction, damage or disease of the respiratory system; lesion, inflammation, infection, immunity and/or convalescence, malfunction, damage or disease of the body as an abnormality in the development process: malfunction, damage or disease of the skin, the muscles, the connective tissue or the bones; endocrine and metabolic malfunction, damage or disease, headache or sexual malfunction, and combinations thereof.

In some instances, the method can provide a prognosis, e.g., to determine if a patient is at risk for recurrence. Cancer recurrence is a concern relating to a variety of types of cancer. The prognostic method can be used to identify surgically treated patients likely to experience cancer recurrence so that they can be offered additional therapeutic options, including preoperative or postoperative adjuncts such as chemotherapy, radiation, biological modifiers and other suitable therapies. The methods are especially effective for determining the risk of metastasis in patients who demonstrate no measurable metastasis at the time of examination or surgery.

The method can also be used to determining a proper course of treatment for a patient having a disease or condition, e.g., a patient that has cancer. A course of treatment refers to the therapeutic measures taken for a patient after diagnosis or after treatment. For example, a determination of the likelihood for recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or adjust the span of time during which the patient is treated.

The present disclosure also provides systems for nucleic acid processing (e.g., parallel processing of DNA and gRNA molecules or a precursors, portions, or a derivatives thereof, any of which may be associated with the gRNA or a gRNA-identifying barcode). A system for nucleic acid processing may comprise a partition (e.g., droplet or well) comprising: (i) a biological particle (e.g., cell, cell bead, or cell nucleus) comprising (1) a deoxyribonucleic acid (DNA) molecule and (2) a guide ribonucleic acid (gRNA) molecule. The partition may further comprise (ii) a plurality of nucleic acid barcode molecules, wherein the plurality of nucleic acid barcode molecules comprises a first nucleic acid barcode molecule and a second nucleic acid barcode molecule, wherein the first nucleic acid barcode molecule and the second nucleic acid barcode molecule comprise a common barcode sequence, and wherein the second nucleic acid barcode molecule comprises an adapter sequence. The partition may further comprise (iii) a plurality of primer molecules, wherein the plurality of primer molecules comprises a first primer molecule and a second primer molecule, wherein the first primer molecule comprises a sequence complementary to the adapter sequence. The system may comprise one or more computer processors that are individually or collectively programmed to subject the partition and its contents to conditions sufficient for (i) the DNA molecule or a derivative thereof and the first nucleic acid barcode molecule to generate a first barcoded nucleic acid molecule and (ii) the gRNA molecule or a derivative thereof, the second nucleic acid barcode molecule, the first primer molecule, and the second primer molecule to generate a second barcoded nucleic acid molecule. The first barcoded nucleic acid molecule may comprise the common barcode sequence, or a complement thereof. The second barcoded nucleic acid molecule may also comprise the common barcode sequence, or a complement thereof. The one or more computer processors may be further individually or collectively programmed to recover the first barcoded nucleic acid molecule or a derivative thereof and the second barcoded nucleic acid molecule or a derivative thereof from the partition and optionally subject the barcoded nucleic acid molecules or derivatives thereof to additional processing (e.g., as described herein).

In another example, a system for nucleic acid processing may comprise a partition (e.g., droplet or well) comprising: (i) a biological particle (e.g., cell, cell bead, or cell nucleus) comprising (1) a deoxyribonucleic acid (DNA) molecule and (2) a guide ribonucleic acid (gRNA) molecule. The partition may also comprise (ii) a first nucleic acid barcode molecule and a second nucleic acid barcode molecule, wherein the first nucleic acid barcode molecule and the second nucleic acid barcode molecule comprise a common barcode sequence. The system may comprise one or more computer processors that are individually or collectively programmed to subject the partition and its contents to conditions sufficient for (i) the DNA molecule or a derivative thereof and the first nucleic acid barcode molecule to generate a first barcoded nucleic acid molecule and (ii) the gRNA molecule or a derivative thereof and the second nucleic acid barcode molecule to generate a second barcoded nucleic acid molecule. The first barcoded nucleic acid molecule may comprise the common barcode sequence, or a complement thereof. The second barcoded nucleic acid molecule may comprise the common barcode sequence, or a complement thereof. The one or more computer processors may be further individually or collectively programmed to recover the first barcoded nucleic acid molecule or a derivative thereof and the second barcoded nucleic acid molecule or a derivative thereof from the partition and optionally subject the barcoded nucleic acid molecules or derivatives thereof to additional processing (e.g., as described herein).

The present disclosure also provides kits for nucleic acid processing. A kit for nucleic acid processing may comprise a plurality of gRNA molecules or precursors thereof. The plurality of gRNA molecules or precursors thereof may comprise a plurality of viral vectors encoding a plurality of gRNA molecules (e.g., as described herein). The plurality of viral vectors (e.g., lentiviral vectors) may further encode one or more barcode sequences, selection proteins (e.g., fluorescent proteins), and promoters (e.g., U6 promoters). The plurality of gRNA molecules may have the same sequence (e.g., target the same region of the same gene). Alternatively, the plurality of gRNA molecules or precursors thereof may be configured to target a plurality of different genes and/or a plurality of different regions of a same gene (e.g., the plurality of gRNA molecules or derivatives thereof may comprise a plurality of different crRNA sequences). The kit may further comprise a plurality of primer molecules configured to interact with the plurality of gRNA molecules (or precursors, portions, or derivatives thereof). For example, the plurality of primer molecules may comprise a first set of primer molecules configured to interact with (e.g., hybridize to) a first set of gRNA molecules (or precursor, portion, or derivative thereof) and a second set of primer molecules configured to interact with a second set of gRNA molecules (or precursor, portion, or derivative thereof). The plurality of primer molecules may comprise primer sequences configured to interact with crRNA sequences of the gRNA molecules (or precursor, portion, or derivative thereof) and/or primer sequences configured to interact with tracrRNA sequences of the gRNA molecules (or precursor, portion, or derivative thereof). Alternatively or in addition, the plurality of primer molecules may comprise primer sequences configured to interact with other sequences of the gRNA molecules (or precursor, portion, or derivative thereof). As described herein, in some instances, the gRNA molecule (or precursor, portion, or derivative thereof) comprises a primer sequence that is complementary to a sequence of the plurality of primer molecules. For example, the gRNA molecule (or precursor, portion, or derivative thereof) may comprise a first primer sequence that is complementary to a sequence of one primer molecule. Alternatively or in addition, the gRNA molecule (or precursor, portion, or derivative thereof) may comprise (i) a first primer sequence that is complementary to a first sequence of a first primer molecule of the plurality of primer molecules and (ii) a second primer sequence that is complementary to a second sequence of a second primer molecule of the plurality of primer molecules. In such instances, the first primer sequence and the second primer sequence may be disposed at opposite ends of the gRNA sequence of the gRNA molecule (or precursor, portion, or derivative thereof). In instances where the gRNA molecule (or precursor, portion, or derivative thereof) comprises a gRNA-identifying barcode sequence, the first primer sequence and the second primer sequence may be disposed at opposite ends of the gRNA sequence, the gRNA-identifying barcode sequence, or both the gRNA sequence and the gRNA-identifying barcode sequence. The plurality of primer molecules may be coupled to a support (e.g., a bead). Alternatively, the plurality of primer molecules may not be coupled to a support (e.g., a bead). The kit may comprise instructions for using the other components therein (e.g., according to the methods provided herein). The kit may also include materials for processing chromatin, such as transposases and associated materials (e.g., as described herein).

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Application No. PCT/US20/17785, which is entirely incorporated herein by reference for all purposes.

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particle methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as analyte carriers or analyte carriers, in some cases, individual analyte carriers such as single cells. In some examples, reagents may be encapsulated and/or partitioned (e.g., co-partitioned with analyte carriers) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

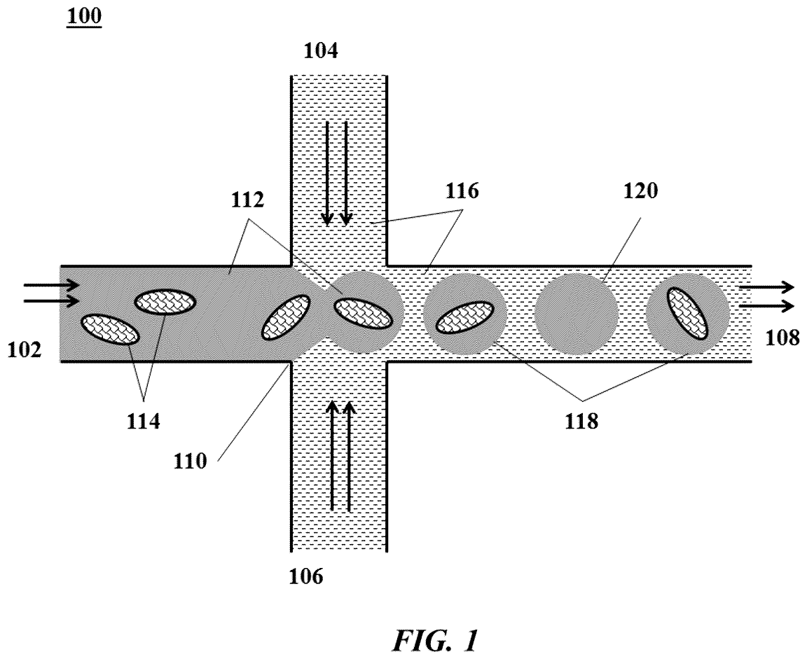

In the case of droplets in an emulsion, allocating individual particles to discrete partitions may in one non-limiting example be accomplished by introducing a flowing stream of particles in an aqueous fluid into a flowing stream or reservoir of a non-aqueous fluid, such that droplets are generated (see generally, e.g., FIGS. 1-7B). Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

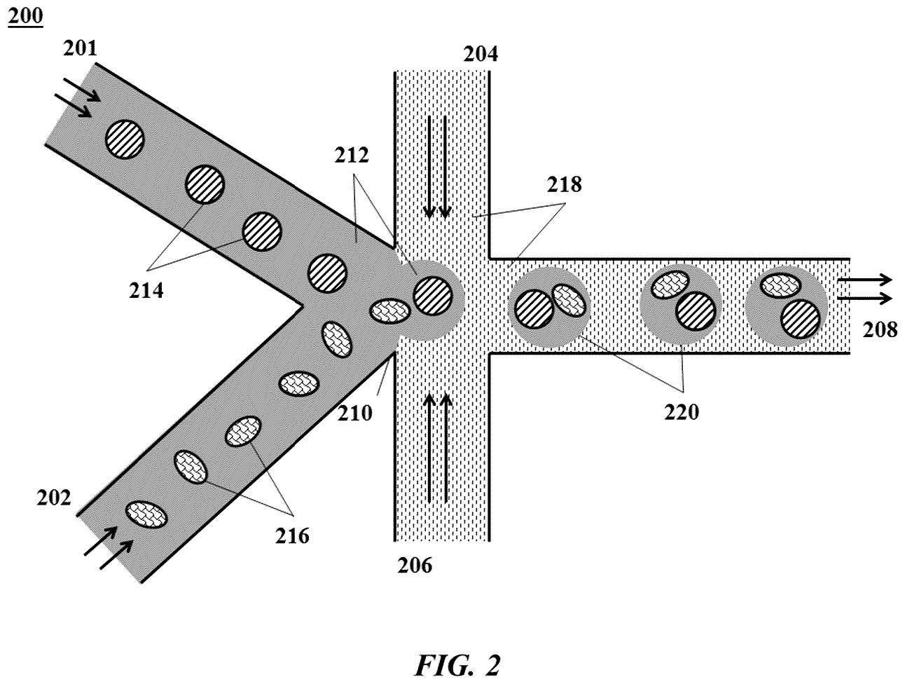

As will be appreciated, the above-described occupancy rates are also applicable to partitions that include both biological particles and additional reagents, including, but not limited to, microcapsules or beads (e.g., gel beads) carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca²⁺ ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.”

A cell bead can contain biological particles (e.g., a cell) or macromolecular constituents (e.g., RNA, DNA, proteins, etc.) of biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents). In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

### Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a microcapsule, droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a microcapsule or bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the microcapsule or bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

A well may comprise free reagents and/or reagents encapsulated in, or otherwise coupled to or associated with, microcapsules, beads, or droplets. Any of the reagents described in this disclosure may be encapsulated in, or otherwise coupled to, a microcapsule, droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, IRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well may be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a microcapsule, bead, or droplet that comprises a set of reagents that has a similar attribute (e.g., a set of enzymes, a set of minerals, a set of oligonucleotides, a mixture of different barcode molecules, a mixture of identical barcode molecules). In other cases, a microcapsule, bead, or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different microcapsule, droplet, or bead, or within a solution within a partition (e.g., microwell) of the system.

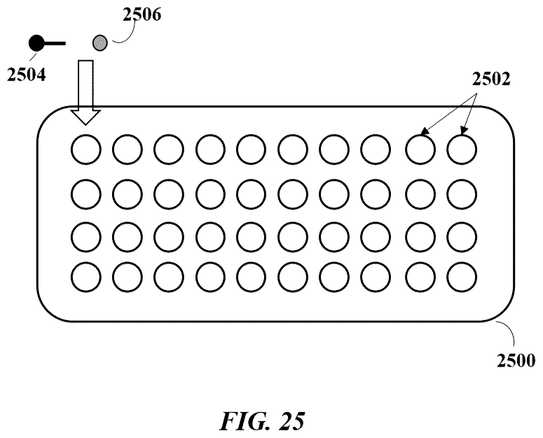

FIG. 25 schematically illustrates an example of a microwell array. The array can be contained within a substrate 2500. The substrate 2500 comprises a plurality of wells 2502. The wells 2502 may be of any size or shape, and the spacing between the wells, the number of wells per substrate, as well as the density of the wells on the substrate 2500 can be modified, depending on the particular application. In one such example application, a sample molecule 2506, which may comprise a cell or cellular components (e.g., nucleic acid molecules) is co-partitioned with a bead 2504, which may comprise a nucleic acid barcode molecule coupled thereto. The wells 2502 may be loaded using gravity or other loading technique (e.g., centrifugation, liquid handler, acoustic loading, optoelectronic, etc.). In some instances, at least one of the wells 2502 contains a single sample molecule 2506 (e.g., cell) and a single bead 2504.

Reagents may be loaded into a well either sequentially or concurrently. In some cases, reagents are introduced to the device either before or after a particular operation. In some cases, reagents (which may be provided, in certain instances, in microcapsules, droplets, or beads) are introduced sequentially such that different reactions or operations occur at different steps. The reagents (or microcapsules, droplets, or beads) may also be loaded at operations interspersed with a reaction or operation step. For example, microcapsules (or droplets or beads) comprising reagents for fragmenting polynucleotides (e.g., restriction enzymes) and/or other enzymes (e.g., transposases, ligases, polymerases, etc.) may be loaded into the well or plurality of wells, followed by loading of microcapsules, droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a microcapsule, bead, or droplet. These microcapsules, beads, or droplets may be loaded into a partition (e.g., a microwell) before, after, or concurrently with the loading of a cell, such that each cell is contacted with a different microcapsule, bead, or droplet. This technique may be used to attach a unique nucleic acid barcode molecule to nucleic acid molecules obtained from each cell. Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or microcapsule may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to: dynamic live-cell tracking, cell-cell interactions (when two or more cells are co-partitioned), cell proliferation, etc. Alternatively or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

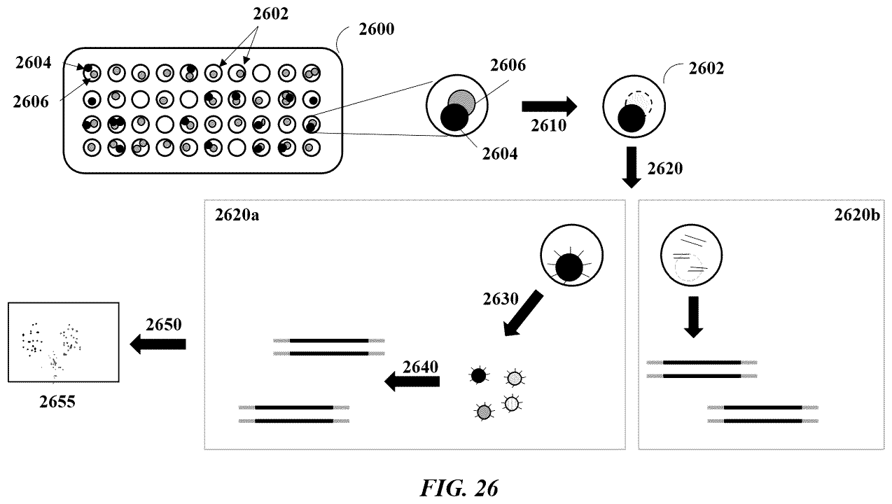

FIG. 26 schematically shows an example workflow for processing nucleic acid molecules within a sample. A substrate 2600 comprising a plurality of microwells 2602 may be provided. A sample 2606 which may comprise a cell, cell bead, cellular components or analytes (e.g., proteins and/or nucleic acid molecules) can be co-partitioned, in a plurality of microwells 2602, with a plurality of beads 2604 comprising nucleic acid barcode molecules. During process 2610, the sample 2606 may be processed within the partition. For instance, in the case of live cells, the cell may be subjected to conditions sufficient to lyse the cells and release the analytes contained therein. In process 2620, the bead 2604 may be further processed. By way of example, processes 2620a and 2620b schematically illustrate different workflows, depending on the properties of the bead 2604.

In 2620a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 2630, the beads 2604 from multiple wells 2602 may be collected and pooled. Further processing may be performed in process 2640. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 2650, further characterization, such as sequencing may be performed to generate sequencing reads. The sequencing reads may yield information on individual cells or populations of cells, which may be represented visually or graphically, e.g., in a plot 2655.

In 2620b, the bead comprises nucleic acid barcode molecules that are releasably attached thereto, as described below. The bead may degrade or otherwise release the nucleic acid barcode molecules into the well 2602; the nucleic acid barcode molecules may then be used to barcode nucleic acid molecules within the well 2602. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 2650, further characterization, such as sequencing may be performed to generate sequencing reads. The sequencing reads may yield information on individual cells or populations of cells, which may be represented visually or graphically, e.g., in a plot 2655.

### Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

A nucleic acid barcode molecule may contain a barcode sequence and a functional sequence, such as a nucleic acid primer sequence or a template switch oligonucleotide (TSO) sequence.

The solid support may be a bead. A solid support, e.g., a bead, may be porous, non-porous, hollow (e.g., a microcapsule), solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application No. PCT/US20/17785, each of which is herein entirely incorporated by reference for all purposes.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles 216 along the channel segment 202 into junction 210. The plurality of biological particles 216 may be sourced from a suspension of biological particles. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Beads, biological particles and droplets may flow along channels. In some examples, beads, analyte carriers, and droplets may flow along channels (e.g., the channels of a microfluidic device), in some cases at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual biological particle 216. A discrete droplet that is generated may include a barcode or other reagent carrying bead 214. A discrete droplet generated may include both an individual biological particle and a barcode carrying bead, such as droplets 220. In some instances, a discrete droplet may include more than one individual biological particle or no biological particle. In some instances, a discrete droplet may include more than one bead or no bead. A discrete droplet may be unoccupied (e.g., no beads, no biological particles).

Beneficially, a discrete droplet partitioning a biological particle and a barcode carrying bead may effectively allow the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide) that comprises one or more functional sequences, such as a TSO sequence or a primer sequence (e.g., a poly T sequence, or a nucleic acid primer sequence complementary to a target nucleic acid sequence and/or for amplifying a target nucleic acid sequence, a random primer, or a primer sequence for messenger RNA) that is useful for incorporation into the bead, etc.) and/or one or more barcode sequences. The one or more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence (or a portion thereof) for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina sequencing. In some cases, the nucleic acid molecule can comprise a barcode sequence. In some cases, the nucleic acid molecule can further comprise a unique molecular identifier (UMI). In some cases, the nucleic acid molecule can comprise an R1 primer sequence for Illumina sequencing. In some cases, the nucleic acid molecule can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

In some cases, the nucleic acid molecule can comprise one or more functional sequences. For example, a functional sequence can comprise a sequence for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the functional sequence can comprise a barcode sequence or multiple barcode sequences. In some cases, the functional sequence can comprise a unique molecular identifier (UMI). In some cases, the functional sequence can comprise a primer sequence (e.g., an R1 primer sequence for Illumina sequencing, an R2 primer sequence for Illumina sequencing, etc.). In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

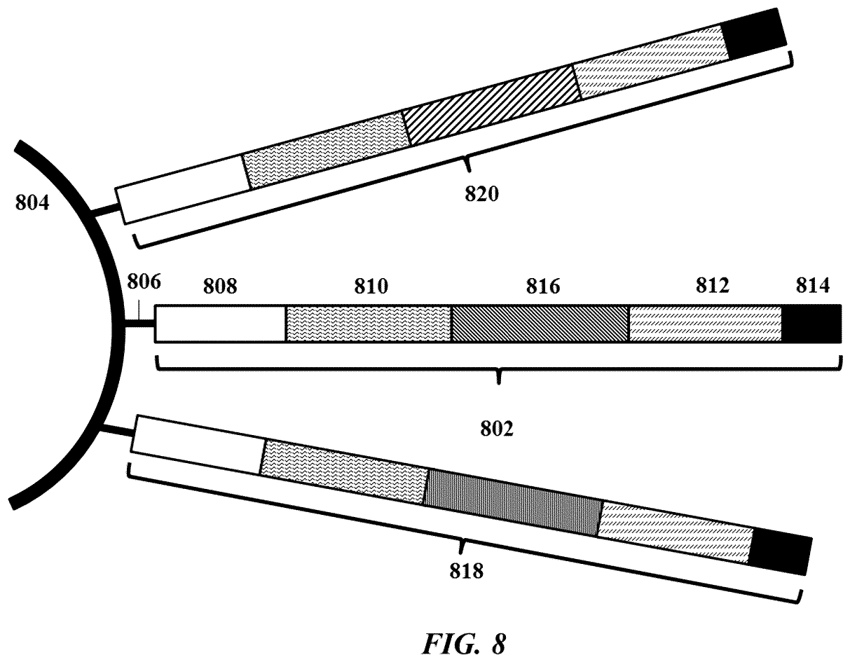

FIG. 8 illustrates an example of a barcode carrying bead. A nucleic acid molecule 802, such as an oligonucleotide, can be coupled to a bead 804 by a releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid molecules (e.g., including nucleic acid molecule 802) coupled to the same bead 804. Alternatively or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

In operation, a biological particle (e.g., cell, DNA, RNA, etc.) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the released nucleic acid molecules (e.g., 802) can hybridize to the poly-A tail of a mRNA molecule. Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

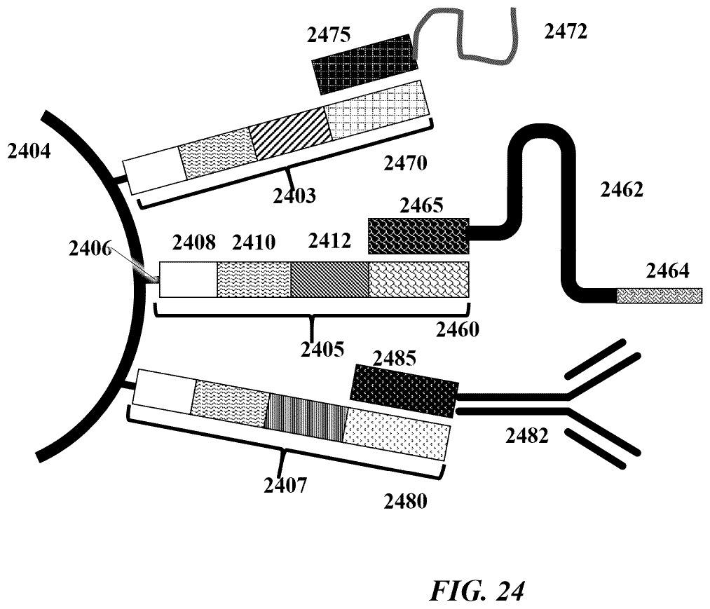

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, a biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules. FIG. 24 illustrates another example of a barcode carrying bead. A nucleic acid molecule 2405, such as an oligonucleotide, can be coupled to a bead 2404 by a releasable linkage 2406, such as, for example, a disulfide linker. The nucleic acid molecule 2405 may comprise a first capture sequence 2460. The same bead 2404 may be coupled (e.g., via releasable linkage) to one or more other nucleic acid molecules 2403, 2407 comprising other capture sequences. The nucleic acid molecule 2405 may be or comprise a barcode. As noted elsewhere herein, the structure of the barcode may comprise a number of sequence elements, such as a functional sequence 2408 (e.g., flow cell attachment sequence, sequencing primer sequence, etc.), a barcode sequence 2410 (e.g., bead-specific sequence common to bead, partition-specific sequence common to partition, etc.), and a unique molecular identifier 2412 (e.g., unique sequence within different molecules attached to the bead), or partial sequences thereof. The capture sequence 2460 may be configured to attach to a corresponding capture sequence 2465. In some instances, the corresponding capture sequence 2465 may be coupled to another molecule that may be an analyte or an intermediary carrier. For example, as illustrated in FIG. 24, the corresponding capture sequence 2465 is coupled to a guide RNA molecule 2462 comprising a target sequence 2464, wherein the target sequence 2464 is configured to attach to the analyte. Another oligonucleotide molecule 2407 attached to the bead 2404 comprises a second capture sequence 2480 which is configured to attach to a second corresponding capture sequence 2485. As illustrated in FIG. 24, the second corresponding capture sequence 2485 is coupled to an antibody 2482. In some cases, the antibody 2482 may have binding specificity to an analyte (e.g., surface protein). Alternatively, the antibody 2482 may not have binding specificity. Another oligonucleotide molecule 2403 attached to the bead 2404 comprises a third capture sequence 2470 which is configured to attach to a second corresponding capture sequence 2475. As illustrated in FIG. 24, the third corresponding capture sequence 2475 is coupled to a molecule 2472. The molecule 2472 may or may not be configured to target an analyte. The other oligonucleotide molecules 2403, 2407 may comprise the other sequences (e.g., functional sequence, barcode sequence, UMI, etc.) described with respect to oligonucleotide molecule 2405. While a single oligonucleotide molecule comprising each capture sequence is illustrated in FIG. 24, it will be appreciated that, for each capture sequence, the bead may comprise a set of one or more oligonucleotide molecules each comprising the capture sequence. For example, the bead may comprise any number of sets of one or more different capture sequences. Alternatively or in addition, the bead 2404 may comprise other capture sequences. Alternatively or in addition, the bead 2404 may comprise fewer types of capture sequences (e.g., two capture sequences). Alternatively or in addition, the bead 2404 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may comprise pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include ß-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), ß-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

In some examples, a partition of the plurality of partitions may comprise a single biological particle or analyte carrier (e.g., a single cell or a single nucleus of a cell). In some examples, a partition of the plurality of partitions may comprise multiple biological particles or analyte carriers. Such partitions may be referred to as multiply occupied partitions, and may comprise, for example, two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

### Multiplexing

The present disclosures provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize analyte carriers and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. 20190177800; and U.S. Pat. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In some aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more analyte carriers may be used to characterize an analyte carrier as belonging to a particular set of analyte carriers. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling an analyte carrier may comprise subjecting the antibody-linked barcode molecule or the epitope binding fragment-linked barcode molecule to conditions suitable for binding the antibody to a molecule present on a surface of the analyte carrier. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM. 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM. 7 pM, 6 pM, 5 pM, 4 pM, 3 pM, 2 pM, or 1 pM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into an analyte carrier. Labeling analyte carriers may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in embodiments herein include penetratin, transportan, plsl, TAT(48-60), pVEC, MTS, and MAP Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the analyte carrier. In some instances, fluorophores can interact strongly with lipid bilayers and labeling analyte carriers may comprise subjecting the fluorophore-linked barcode molecule to conditions such that the fluorophore binds to or is inserted into a membrane of the analyte carrier. In some cases, the fluorophore is a water-soluble, organic fluorophore. In some instances, the fluorophore is Alexa 532 maleimide, tetramethylrhodamine-5-malcimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 63SP azide, Atto 647N maleimide. Atto 647 SE, or Sulfo-CyS maleimide. See, e.g., Hughes L D. et al. PLoS One. 2014 Feb. 4, 9(2). e87649, which is bereby incorporated by reference in its entirety for all purposes, for a description of organic fluorophores.

A reporter oligonucleotide may be coupled to a lipophilic molecule, and labeling analyte carriers may comprise delivering the nucleic acid barcode molecule to a membrane of the analyte carrier or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and analyte carrier may be such that the analyte carrier retains the lipophilic molecule (e g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. 20190323088, which is hereby entirely incorporated by reference for all purposes.

As described elsewhere herein, libraries of labelling agents may be associated with a particular cell feature as well as be used to identify analytes as originating from a particular analyte carrier, population, or sample. The analyte carriers may be incubated with a plurality of libraries and a given analyte carrier may comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2): 708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide describes exemplary labelling agents (2110, 2120, 2130) comprising reporter oligonucleotides (2140) attached thereto. Labelling agent 2110 (e.g., any of the labelling agents described herein) is attached (either directly, e.g., covalently attached, or indirectly) to reporter oligonucleotide 2140. Reporter oligonucleotide 2140 may comprise barcode sequence 2142 that identifies labelling agent 2110. Reporter oligonucleotide 2140 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

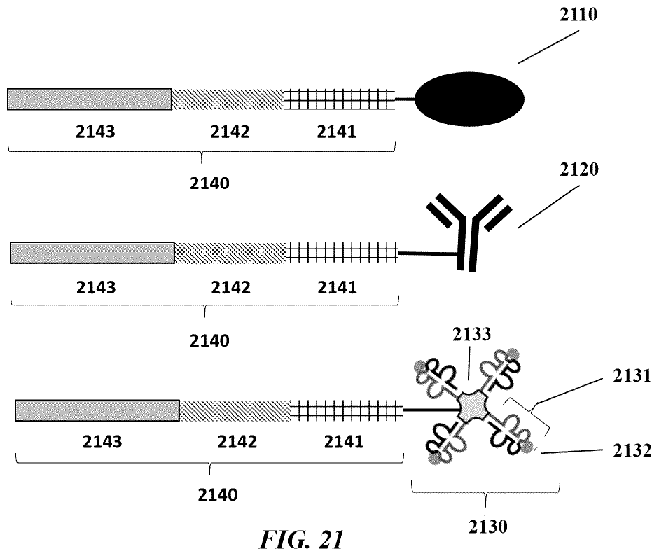

Referring to FIG. 21, in some instances, reporter oligonucleotide 2140 conjugated to a labelling agent (e.g., 2110, 2120, 2130) comprises a primer sequence 2141, a barcode sequence that identifies the labelling agent (e.g., 2110, 2120, 2130), and functional sequence 2143. Functional sequence 2143 may be configured to hybridize to a complementary sequence, such as a complementary sequence present on a nucleic acid barcode molecule 2190 (not shown), such as those described elsewhere herein. In some instances, nucleic acid barcode molecule 2190 is attached to a support (e.g., a bead, such as a gel bead), such as those described elsewhere herein. For example, nucleic acid barcode molecule 2190 may be attached to the support via a releasable linkage (e.g., comprising a labile bond), such as those described elsewhere herein. In some instances, reporter oligonucleotide 2140 comprises one or more additional functional sequences, such as those described above.

In some instances, the labelling agent 2110 is a protein or polypeptide (e.g., an antigen or prospective antigen) comprising reporter oligonucleotide 2140. Reporter oligonucleotide 2140 comprises barcode sequence 2142 that identifies polypeptide 2110 and can be used to infer the presence of an analyte, e.g., a binding partner of polypeptide 2110 (i.e., a molecule or compound to which polypeptide 2110 can bind). In some instances, the labelling agent 2110 is a lipophilic moiety (e.g., cholesterol) comprising reporter oligonucleotide 2140, where the lipophilic moiety is selected such that labelling agent 2110 integrates into a membrane of a cell or nucleus. Reporter oligonucleotide 2140 comprises barcode sequence 2142 that identifies lipophilic moiety 2110 which in some instances is used to tag cells (e.g., groups of cells, cell samples, etc.) and may be used for multiplex analyses as described elsewhere herein. In some instances, the labelling agent is an antibody 2120 (or an epitope binding fragment thereof) comprising reporter oligonucleotide 2140. Reporter oligonucleotide 2140 comprises barcode sequence 2142 that identifies antibody 2120 and can be used to infer the presence of, e.g., a target of antibody 2120 (i.e., a molecule or compound to which antibody 2120 binds). In other embodiments, labelling agent 2130 comprises an MHC molecule 2131 comprising peptide 2132 and reporter oligonucleotide 2140 that identifies peptide 2132. In some instances, the MHC molecule is coupled to a support 2133. In some instances, support 2133 may be a polypeptide, such as streptavidin, or a polysaccharide, such as dextran. In some instances, reporter oligonucleotide 2140 may be directly or indirectly coupled to MHC labelling agent 2130 in any suitable manner. For example, reporter oligonucleotide 2140 may be coupled to MHC molecule 2131, support 2133, or peptide 2132. In some embodiments, labelling agent 2130 comprises a plurality of MHC molecules, (e.g. is an MHC multimer, which may be coupled to a support (e.g., 2133)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. 20190367969, each of which is herein entirely incorporated by reference for all purposes.

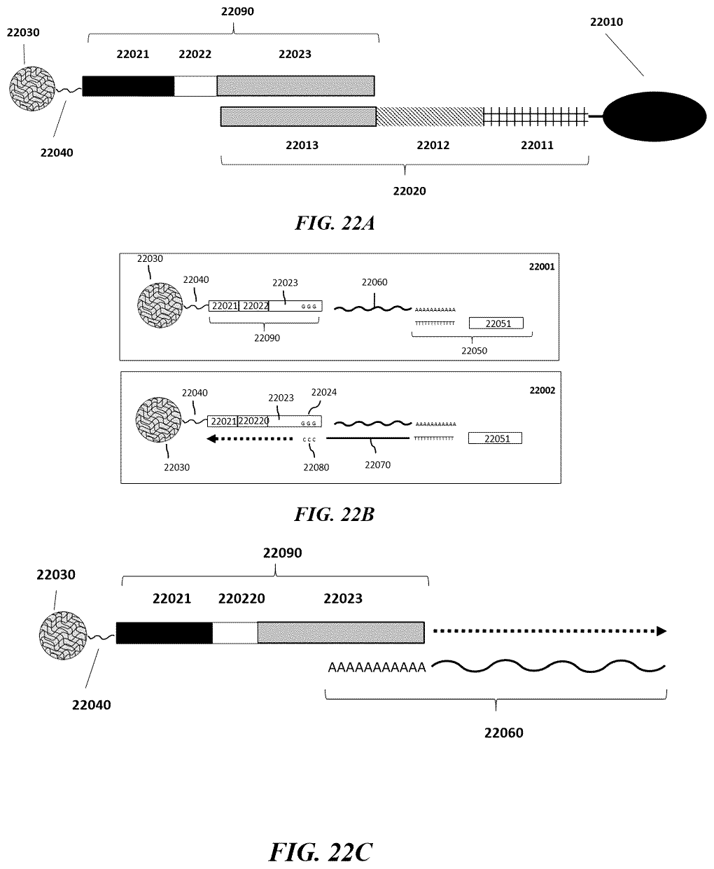

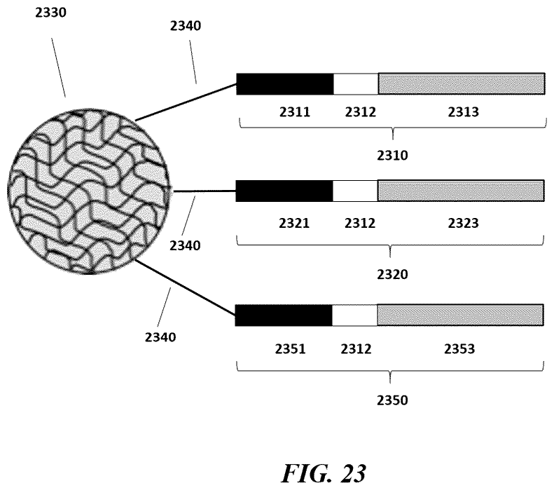

FIG. 23 illustrates another example of a barcode carrying bead. In some embodiments, analysis of multiple analytes (e.g., RNA and one or more analytes using labelling agents described herein) may comprise nucleic acid barcode molecules as generally depicted in FIG. 23. In some embodiments, nucleic acid barcode molecules 2310 and 2320 are attached to support 2330 via a releasable linkage 2340 (e.g., comprising a labile bond) as described elsewhere herein. Nucleic acid barcode molecule 2310 may comprise adapter sequence 2311, barcode sequence 2312 and adapter sequence 2313. Nucleic acid barcode molecule 2320 may comprise adapter sequence 2321, barcode sequence 2312, and adapter sequence 2323, wherein adapter sequence 2323 comprises a different sequence than adapter sequence 2313. In some instances, adapter 2311 and adapter 2321 comprise the same sequence. In some instances, adapter 2311 and adapter 2321 comprise different sequences. Although support 2330 is shown comprising nucleic acid barcode molecules 2310 and 2320, any suitable number of barcode molecules comprising common barcode sequence 2312 are contemplated herein. For example, in some embodiments, support 2330 further comprises nucleic acid barcode molecule 2350. Nucleic acid barcode molecule 2350 may comprise adapter sequence 2351, barcode sequence 2312 and adapter sequence 2353, wherein adapter sequence 2353 comprises a different sequence than adapter sequence 2313 and 2323. In some instances, nucleic acid barcode molecules (e.g., 2310, 2320, 2350) comprise one or more additional functional sequences, such as a UMI or other sequences described herein. The nucleic acid barcode molecules 2310, 2320 or 2350 may interact with analytes as described elsewhere herein, for example, as depicted in FIGS. 22A-C.

Referring to FIG. 22A, in an instance where cells are labelled with labeling agents, sequence 2223 may be complementary to an adapter sequence of a reporter oligonucleotide. Cells may be contacted with one or more reporter oligonucleotide 2220 conjugated labelling agents 2210 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 2210 which is conjugated to oligonucleotide 2220 and support 2230 (e.g., a bead, such as a gel bead) comprising nucleic acid barcode molecule 2290 is partitioned into a partition amongst a plurality of partitions (e.g., a droplet of a droplet emulsion or a well of a microwell array). In some instances, the partition comprises at most a single cell bound to labelling agent 2210. In some instances, reporter oligonucleotide 2220 conjugated to labelling agent 2210 (e.g., polypeptide, an antibody, pMHC molecule such as an MHC multimer, etc.) comprises a first adapter sequence 2211 (e.g., a primer sequence), a barcode sequence 2212 that identifies the labelling agent 2210 (e.g., the polypeptide, antibody, or peptide of a pMHC molecule or complex), and an adapter sequence 2213. Adapter sequence 2213 may be configured to hybridize to a complementary sequence, such as sequence 2223 present on a nucleic acid barcode molecule 2290. In some instances, oligonucleotide 2220 comprises one or more additional functional sequences, such as those described elsewhere herein.

Barcoded nucleic may be generated (e.g., via a nucleic acid reaction, such as nucleic acid extension or ligation) from the constructs described in FIGS. 22A-C. For example, sequence 22013 may then be hybridized to complementary sequence 22023 to generate (e.g., via a nucleic acid reaction, such as nucleic acid extension or ligation) a barcoded nucleic acid molecule comprising cell (e.g., partition specific) barcode sequence 22022 (or a reverse complement thereof) and reporter barcode sequence 22012 (or a reverse complement thereof). Barcoded nucleic acid molecules can then be optionally processed as described elsewhere herein, e.g., to amplify the molecules and/or append sequencing platform specific sequences to the fragments. See, e.g., U.S. Pat. Pub. No. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 22A-C, or a combination of workflows for an individual analyte, as described elsewhere herein. For example, by using a combination of the workflows as generally depicted in FIGS. 22A-C, multiple analytes can be analyzed.

In some instances, analysis of an analyte (e.g. a nucleic acid, a polypeptides, a carbohydrate, a lipid, etc.) comprises a workflow as generally depicted in FIG. 22A. A nucleic acid barcode molecule 22090 may be co-partitioned with the one or more analytes. In some instances, nucleic acid barcode molecule 22090 is attached to a support 22030 (e.g., a bead, such as a gel bead), such as those described elsewhere herein. For example, nucleic acid barcode molecule 22090 may be attached to support 22030 via a releasable linkage 22040 (e.g., comprising a labile bond), such as those described elsewhere herein. Nucleic acid barcode molecule 22090 may comprise a barcode sequence 22021 and optionally comprise other additional sequences, for example, a UMI sequence 22022 (or other functional sequences described elsewhere herein). The nucleic acid barcode molecule 22090 may comprise a sequence 22023 that may be complementary to another nucleic acid sequence, such that it may hybridize to a particular sequence.

For example, sequence 22023 may comprise a poly-T sequence and may be used to hybridize to mRNA. Referring to FIG. 22C, in some embodiments, nucleic acid barcode molecule 22090 comprises sequence 22023 complementary to a sequence of RNA molecule 22060 from a cell. In some instances, sequence 22023 comprises a sequence specific for an RNA molecule. Sequence 22023 may comprise a known or targeted sequence or a random sequence. In some instances, a nucleic acid extension reaction may be performed, thereby generating a barcoded nucleic acid product comprising sequence 22023, the barcode sequence 22021, UMI sequence 220220, any other functional sequence, and a sequence corresponding to the RNA molecule 22060. In another example, sequence 22023 may be complementary to an overhang sequence or an adapter sequence that has been appended to an analyte. For example, referring to FIG. 22B, panel 22001, in some embodiments, primer 22050 comprises a sequence complementary to a sequence of nucleic acid molecule 22060 (such as an RNA encoding for a BCR sequence) from an analyte carrier. In some instances, primer 22050 comprises one or more sequences 22051 that are not complementary to RNA molecule 22060. Sequence 22051 may be a functional sequence as described elsewhere herein, for example, an adapter sequence, a sequencing primer sequence, or a sequence the facilitates coupling to a flow cell of a sequencer. In some instances, primer 22050 comprises a poly-T sequence. In some instances, primer 22050 comprises a sequence complementary to a target sequence in an RNA molecule. In some instances, primer 22050 comprises a sequence complementary to a region of an immune molecule, such as the constant region of a TCR or BCR sequence. Primer 22050 is hybridized to nucleic acid molecule 22060 and complementary molecule 22070 is generated (see Panel 22002). For example, complementary molecule 22070 may be cDNA generated in a reverse transcription reaction. In some instances, an additional sequence may be appended to complementary molecule 22070. For example, the reverse transcriptase enzyme may be selected such that several non-templated bases 22080 (e.g., a poly-C sequence) are appended to the cDNA. In another example, a terminal transferase may also be used to append the additional sequence. Nucleic acid barcode molecule 22090 comprises a sequence 22024 complementary to the non-templated bases, and the reverse transcriptase performs a template switching reaction onto nucleic acid barcode molecule 22090 to generate a barcoded nucleic acid molecule comprising cell (e.g., partition specific) barcode sequence 22022 (or a reverse complement thereof) and a sequence of complementary molecule 22070 (or a portion thereof). In some instances, sequence 22023 comprises a sequence complementary to a region of an immune molecule, such as the constant region of a TCR or BCR sequence. Sequence 22023 is hybridized to nucleic acid molecule 22060 and a complementary molecule 22070 is generated. For example complementary molecule 22070 may be generated in a reverse transcription reaction generating a barcoded nucleic acid molecule comprising cell (e.g., partition specific) barcode sequence 22022 (or a reverse complement thereof) and a sequence of complementary molecule 22070 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, US Patent Publication No 2015/0376609, filed Jun. 26, 2015, and U.S. Patent Publication No. 2019/0367969, each of which applications is herein entirely incorporated by reference for all purposes

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

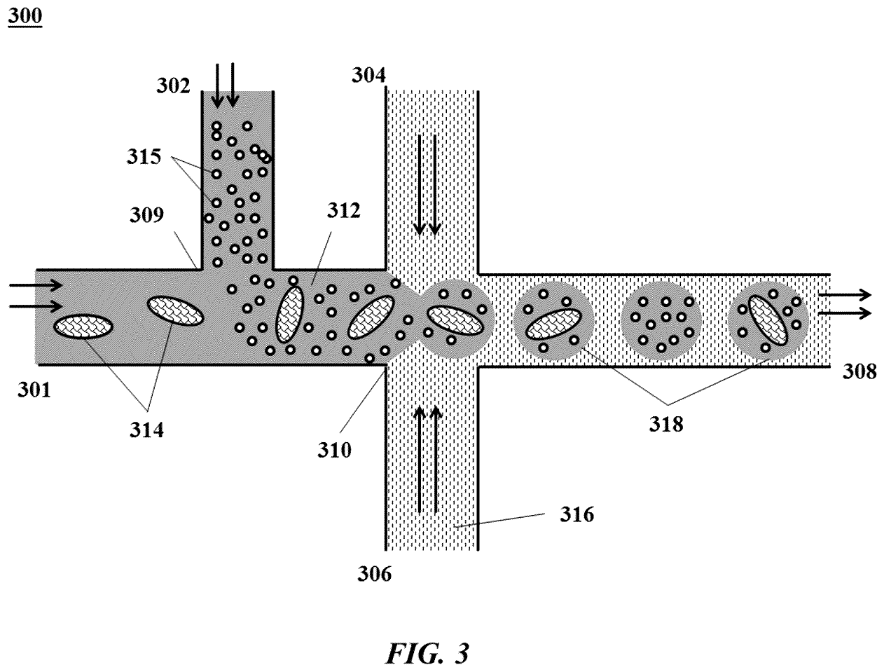

FIG. 3 shows an example of a microfluidic channel structure 300 for co-partitioning biological particles and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of biological particles 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual biological particle 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no biological particles).

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the analyte carriers (e.g., biological particles) described above, other reagents can also be co-partitioned with the analyte carriers (e.g., biological particles), including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated analyte carriers (e.g., a cell or a nucleus in a polymer matrix), the analyte carriers may be exposed to an appropriate stimulus to release the analyte carriers or their contents from a co-partitioned microcapsule. For example, in some cases, a chemical stimulus may be co-partitioned along with an encapsulated analyte carrier (e.g., biological particle) to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative examples, this may be a different and non-overlapping stimulus, in order to allow an encapsulated analyte carrier (e.g., biological particle) to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxy Inosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

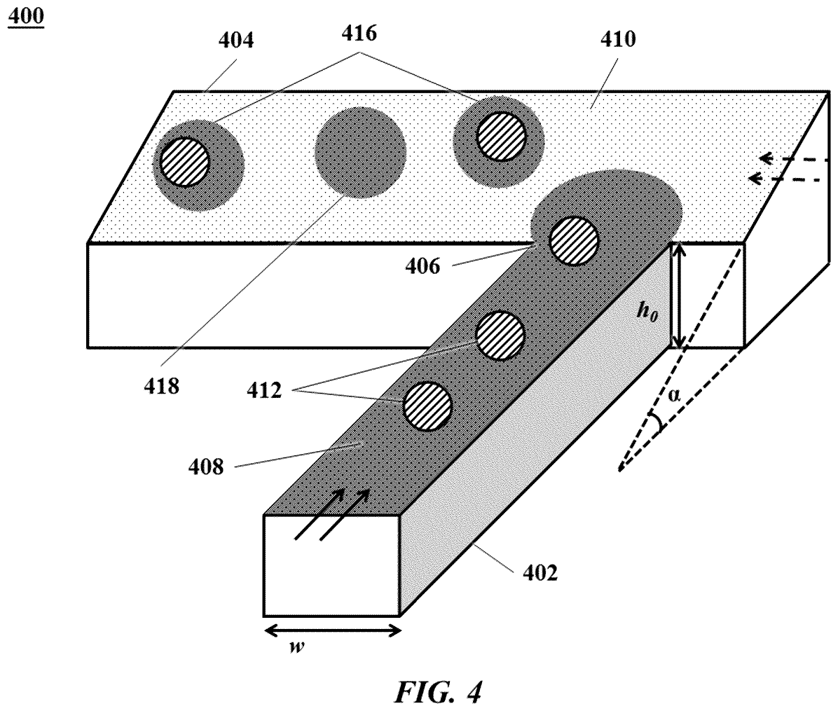

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w′, h₀, a, etc.) of the channel structure 400. A plurality of droplets can be collected in the reservoir 404 by continuously injecting the aqueous fluid 408 from the channel segment 402 through the junction 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more biological particles, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise biological particles (e.g., described with reference to FIGS. 1 and 2). In some instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 may be controlled by controlling the frequency in which the biological particles are introduced into the channel segment 402 and/or the relative flow rates of the fluids in the channel segment 402 and the separate channel. In some instances, the biological particles can be introduced into the channel segment 402 from a plurality of different channels, and the frequency controlled accordingly. In some instances, a first separate channel can introduce beads and a second separate channel can introduce biological particles into the channel segment 402. The first separate channel introducing the beads may be upstream or downstream of the second separate channel introducing the biological particles.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the junction 406 may have certain geometric features that at least partly determine the sizes of the droplets formed by the channel structure 400. The channel segment 402 can have a height, h₀ and width, w, at or near the junction 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the junction 406 can be inclined at an expansion angle, a. The expansion angle, a, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 408 entering the junction 406 can be between about 0.04 microliters (μL)/minute (min) and about 40 L/min. In some instances, the flow rate of the aqueous fluid 408 entering the junction 406 can be between about 0.01 microliters (L)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

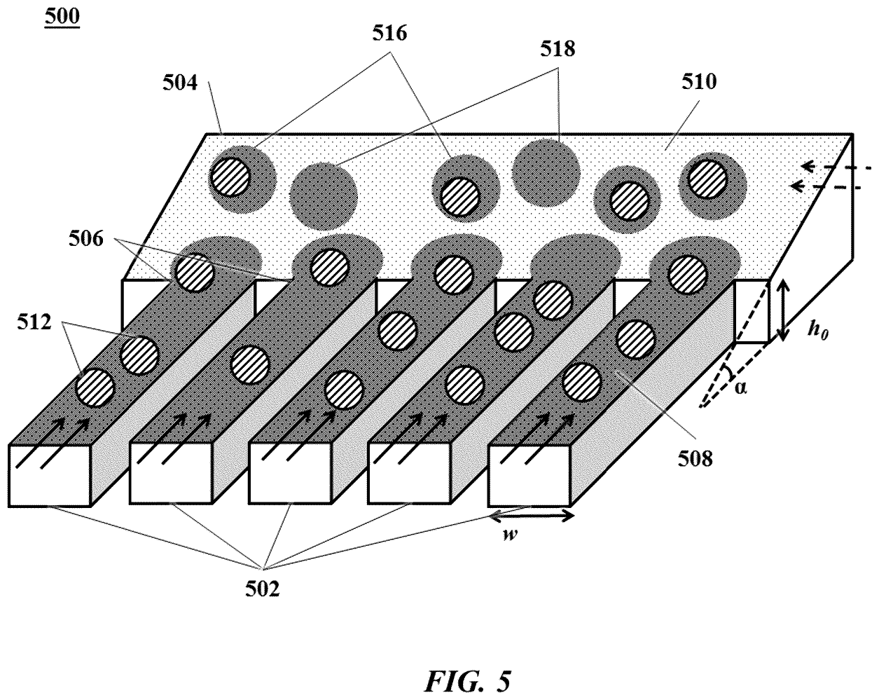

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctions. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the junction where the aqueous fluid 508 and the second fluid 510 meet, droplets can form based on factors such as the hydrodynamic forces at the junction, flow rates of the two fluids 508, 510, fluid properties, and certain geometric parameters (e.g., w, h₀, α, etc.) of the channel structure 500, as described elsewhere herein. A plurality of droplets can be collected in the reservoir 504 by continuously injecting the aqueous fluid 508 from the plurality of channel segments 502 through the plurality of junctions 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and α, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, when it is desirable to have a different distribution of droplet sizes, the geometric parameters for the plurality of channel segments 502 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

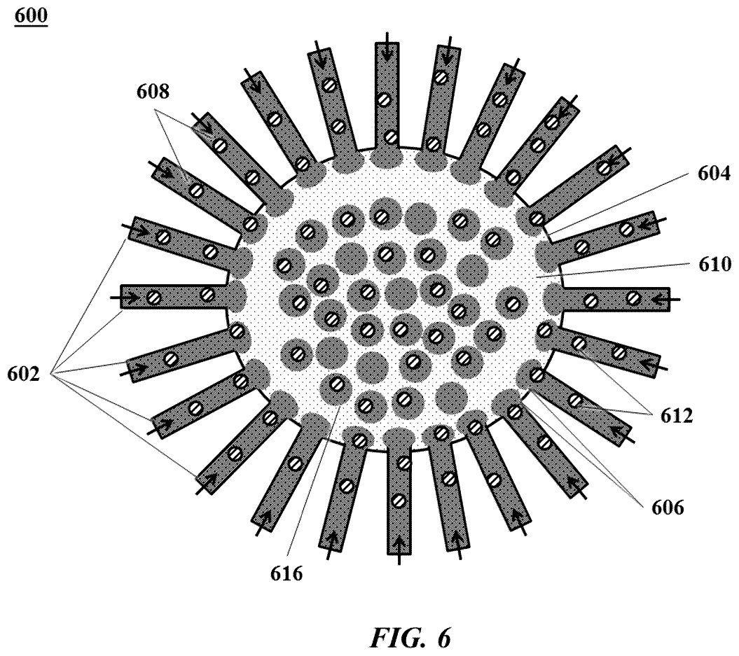

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 4 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctions. Alternatively, the second fluid 610 may be subjected and/or directed to flow in or out of the reservoir 604. For example, the reservoir 604 can be a channel directing the second fluid 610 from upstream to downstream, transporting the generated droplets.

In operation, the aqueous fluid 608 that includes suspended beads 612 may be transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the junction where the aqueous fluid 608 and the second fluid 610 meet, droplets can form based on factors such as the hydrodynamic forces at the junction, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctions 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, a (not shown in FIG. 6) at or near each channel junction. Each channel segment of the plurality of channel segments 602 may have a width, w, and a height, h₀, at or near the channel junction. The geometric parameters, w, h₀, and α, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, when it is desirable to have a different distribution of droplet sizes, the geometric parameters for the plurality of channel segments 602 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle injected into the droplets may or may not have uniform size.

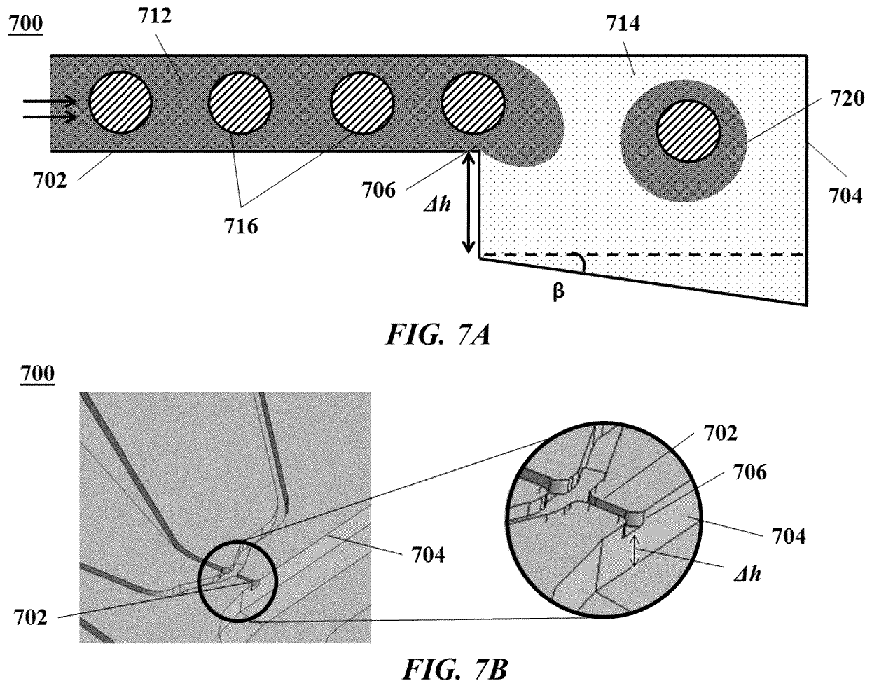

FIG. 7A shows a cross-section view of another example of a microfluidic channel structure with a geometric feature for controlled partitioning. A channel structure 700 can include a channel segment 702 communicating at a channel junction 706 (or intersection) with a reservoir 704. In some instances, the channel structure 700 and one or more of its components can correspond to the channel structure 100 and one or more of its components. FIG. 7B shows a perspective view of the channel structure 700 of FIG. 7A.

An aqueous fluid 712 comprising a plurality of particles 716 may be transported along the channel segment 702 into the junction 706 to meet a second fluid 714 (e.g., oil, etc.) that is immiscible with the aqueous fluid 712 in the reservoir 704 to create droplets 720 of the aqueous fluid 712 flowing into the reservoir 704. At the junction 706 where the aqueous fluid 712 and the second fluid 714 meet, droplets can form based on factors such as the hydrodynamic forces at the junction 706, relative flow rates of the two fluids 712, 714, fluid properties, and certain geometric parameters (e.g., 4h, etc.) of the channel structure 700. A plurality of droplets can be collected in the reservoir 704 by continuously injecting the aqueous fluid 712 from the channel segment 702 at the junction 706.

A discrete droplet generated may comprise one or more particles of the plurality of particles 716. As described elsewhere herein, a particle may be any particle, such as a bead, cell bead, gel bead, biological particle, macromolecular constituents of biological particle, or other particles. Alternatively, a discrete droplet generated may not include any particles.

In some instances, the aqueous fluid 712 can have a substantially uniform concentration or frequency of particles 716. As described elsewhere herein (e.g., with reference to FIG. 4), the particles 716 (e.g., beads) can be introduced into the channel segment 702 from a separate channel (not shown in FIG. 7). The frequency of particles 716 in the channel segment 702 may be controlled by controlling the frequency in which the particles 716 are introduced into the channel segment 702 and/or the relative flow rates of the fluids in the channel segment 702 and the separate channel. In some instances, the particles 716 can be introduced into the channel segment 702 from a plurality of different channels, and the frequency controlled accordingly. In some instances, different particles may be introduced via separate channels. For example, a first separate channel can introduce beads and a second separate channel can introduce biological particles into the channel segment 702. The first separate channel introducing the beads may be upstream or downstream of the second separate channel introducing the biological particles.

In some instances, the second fluid 714 may not be subjected to and/or directed to any flow in or out of the reservoir 704. For example, the second fluid 714 may be substantially stationary in the reservoir 704. In some instances, the second fluid 714 may be subjected to flow within the reservoir 704, but not in or out of the reservoir 704, such as via application of pressure to the reservoir 704 and/or as affected by the incoming flow of the aqueous fluid 712 at the junction 706. Alternatively, the second fluid 714 may be subjected and/or directed to flow in or out of the reservoir 704. For example, the reservoir 704 can be a channel directing the second fluid 714 from upstream to downstream, transporting the generated droplets.

The channel structure 700 at or near the junction 706 may have certain geometric features that at least partly determine the sizes and/or shapes of the droplets formed by the channel structure 700. The channel segment 702 can have a first cross-section height, h₁, and the reservoir 704 can have a second cross-section height, h₂. The first cross-section height, h₁, and the second cross-section height, h₂ may be different, such that at the junction 706, there is a height difference of Δh. The second cross-section height, h₂, may be greater than the first cross-section height, h₁. In some instances, the reservoir may thereafter gradually increase in cross-section height, for example, the more distant it is from the junction 706. In some instances, the cross-section height of the reservoir may increase in accordance with expansion angle, β, at or near the junction 706. The height difference, Δh, and/or expansion angle, β, can allow the tongue (portion of the aqueous fluid 712 leaving channel segment 702 at junction 706 and entering the reservoir 704 before droplet formation) to increase in depth and facilitate decrease in curvature of the intermediately formed droplet. For example, droplet size may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, β, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

In some instances, the flow rate of the aqueous fluid 712 entering the junction 706 can be between about 0.04 microliters (μL)/minute (min) and about 40 μL/min. In some instances, the flow rate of the aqueous fluid 712 entering the junction 706 can be between about 0.01 microliters (μL)/minute (min) and about 100 μL/min. Alternatively, the flow rate of the aqueous fluid 712 entering the junction 706 can be less than about 0.01 μL/min. Alternatively, the flow rate of the aqueous fluid 712 entering the junction 706 can be greater than about 40 μL/min, such as 45 μL/min, 50 μL/min, 55 μL/min, 60 μL/min, 65 L/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 712 entering the junction 706. The second fluid 714 may be stationary, or substantially stationary, in the reservoir 704. Alternatively, the second fluid 714 may be flowing, such as at the above flow rates described for the aqueous fluid 712.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

While FIGS. 7A and 7B illustrate the height difference, Δh, being abrupt at the junction 706 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the junction 706, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIGS. 7A and 7B illustrate the expanding reservoir cross-section height as linear (e.g., constant expansion angle, β), the cross-section height may expand non-linearly. For example, the reservoir may be defined at least partially by a dome-like (e.g., hemispherical) shape having variable expansion angles. The cross-section height may expand in any shape.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

### Computer Systems

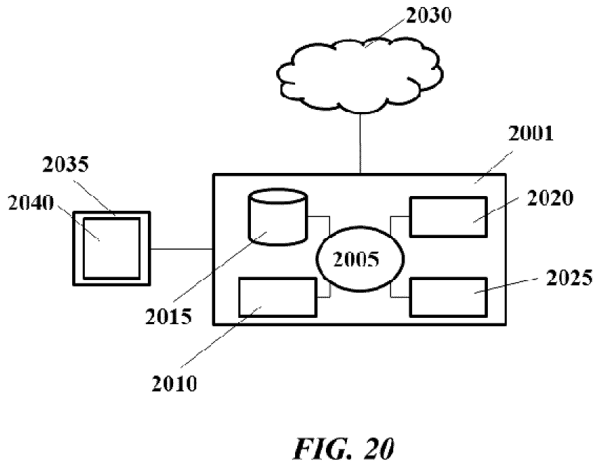

The present disclosure provides computer systems that are programmed to implement methods of the disclosure. FIG. 20 shows a computer system 2001 that is programmed or otherwise configured to control a microfluidics system (e.g., fluid flow), (ii) sort occupied droplets from unoccupied droplets, (iii) polymerize droplets, (iv) perform sequencing applications, and/or (v) generate and maintain sequencing libraries. The computer system 2001 can regulate various aspects of the present disclosure, such as, for example, regulating fluid flow rate in one or more channels in a microfluidic structure, regulating polymerization application units, etc. The computer system 2001 can be an electronic device of a user or a computer system that is remotely located with respect to the electronic device. The electronic device can be a mobile electronic device.

The computer system 2001 includes a central processing unit (CPU, also “processor” and “computer processor” herein) 2005, which can be a single core or multi core processor, or a plurality of processors for parallel processing. The computer system 2001 also includes memory or memory location 2010 (e.g., random-access memory, read-only memory, flash memory), electronic storage unit 2015 (e.g., hard disk), communication interface 2020 (e.g., network adapter) for communicating with one or more other systems, and peripheral devices 2025, such as cache, other memory, data storage and/or electronic display adapters. The memory 2010, storage unit 2015, interface 2020 and peripheral devices 2025 are in communication with the CPU 2005 through a communication bus (solid lines), such as a motherboard. The storage unit 2015 can be a data storage unit (or data repository) for storing data. The computer system 2001 can be operatively coupled to a computer network (“network”) 2030 with the aid of the communication interface 2020. The network 2030 can be the Internet, an internet and/or extranet, or an intranet and/or extranet that is in communication with the Internet. The network 2030 in some cases is a telecommunication and/or data network. The network 2030 can include one or more computer servers, which can enable distributed computing, such as cloud computing. The network 2030, in some cases with the aid of the computer system 2001, can implement a peer-to-peer network, which may enable devices coupled to the computer system 2001 to behave as a client or a server.

The CPU 2005 can execute a sequence of machine-readable instructions, which can be embodied in a program or software. The instructions may be stored in a memory location, such as the memory 2010. The instructions can be directed to the CPU 2005, which can subsequently program or otherwise configure the CPU 2005 to implement methods of the present disclosure. Examples of operations performed by the CPU 2005 can include fetch, decode, execute, and writeback.

The CPU 2005 can be part of a circuit, such as an integrated circuit. One or more other components of the system 2001 can be included in the circuit. In some cases, the circuit is an application specific integrated circuit (ASIC).

The storage unit 2015 can store files, such as drivers, libraries and saved programs. The storage unit 2015 can store user data, e.g., user preferences and user programs. The computer system 2001 in some cases can include one or more additional data storage units that are external to the computer system 2001, such as located on a remote server that is in communication with the computer system 2001 through an intranet or the Internet.

The computer system 2001 can communicate with one or more remote computer systems through the network 2030. For instance, the computer system 2001 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 2001 via the network 2030.

Methods as described herein can be implemented by way of machine (e.g., computer processor) executable code stored on an electronic storage location of the computer system 2001, such as, for example, on the memory 2010 or electronic storage unit 2015. The machine executable or machine readable code can be provided in the form of software. During use, the code can be executed by the processor 2005. In some cases, the code can be retrieved from the storage unit 2015 and stored on the memory 2010 for ready access by the processor 2005. In some situations, the electronic storage unit 2015 can be precluded, and machine-executable instructions are stored on memory 2010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 2001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2001 can include or be in communication with an electronic display 2035 that comprises a user interface (UI) 2040 for providing, for example, results of sequencing analysis, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 2005. The algorithm can, for example, e.g., perform a nucleic acid sequencing assay, etc.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-58. (canceled)

59. A method for nucleic acid processing, comprising:
(a) providing:
(i) a biological particle comprising (1) a vector comprising a guide ribonucleic acid (gRNA) sequence or (2) a gRNA molecule comprising said gRNA sequence;
(ii) a nucleic acid barcode molecule comprising an adapter sequence;
(iii) a first primer molecule comprising a sequence complementary to said adapter sequence; and
(iv) a second primer molecule;
(b) using (i) said first primer molecule and said vector or said gRNA molecule to generate a first product comprising a sequence complementary to said gRNA sequence and said sequence complementary to said adapter sequence, and (ii) said second primer molecule and said first product or a derivative thereof to generate a second product; and
(c) using said second product or a derivative thereof and said nucleic acid barcode molecule to generate a barcoded nucleic acid molecule.

60. The method of claim 59, wherein said biological particle is a cell, cell bead, or cell nucleus.

61. The method of claim 59, wherein said biological particle further comprises a deoxyribonucleic acid (DNA) molecule.

62. The method of claim 61, further comprising processing an open chromatin structure of said biological particle with a transposase to yield said DNA molecule.

63. The method of claim 62, wherein said processing occurs in a partition.

64. The method of claim 62, wherein said transposase is included in a transposase-nucleic acid complex that comprises (i) a first transposase nucleic acid molecule comprising a first transposon end sequence and a first sequencing primer or portion thereof, or a complement thereof and (ii) a second transposase nucleic acid molecule comprising a second transposon end sequence and a second sequencing primer or portion thereof, or a complement thereof.

65. The method of claim 64, wherein said first transposon end sequence and said second transposon end sequence are the same, and wherein said first transposon end sequence and said second transposon end sequence are hybridized to complementary sequences.

66. The method of claim 61, wherein (a) comprises providing a plurality of nucleic acid barcode molecules, including said nucleic acid barcode molecule, wherein said plurality of nucleic acid barcode molecules comprises an additional nucleic acid barcode molecule, wherein said nucleic acid barcode molecule and said additional nucleic acid barcode molecule comprise a common barcode sequence.

67. The method of claim 66, wherein (c) comprises using said DNA molecule and said additional nucleic acid barcode molecule to generate an additional barcoded nucleic acid molecule, wherein said barcoded nucleic acid molecule and said additional barcoded nucleic acid molecule comprise said common barcode sequence or a complement thereof.

68. The method of claim 59, further comprising, prior to (a), delivering to said biological particle a molecule comprising (i) a sequence of said vector or said gRNA molecule or (ii) a sequence complementary to a sequence of said vector or said gRNA molecule.

69. The method of claim 59, wherein said vector is a plasmid vector or viral vector.

70. The method of claim 59, wherein said vector or said gRNA molecule comprises a barcode sequence that identifies said gRNA sequence.

71. The method of claim 59, wherein said nucleic acid barcode molecules is coupled to a bead, and optionally wherein said bead is a gel bead.

72. The method of claim 71, wherein said nucleic acid barcode molecule is releasable from said bead upon application of a stimulus.

73. The method of claim 59, wherein generating said barcoded nucleic acid molecule comprises performing a ligation reaction, a primer extension reaction, a nucleic acid amplification reaction, or a combination thereof.

74. The method of claim 59, wherein said nucleic acid barcode molecule comprises a unique molecular identifier sequence, a sequencing primer or portion thereof, or a combination thereof.

75. The method of claim 59, further comprising, subsequent to (c), using (i) said barcoded nucleic acid molecule or derivative thereof to generate a plurality of amplification products.

76. The method of claim 59, wherein said first primer molecule or said second primer molecule is coupled to a particle, and optionally wherein (c) is performed within said partition

77. A computer system that is programed or otherwise configured to implement a method for nucleic acid processing,
(a) wherein the computer system is programed or otherwise configured to provide:
(i) a biological particle comprising (1) a vector comprising a guide ribonucleic acid (gRNA) sequence or (2) a gRNA molecule comprising said gRNA sequence;
(ii) a nucleic acid barcode molecule comprising an adapter sequence;
(iii) a first primer molecule comprising a sequence complementary to said adapter sequence; and
(iv) a second primer molecule;
(b) wherein the computer system is programed or otherwise configured to use (i) said first primer molecule and said vector or said gRNA molecule to generate a first product comprising a sequence complementary to said gRNA sequence and said sequence complementary to said adapter sequence, and (ii) said second primer molecule and said first product or a derivative thereof to generate a second product; and
(c) wherein the computer system is programed or otherwise configured to use said second product or a derivative thereof and said nucleic acid barcode molecule to generate a barcoded nucleic acid molecule.

78. The computer system of claim 77, wherein the computer system comprises a central processing unit (CPU).
